(19) United States
(12) Patent Application Publication
(10) Pub. No.: US 2020/0262919 A1
(43) Pub. Date: Aug. 20, 2020

(54) COMPOSITIONS AND METHODS FOR TREATING DIFFUSE LARGE B CELL LYMPHOMA

(71) Applicants: MERCK SHARP & DOHME CORP., Rahway, NJ (US); AMGEN INC., Thousand Oaks, CA (US)

(72) Inventors: Zachary ZIMMERMAN, Newbury Park, CA (US); Xiaohong Alicia ZHANG, Thousand Oaks, CA (US); Peter Christopher HOLLAND, N. Arlington, VA (US); Janet FRANKLIN, Woodland Hills, CA (US); Gregory FRIBERG, Westlake Village, CA (US)

(21) Appl. No.: 16/648,568

(22) PCT Filed: Oct. 12, 2018
(86) PCT No.: PCT/US18/55667

§ 371 Date: Mar. 18, 2020

Related U.S. Application Data
(60) Provisional application No. 62/571,870, filed on Oct. 13, 2017.

(51) Int. Cl. C07K 16/28; A61P 35/00

(52) U.S. Cl. CPC C07K 16/2809; A61K 2039/507; A61P 35/00; C07K 16/2818

(57) ABSTRACT

Methods and compositions for treating diffuse large B cell lymphoma (DLBCL) using a combination of blinatumomab and/or a blinatumomab variant and pembrolizumab, a pembrolizumab variant and/or an antigen-binding fragment thereof, are provided.

### RELATED APPLICATION

This application is a 35 U.S.C. § 371 filing of International Patent Application No. PCT/US2018/055667, filed Oct. 12, 2018, which claims the benefit of priority of U.S. Provisional Patent Application Ser. No. 62/571,870, filed Oct. 13, 2017, is the entire disclosures of which are hereby incorporated by reference.

### FIELD OF THE INVENTION

The present invention relates to the field of cancer therapeutics. In particular, the present invention relates to the treatment of relapsed or refractory diffuse large B cell lymphoma (DLBCL) using a combination therapy comprising blinatumomab and/or a blinatumomab variant, and pembrolizumab, a pembrolizumab variant and/or an antigen-binding fragment thereof.

### BACKGROUND

The annual incidence of non-Hodgkin lymphoma (NHL) in Europe and the USA is estimated to be 15 to 20 cases/100,000 (Fisher and Fisher, 2004). DLBCL is the most common lymphoid malignancy in adults, accounting for 31% of all NHL in Western countries and 37% of all B-cell tumors worldwide (NHL classification project, Blood 1997; Swerdlow et al, WHO classification 2016). The peak incidence of DLBCL is in the seventh decade (Martelli et al, 2013), with incidences increasing from 0.3/100.000/y (35-39 years) to 26.6/100,000/y (80-84 years; Morgan et al, 1997).

According to the World Health Organization (WHO) classification, DLBCL corresponds to a group of lymphoid malignancies composed of large cells with vesicular nuclei, prominent nucleoli, basophilic cytoplasm and an unusually high proliferation rate. Diffuse large B-cell lymphoma is biologically and clinically heterogeneous, with subgroups defined by morphology, immunophenotype, genetic alterations, and transcriptional patterns. Although most cases arise de novo, some are progression or transformation of less aggressive lymphoma, e.g., chronic lymphocytic leukemia or follicular lymphoma (Hartge and Wang, 2004). Despite this heterogeneity, and with the exception of the primary central nervous system (CNS) DLBCL, DLBCL is generally treated in a similar way (Gisselbrecht et al, 2010).

Overall, DLBCLs are aggressive but potentially curable malignancies. Cure rate is particularly high in patients with limited disease, with a 5-year progression free survival (PFS) ranging from 80 to 85%. Patients with advanced disease or symptomatic disease have a 5-year PFS of approximately 50%.

The choice of the first line treatment for patients with DLBCL is based on the individual IPI score and age. This leads to 3 major subgroups of DLBCL patients: elderly patients (>60 years, aaIPI=0-3), young patients with low risk (≤60 years, aaIPI=0-1) and young patients with high risk (≤60 years, aaIPI=2-3; Martelli et al, 2013). Rituximab cyclophosphamide, doxorubicin, vincristine, and prednisone (R-CHOP) given every 14 or 21 days is the cornerstone of first-line therapy for DLBCL (Zelenetz et al, 2016; Tilly et al, 2015), particularly for elderly patients and younger patients with low risk features. For elderly patients, the introduction of a “pre-phase” consisting of vincristine and prednisone may help reduce toxicities. Younger patients with low risk features may also be treated with rituximab, doxorubicin, cyclophosphamide, vincristine, bleomycin, and prednisone (RACVBP) without radiotherapy or R-CHOP21 with radiotherapy for bulky disease. Young patients with high risk represent the greatest current challenge in the front-line treatment of DLBCL. Around 30% of these patients are refractory to front-line R-CHOP. Several options in addition to R-CHOP are being considered, including enrollment in clinical trials or use of high dose chemotherapy with autologous hematopoietic stem cell transplantation (HSCT). Autologous HSCT is currently only recommended in eligible patients with DLBCL who did not achieve complete response (CR) after first line chemotherapy or in patients with chemosensitive relapse (Barosi et al, 2005).

Despite the improvements observed since the introduction of rituximab into front-line treatments, relapse is observed in 10-20% of patients with low IPI and 30-50% in high IPI patients. Various salvage regimens are currently used in r/r DLBCL. The CORAL study demonstrated no differences in response rates when using either rituximab, ifosfamide, carboplatin, etoposide (RICE) or rituximab, dexamethasone, cytarabine (also known as Ara-C) and cisplatin (R-DHAP) followed by autologous HSCT, with an overall response rate (ORR) of 63%. One third of patients did not respond to chemotherapy and only one half were able to proceed to autologous HSCT. Outcomes were particularly poor for patients that had received prior rituximab or had relapsed within 1 year of diagnosis (Gisselbrecht et al, 2010). Allogeneic HSCT is considered for a select group of patients with relapsed DLBCL (Friedberg, 2011). However, this treatment is associated with a high treatment related mortality rate (up to ˜25%).

For patients who have an inadequate response to, or who are not candidates for, intensive salvage regimens or HSCT, prognosis is poor with no defined standard of care. A clear need exists in the art for new methods and compositions for treating DLBCL.

### SUMMARY

The present disclosure is based on the discovery that combination therapy comprising blinatumomab and pembrolizumab, a pembrolizumab variant and/or an antigen-binding fragment thereof is useful in the treatment of diffuse large B cell lymphoma (DLBCL).

Accordingly, in one aspect, a method of treating DLBCL in a subject comprising administering blinatumomab or a blinatumomab variant to the subject, and administering pembrolizumab, a pembrolizumab variant or an antigen-binding fragment thereof to the subject, is provided.

In certain exemplary embodiments, the DLBCL is refractory to previous therapy or is relapsed after previous therapy.

In certain exemplary embodiments, the blinatumomab or the blinatumomab variant is administered to the subject systemically, e.g., by continuous intravenous infusion (CIVI). In other exemplary embodiments, the pembrolizumab, the pembrolizumab variant or the antigen-binding fragment thereof is administered to the subject systemically, e.g., by IV.

In certain exemplary embodiments, a first dose of the blinatumomab or the blinatumomab variant is administered to the subject prior to the administration of a first dose of the pembrolizumab, the pembrolizumab variant or the antigen-binding fragment thereof or concomitant with the administration of a first dose of the pembrolizumab, the pembrolizumab variant or the antigen-binding fragment thereof.

In certain exemplary embodiments, the blinatumomab or the blinatumomab variant is administered daily. In certain exemplary embodiments, a secondary dose of pembrolizumab, pembrolizumab variant or antigen-binding fragment thereof is administered approximately 21 days after the first dose of the pembrolizumab, pembrolizumab variant or antigen-binding fragment thereof. In certain exemplary embodiments, one or more additional secondary doses of pembrolizumab, pembrolizumab variant or antigen-binding fragment thereof are administered approximately every 21 days.

In certain exemplary embodiments, the pembrolizumab, the pembrolizumab variant or the antigen-binding fragment thereof is administered at a dose of about 200 mg. In certain exemplary embodiments, the blinatumomab or the blinatumomab variant is administered at an initial dose of at least about 9 μg. In certain exemplary embodiments, the blinatumomab or the blinatumomab variant is administered at a maintenance dose of about 28 μg, about 56 μg or about 112 μg.

In certain exemplary embodiments, the blinatumomab or the blinatumomab variant is administered in a first treatment cycle, followed by a treatment-free cycle, followed by one or more consolidation cycles.

In certain exemplary embodiments, the first treatment cycle is between about 49 and about 63 days. In certain exemplary embodiments, the first treatment cycle is about 56 days.

In certain exemplary embodiments, the treatment-free cycle is between about 14 and about 28 days. In certain exemplary embodiments, the treatment-free cycle is about 21 days.

In certain exemplary embodiments, the one or more consolidation cycles are each between about 14 and about 28 days. In certain exemplary embodiments, the one or more consolidation cycles are each about 21 days.

In certain exemplary embodiments, the first dose of the blinatumomab or the blinatumomab variant is administered to the subject on day 1 and the first dose of the pembrolizumab, the pembrolizumab variant or the antigen-binding fragment thereof is administered to the subject on day 1. In other exemplary embodiments, the first dose of the blinatumomab or the blinatumomab variant is administered to the subject on day 1 and the first dose of the pembrolizumab, the pembrolizumab variant or the antigen-binding fragment thereof is administered to the subject on about day 15. In still other exemplary embodiments, the first dose of the blinatumomab or the blinatumomab variant is administered to the subject on day 1 and the first dose of the pembrolizumab, the pembrolizumab variant or the antigen-binding fragment thereof is administered to the subject on about day 19.

In another aspect, a method of treating DLBCL in a subject comprising administering a dose of about 9 μg blinatumomab or a blinatumomab variant to the subject on each of treatment days 1 to 7, and administering an initial dose of about 200 mg pembrolizumab, a pembrolizumab variant or an antigen-binding fragment thereof to the subject on treatment day 1, and one or more subsequent doses of about 200 mg pembrolizumab, a pembrolizumab variant or an antigen-binding fragment thereof approximately every 21 days, is provided.

In certain exemplary embodiments, the method further comprises administering a dose of about 28 μg blinatumomab or a blinatumomab variant to the subject on each of treatment days 8 to 14, and optionally a dose of about 112 μg blinatumomab or a blinatumomab variant to the subject on each of treatment days 22 to 56, or a dose of about 56 μg blinatumomab or a blinatumomab variant to the subject on each of treatment days 15 to 56. In other exemplary embodiments, the method further comprises administering a dose of about 28 μg blinatumomab or a blinatumomab variant to the subject on each of treatment days 8 to 56.

In certain exemplary embodiments, the method further comprises a treatment-free cycle in which blinatumomab or a blinatumomab variant is not administered to the subject for between about 14 and about 28 days, optionally wherein the treatment-free cycle is about 21 days and/or further comprising one or more consolidated cycles wherein about 29 μg, about 56 μg or about 112 μg of blinatumomab or a blinatumomab variant is administered to the subject daily for between about 14 and about 28 days. In other exemplary embodiments, the one or more consolidated cycles are each about 21 days.

In another aspect, a method of treating DLBCL in a subject comprising administering a dose of about 9 μg blinatumomab or a blinatumomab variant to the subject on each of days 1 to 7 of a first treatment cycle, and administering an initial dose of about 200 mg pembrolizumab, a pembrolizumab variant or an antigen-binding fragment thereof to the subject on day 15 of the first treatment cycle, and one or more subsequent doses of about 200 mg pembrolizumab, a pembrolizumab variant or an antigen-binding fragment thereof approximately every 21 days, is provided.

In certain exemplary embodiments, the method further comprises administering a dose of about 28 μg blinatumomab or a blinatumomab variant to the subject on each of treatment days 8 to 56.

In certain exemplary embodiments, the method further comprises a treatment-free cycle in which blinatumomab or a blinatumomab variant is not administered to the subject for between about 14 and about 28 days, optionally wherein the treatment-free cycle is about 21 days and/or further comprising one or more consolidated cycles wherein about 29 μg, about 56 μg or about 112 μg of blinatumomab or a blinatumomab variant is administered to the subject daily for between about 14 and about 28 days. In other exemplary embodiments, the one or more consolidated cycles are each about 21 days.

In another aspect, a method of treating DLBCL in a subject comprising administering a dose of about 9 μg blinatumomab or a blinatumomab variant to the subject on each of days 1 to 7 of a first treatment cycle, and administering an initial dose of about 200 mg pembrolizumab, a pembrolizumab variant or an antigen-binding fragment thereof to the subject on day 19 of the first treatment cycle, and one or more subsequent doses of about 200 mg pembrolizumab, a pembrolizumab variant or an antigen-binding fragment thereof approximately every 21 days, is provided.

In certain exemplary embodiments, the method comprises administering a dose of about 28 μg blinatumomab or a blinatumomab variant to the subject on each of days 8 to 14 of the first treatment cycle, optionally administering a dose of about 112 μg blinatumomab or a blinatumomab variant to the subject on each of days 22 to 56 of the first treatment cycle or administering a dose of about 56 μg blinatumomab or a blinatumomab variant to the subject on each of days 15 to 56 of the first treatment cycle. In other exemplary embodiments, the method comprises administering a dose of about 28 μg blinatumomab or a blinatumomab variant to the subject on each of days 8 to 56 of the first treatment cycle.

In certain exemplary embodiments, the method further comprises a treatment-free cycle in which blinatumomab or a blinatumomab variant is not administered to the subject for between about 14 and about 28 days, optionally wherein the treatment-free cycle is about 21 days and/or further comprising one or more consolidated cycles wherein about 29 μg, about 56 μg or about 112 μg of blinatumomab or a blinatumomab variant is administered to the subject daily for between about 14 and about 28 days. In other exemplary embodiments, the one or more consolidated cycles are each about 21 days.

In another aspect, a method of treating DLBCL in a subject comprising administering a dose of about 9 μg blinatumomab or a blinatumomab variant to the subject on each of days 1 to 7 of a first treatment cycle, and a dose of about 28 μg blinatumomab or a blinatumomab variant to the subject on each of days 8 to 56 of the first treatment cycle, and administering an initial dose of about 200 mg pembrolizumab, a pembrolizumab variant or an antigen-binding fragment thereof to the subject on treatment day 1, and one or more subsequent doses of about 200 mg pembrolizumab, a pembrolizumab variant or an antigen-binding fragment thereof approximately every 21 days, is provided.

In another aspect, a method of treating DLBCL in a subject comprising, administering a dose of about 9 μg blinatumomab or a blinatumomab variant to the subject on each of days 1 to 7 of a first treatment cycle, a dose of about 28 μg blinatumomab or a blinatumomab variant to the subject on each of days 8 to 14 of the first treatment cycle, and a dose of about 112 μg blinatumomab or a blinatumomab variant to the subject on each of days 15 to 56 of the first treatment cycle, and administering an initial dose of about 200 mg pembrolizumab, a pembrolizumab variant or an antigen-binding fragment thereof to the subject on day 1 of the first treatment cycle, and one or more subsequent doses of about 200 mg pembrolizumab, a pembrolizumab variant or an antigen-binding fragment thereof approximately every 21 days, is provided.

In another aspect, a method of treating DLBCL in a subject comprising administering a dose of about 9 μg blinatumomab or a blinatumomab variant to the subject on each of days 1 to 7 of a first treatment cycle, a dose of about 28 μg blinatumomab or a blinatumomab variant to the subject on each of days 8 to 14 of the first treatment cycle, and a dose of about 56 μg blinatumomab or a blinatumomab variant to the subject on each of days 15 to 56 of the first treatment cycle, and administering an initial dose of about 200 mg pembrolizumab, a pembrolizumab variant or an antigen-binding fragment thereof to the subject on day 1 of the first treatment cycle, and one or more subsequent doses of about 200 mg pembrolizumab, a pembrolizumab variant or an antigen-binding fragment thereof approximately every 21 days, is provided.

In another aspect, a method of treating DLBCL in a subject comprising administering a dose of about 9 μg blinatumomab or a blinatumomab variant to the subject on each of days 1 to 7 of a first treatment cycle, and a dose of about 28 μg blinatumomab or a blinatumomab variant to the subject on each of days 8 to 56 of the first treatment cycle, and administering an initial dose of about 200 mg pembrolizumab, a pembrolizumab variant or an antigen-binding fragment thereof to the subject on day 15 of the first treatment cycle, and one or more subsequent doses of about 200 mg pembrolizumab, a pembrolizumab variant or an antigen-binding fragment thereof approximately every 21 days, is provided.

In another aspect, a method of treating DLBCL in a subject comprising administering a dose of about 9 μg blinatumomab or a blinatumomab variant to the subject on each of days 1 to 7 of the first treatment cycle, a dose of about 28 μg blinatumomab or a blinatumomab variant to the subject on each of days 8 to 14 of the first treatment cycle, and a dose of about 112 μg blinatumomab or a blinatumomab variant to the subject on each of days 15 to 56 of the first treatment cycle, and administering an initial dose of about 200 mg pembrolizumab, a pembrolizumab variant or an antigen-binding fragment thereof to the subject on day 19 of the first treatment cycle, and one or more subsequent doses of about 200 mg pembrolizumab, a pembrolizumab variant or an antigen-binding fragment thereof approximately every 21 days, is provided.

In another aspect, a method of treating DLBCL in a subject comprising administering a dose of about 9 μg blinatumomab or a blinatumomab variant to the subject on each of days 1 to 7 of the first treatment cycle, a dose of about 28 μg blinatumomab or a blinatumomab variant to the subject on each of days 8 to 14 of the first treatment cycle, and a dose of about 56 jpg blinatumomab or a blinatumomab variant to the subject on each of days 15 to 56 of the first treatment cycle, and administering an initial dose of about 200 mg pembrolizumab, a pembrolizumab variant or an antigen-binding fragment thereof to the subject on day 19 of the first treatment cycle, and one or more subsequent doses of about 200 mg pembrolizumab, a pembrolizumab variant or an antigen-binding fragment thereof approximately every 21 days, is provided.

In another aspect, a method of treating DLBCL in a subject comprising administering a dose of about 28 μg, about 56 μg, or about 112 μg blinatumomab or a blinatumomab variant to the subject daily starting at treatment day 1, and administering an initial dose of about 200 mg pembrolizumab, a pembrolizumab variant or an antigen-binding fragment thereof approximately every 21 days starting at treatment day 1, is provided.

In another aspect, a method of treating DLBCL in a subject comprising administering a dose of about 28 μg, about 56 μg, or about 112 μg blinatumomab or a blinatumomab variant to the subject daily starting at treatment day 1, and administering an initial dose of about 200 mg pembrolizumab, a pembrolizumab variant or an antigen-binding fragment thereof approximately every 21 days starting at treatment day 15, is provided.

In another aspect, a method of treating DLBCL in a subject comprising administering a dose of about 28 μg, about 56 μg, or about 112 μg blinatumomab or a blinatumomab variant to the subject daily starting at treatment day 1, and administering an initial dose of about 200 mg pembrolizumab, a pembrolizumab variant or an antigen-binding fragment thereof approximately every 21 days starting at treatment day 19, is provided.

In another aspect, blinatumomab or a blinatumomab variant for use in treating DLBCL in a subject in combination with pembrolizumab, a pembrolizumab variant or an antigen-binding fragment thereof, is provided.

In another aspect, pembrolizumab, a pembrolizumab variant or an antigen-binding fragment thereof for use in treating DLBCL in a subject in combination with blinatumomab or a blinatumomab variant is provided.

In certain exemplary embodiments, the DLBCL is refractory to previous therapy or is relapsed after previous therapy.

In certain exemplary embodiments, the blinatumomab or the blinatumomab variant is administered to the subject systemically, e.g., by continuous intravenous infusion (CIVI). In other exemplary embodiments, the pembrolizumab, the pembrolizumab variant or the antigen-binding fragment thereof is administered to the subject systemically, e.g., by IV.

In certain exemplary embodiments, a first dose of the blinatumomab or the blinatumomab variant is administered to the subject prior to the administration of a first dose of the pembrolizumab, the pembrolizumab variant or the antigen-binding fragment thereof or concomitant with the administration of a first dose of the pembrolizumab, the pembrolizumab variant or the antigen-binding fragment thereof.

In certain exemplary embodiments, the blinatumomab or the blinatumomab variant is administered daily. In certain exemplary embodiments, a secondary dose of pembrolizumab, pembrolizumab variant or antigen-binding fragment thereof is administered approximately 21 days after the first dose of the pembrolizumab, pembrolizumab variant or antigen-binding fragment thereof. In certain exemplary embodiments, one or more additional secondary doses of pembrolizumab, pembrolizumab variant or antigen-binding fragment thereof are administered approximately every 21 days.

In certain exemplary embodiments, the pembrolizumab, the pembrolizumab variant or the antigen-binding fragment thereof is administered at a dose of about 200 mg. In certain exemplary embodiments, the blinatumomab or the blinatumomab variant is administered at an initial dose of at least about 9 μg. In certain exemplary embodiments, the blinatumomab or the blinatumomab variant is administered at a maintenance dose of about 28 μg, about 56 μg or about 112 μg.

In certain exemplary embodiments, the blinatumomab or the blinatumomab variant is administered in a first treatment cycle, followed by a treatment-free cycle, followed by one or more consolidation cycles.

In certain exemplary embodiments, the first treatment cycle is between about 49 and about 63 days. In certain exemplary embodiments, the first treatment cycle is about 56 days.

In certain exemplary embodiments, the treatment-free cycle is between about 14 and about 28 days. In certain exemplary embodiments, the treatment-free cycle is about 21 days.

In certain exemplary embodiments, the one or more consolidation cycles are each between about 14 and about 28 days. In certain exemplary embodiments, the one or more consolidation cycles are each about 21 days.

In certain exemplary embodiments, the first dose of the blinatumomab or the blinatumomab variant is administered to the subject on day 1 and the first dose of the pembrolizumab, the pembrolizumab variant or the antigen-binding fragment thereof is administered to the subject on day 1. In other exemplary embodiments, the first dose of the blinatumomab or the blinatumomab variant is administered to the subject on day 1 and the first dose of the pembrolizumab, the pembrolizumab variant or the antigen-binding fragment thereof is administered to the subject on about day 15. In still other exemplary embodiments, the first dose of the blinatumomab or the blinatumomab variant is administered to the subject on day 1 and the first dose of the pembrolizumab, the pembrolizumab variant or the antigen-binding fragment thereof is administered to the subject on about day 19.

In another aspect, a medicament comprising blinatumomab or a blinatumomab variant for use in treating DLBCL in a subject in combination with pembrolizumab, a pembrolizumab variant or an antigen-binding fragment thereof, is provided.

In another aspect, a medicament comprising pembrolizumab, a pembrolizumab variant or an antigen-binding fragment thereof for use in treating DLBCL in a subject in combination with blinatumomab or a blinatumomab variant is provided.

In certain exemplary embodiments, the DLBCL is refractory to previous therapy or is relapsed after previous therapy.

In certain exemplary embodiments, the blinatumomab or the blinatumomab variant is administered to the subject systemically, e.g., by continuous intravenous infusion (CIVI). In other exemplary embodiments, the pembrolizumab, the pembrolizumab variant or the antigen-binding fragment thereof is administered to the subject systemically, e.g., by IV.

In certain exemplary embodiments, a first dose of the blinatumomab or the blinatumomab variant is administered to the subject prior to the administration of a first dose of the pembrolizumab, the pembrolizumab variant or the antigen-binding fragment thereof or concomitant with the administration of a first dose of the pembrolizumab, the pembrolizumab variant or the antigen-binding fragment thereof.

In certain exemplary embodiments, the blinatumomab or the blinatumomab variant is administered daily. In certain exemplary embodiments, a secondary dose of pembrolizumab, pembrolizumab variant or antigen-binding fragment thereof is administered approximately 21 days after the first dose of the pembrolizumab, pembrolizumab variant or antigen-binding fragment thereof. In certain exemplary embodiments, one or more additional secondary doses of pembrolizumab, pembrolizumab variant or antigen-binding fragment thereof are administered approximately every 21 days.

In certain exemplary embodiments, the pembrolizumab, the pembrolizumab variant or the antigen-binding fragment thereof is administered at a dose of about 200 mg. In certain exemplary embodiments, the blinatumomab or the blinatumomab variant is administered at an initial dose of at least about 9 μg. In certain exemplary embodiments, the blinatumomab or the blinatumomab variant is administered at a maintenance dose of about 28 μg, about 56 μg or about 112 μg.

In certain exemplary embodiments, the blinatumomab or the blinatumomab variant is administered in a first treatment cycle, followed by a treatment-free cycle, followed by one or more consolidation cycles.

In certain exemplary embodiments, the first treatment cycle is between about 49 and about 63 days. In certain exemplary embodiments, the first treatment cycle is about 56 days.

In certain exemplary embodiments, the treatment-free cycle is between about 14 and about 28 days. In certain exemplary embodiments, the treatment-free cycle is about 21 days.

In certain exemplary embodiments, the one or more consolidation cycles are each between about 14 and about 28 days. In certain exemplary embodiments, the one or more consolidation cycles are each about 21 days.

In certain exemplary embodiments, the first dose of the blinatumomab or the blinatumomab variant is administered to the subject on day 1 and the first dose of the pembrolizumab, the pembrolizumab variant or the antigen-binding fragment thereof is administered to the subject on day 1. In other exemplary embodiments, the first dose of the blinatumomab or the blinatumomab variant is administered to the subject on day 1 and the first dose of the pembrolizumab, the pembrolizumab variant or the antigen-binding fragment thereof is administered to the subject on about day 15. In still other exemplary embodiments, the first dose of the blinatumomab or the blinatumomab variant is administered to the subject on day 1 and the first dose of the pembrolizumab, the pembrolizumab variant or the antigen-binding fragment thereof is administered to the subject on about day 19.

The summary of the disclosure described above is non-limiting and other features and advantages of the disclosed biomarkers and methods will be apparent from the following drawings, the detailed description of the disclosure, the example and the claims.

### BRIEF DESCRIPTION OF THE DRAWINGS

FIG. 1 depicts the study design and treatment schema for blinatumomab and pembrolizumab combination therapy cohorts. DLT=dose limiting toxicity; MTD=maximum tolerated dose. The first cycle of blinatumomab will be 8 weeks in duration, followed by a 28-day (+3 days) blinatumomab treatment-free interval. A second consolidation cycle of blinatumomab will be 28 days in duration at the same dose as the first cycle, starting at 9 μg/day with weekly dose escalations until the target dose is reached, if subject has stable disease or partial/complete response after cycle 1. Pembrolizumab will be started on study day 15 for cohort Ia, will be started study day 1 for cohorts Ib, IIb, and IIIb, and will be started study day 19 for cohorts Ha and IIIa, and administered Q3 weeks until disease progression for up to 35 cycles. Part 1: To determine maximum tolerated dose (MTD) of blinatumomab in combination with pembrolizumab. The MTD will be defined as the dose level at which ≤1 of 6 subjects experience a dose limiting toxicity (DLT) or the maximum administered dose (MAD). ^{b}Part 2: Expansion cohort to estimate the efficacy of the combination of blinatumomab and pembrolizumab. Dosing will be determined based on the MTD of blinatumomab established in part 1. DLTs will be continuously monitored to ensure they do not reach a pre-defined threshold. ^{C}For cohorts Ia, Ha and IIIa, the DLT observation period will begin on the same day as the first dose of pembrolizumab (day 15 for Ia and day 19 for IIa and IIIa) and will continue for 42 days. For cohort Ib, the DLT observation period will begin on day 1 of the start of the combination of pembrolizumab/blinatumomab, and continue for 42 days. For cohorts IIb, and IIIb, the DLT observation period will begin once the blinatumomab target dose (28 μg/day on day 8, 112 μg/day on day 15, or 56 μg/day on day 15 for cohorts Ib, IIb, and IIIb, respectively) is reached and will continue for 28 days. A dose level review team (DLRT) will review the available data to determine if blinatumomab is safe and tolerable as defined by DLT criteria. ^{d}Dosing for the Part 2 expansion cohort will be based on the safety of the combination of blinatumomab and pembrolizumab and the MTD of blinatumomab in Part 1.

FIGS. 2A-2B schematically depict (FIG. 2A) blinatumomab structure and (FIG. 2B) the mode of action of blinatumomab.

FIG. 3 depicts a table showing the schedule of Assessments for Cohort Ia (and for Part 2 if MTD is Reached in Cohort Ia). AE=adverse event; CBC=complete blood count; CNS=central nervous system; CR=complete response; CSF=cerebrospinal fluid; CT=computer tomography; DLBCL=diffuse large B cell lymphoma; ECOG=Eastern Cooperative Oncology Group; FDG=fluorodeoxyglucose; FU=follow up; IV=intravenous; LTFU=long term follow-up; MRD=minimal residual disease; MRI=magnetic resonance imaging; MTD=maximum tolerated dose; NGS=next generation sequencing; PET=positron emission tomography; PK=pharmacokinetics; PRO=patient reported outcomes; SAE=serious adverse event. aA safety follow-up will occur 30 days (+7 days) after last dose of each protocol specified therapy. ^{b}All procedures completed on the first day of study treatment must be completed prior to the initiation of protocol-required therapy. ^{c}The initial dose of blinatumomab will be 9 μg/day and the dose will be escalated at weekly intervals until the target dose is reached. See FIG. 1. dPembrolizumab will be administered starting on study day 15 (21-day cycles).

FIG. 4 depicts a table showing the schedule of pembrolizumab dosing and related assessments for Cohort Ia (and for Part 2 if MTD is reached in Cohort Ia). CBC=completed blood count; FU=follow-up; MTD=maximum tolerated dose; PK=pharmacokinetic. Pembrolizumab anti-drug antibodies (serum) will be collected at pre-dose (trough) within 24 hours before the following infusions of pembrolizumab: 1 (study day 15), 2 (study day 36), 4 (study day 78), 6 (study day 120), 8 (study day 162), and every 4 infusions thereafter, and 30 days after discontinuation of pembrolizumab (or until the subject starts new anticancer therapy). ^{b}Pembrolizumab PK pre-dose samples (serum) will be collected within 24 hours before the following infusions of pembrolizumab: on the first day of pembrolizumab treatment (study day 15) and at pembrolizumab cycles 2 (study day 36), 4 (study day 78), 6 (study day 120), and 8 (study day 162), then every 4 cycles. (See FIG. 3.) CPK post-dose samples will be collected 30 minutes post infusion on the first day of pembrolizumab treatment (study day 15), then on days 2 (study day 16), 8 (study day 22), and 15 (study day 29) of cycle 1 of pembrolizumab, cycle 8 day 1 (study day 162), and 30 days after discontinuation of pembrolizumab. (See FIG. 3.)

FIG. 5 depicts a table showing the schedule of pembrolizumab dosing and related assessments for cohorts Ib, IIb, and IIIb (and for part 2 if MTD is reached in any of these cohorts). CBC=completed blood count; FU=follow-up; MTD=maximum tolerated dose; PK=pharmacokinetic. Pembrolizumab anti-drug antibodies (serum) will be collected at pre-dose (trough) within 24 hours before the following infusions of pembrolizumab: 1 (study day 1), 2 (study day 22), 4 (study day 64), 6 (study day 106), 8 (study day 148), and every 4 infusions thereafter, and 30 days after discontinuation of pembrolizumab (or until the subject starts new anticancer therapy). Pembrolizumab PK pre-dose samples (serum) will be collected within 24 hours before the following infusions of pembrolizumab: on the first day of pembrolizumab (study day 1) and at pembrolizumab cycles 2 (study day 22), 4 (study day 64), 6 (study day 106), and 8 (study day 148), then every 4 cycles. (See FIG. 5.) PK post-dose samples will be collected 30 minutes post infusion on the first day of pembrolizumab (study day 1), then on days 2 (study day 2), 8 (study day 8), and 15 (study day 15) of pembrolizumab cycle 1, cycle 8 day 1 (study day 148), and 30 days after discontinuation of pembrolizumab. (See FIG. 5.)

FIG. 6 depicts a table showing the schedule of pembrolizumab dosing and related to assessments for Cohorts Ha and IIIa (and for Part 2 if MTD is reached in either of these Cohorts). CBC=completed blood count; FU=follow-up; MTD=maximum tolerated dose; PK=pharmacokinetic. ^{a}Pembrolizumab anti-drug antibodies (serum) will be collected at pre-dose (trough) within 24 hours before the following infusions of pembrolizumab: 1 (study day 19), 2 (study day 40), 4 (study day 82), 6 (study day 124), 8 (study day 166), and every 4 infusions thereafter, and 30 days after discontinuation of pembrolizumab (or until the subject starts new anticancer therapy). ^{b}Pembrolizumab PK pre-dose samples (serum) will be collected within 24 hours before the following infusions of pembrolizumab: on the first day of pembrolizumab treatment (study day 19) and at pembrolizumab cycles 2 (study day 40), 4 (study day 82), 6 (study day 124), and 8 (study day 166), then every 4 cycles. (See FIG. 7.) CPK post-dose samples will be collected 30 minutes post infusion on the first day of pembrolizumab treatment (study day 19), then on days 2 (study day 20), 8 (study day 26), and 15 (study day 33) of cycle 1 of pembrolizumab, cycle 8 day 1 (study day 166), and 30 days after discontinuation of pembrolizumab. (See FIG. 7.)

FIG. 7 depicts a table showing the revised Cheson Criteria for evaluation of extramedullary disease.

FIG. 8 depicts a table showing response assessment using the Lugano Classification. A 5-point scale is used (Deauville):

1, no uptake above background;

2, uptake ≤mediastinum;

3, uptake >mediastinum but ≤liver;

4, uptake moderately>liver;

5, uptake markedly higher than liver and/or new lesions;

X, new areas of uptake unlikely to be related to lymphoma.

FIG. 9 depicts a status overview of cohort 1a.

FIG. 10 depicts an overview of a cohort 1a subject.

### DETAILED DESCRIPTION OF CERTAIN EXEMPLARY EMBODIMENTS

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About” when used to modify a numerically defined parameter (e.g., the dosage of blinatumomab, a blinatumomab variant, pembrolizumab, a pembrolizumab variant and/or an antigen-binding fragment thereof, or the length of treatment time with blinatumomab, a blinatumomab variant, pembrolizumab, pembrolizumab variant and/or an antigen-binding fragment thereof) means that the parameter may vary by 1%, 2%, 3%, 4%, 5%, 6%, 7%, 8%, 9% or 10% above or below the stated numerical value for that parameter.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

As used herein, the term “antibody” refers to any form of antibody that exhibits the desired biological or binding activity. Thus, it is used in the broadest sense and specifically covers, but is not limited to, monoclonal antibodies (including full-length monoclonal antibodies), polyclonal antibodies, multi-specific antibodies (e.g., bispecific antibodies), humanized antibodies, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)).

The variable regions of each light/heavy chain pair form the antibody binding site. Thus, in general, an intact antibody has two binding sites. Except in bifunctional or bispecific antibodies, the two binding sites are, in general, the same.

“Variable regions” or “V region” as used herein means the segment of IgG chains which is variable in sequence between different antibodies. It extends to Kabat residue 109 in the light chain and 113 in the heavy chain.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia et al., (1987) J Mol. Biol. 196:901-917 or Chothia et al., (1989) Nature 342:878-883.

As used herein, the term “hypervariable region” refers to the amino acid residues of an antibody that are responsible for antigen-binding. The hypervariable region comprises amino acid residues from a CDR (i.e. LCDR1, LCDR2 and LCDR3 in the light chain variable domain and HCDR1, HCDR2 and HCDR3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). As used herein, the term “framework” or “FR” residues refers to those variable domain residues other than the hypervariable region residues defined herein as CDR residues.

As used herein, unless otherwise indicated, “antibody fragment” or “antigen-binding fragment” refers to antigen-binding fragments of antibodies, i.e., antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multi-specific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, useful in the present invention will bind to the target protein with an affinity that is at least two fold greater, preferably at least ten times greater, more preferably at least 20 times greater, and most preferably at least 100 times greater than the affinity with non-target proteins. As used herein, an antibody is said to bind specifically to a polypeptide comprising a given amino acid sequence, e.g. the amino acid sequence of a mature human PD-1 or human PD-L1 molecule, mature human CD19 or mature human CD3, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Biotherapeutic agent” means a biological molecule, such as an antibody and/or an sc-Fv, that blocks ligand/receptor signaling in any biological pathway that supports tumor maintenance and/or growth or suppresses the anti-tumor immune response.

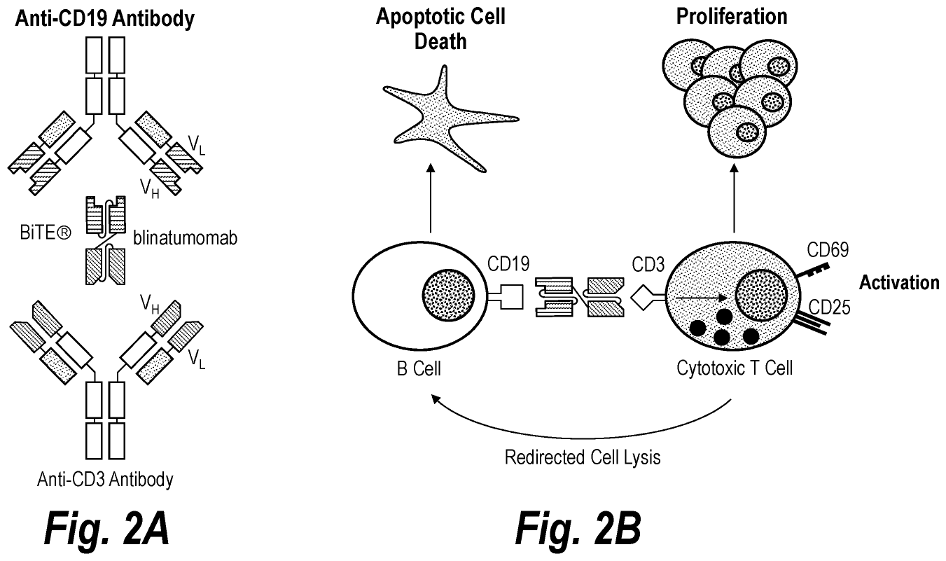

The term “blinatumomab,” as used herein, refers to a CD19×CD3 bispecific antibody construct also known as a BiTE®, or bispecific T-cell engagers (Dreier T, Lorenczewski G, Brandl C, et al. Extremely potent, rapid and co-stimulation independent cytotoxic T-cell response against lymphoma cells catalyzed by a single chain bispecific antibody. Int J Cancer. 2002; 100(6):690-697; Schlereth B, Kleindienst P, Fichtner I, et al. Potent inhibition of local and disseminated tumor growth in immunocompetent mouse models by a bispecific antibody construct specific for Murine CD3. Cancer Immunol Immunother. 2006; 55(7):785-796). Blinatumomab is a BiTE® antibody construct with dual binding specificities (FIG. 2). T cells are bound by its anti CD3 moiety, whereas B lymphoblasts and other B cells are bound by the anti-CD19 moiety. This unique feature of blinatumomab allows it to transiently connect malignant cells with T-cells, thereby inducing T cell mediated killing of the bound malignant cell.

Blinatumomab specifically targets cells that express CD19, a marker solely expressed by B cells, including B-precursor acute lymphoblastic leukemia (ALL) cells, with an affinity of 1.6×10⁻⁹ M. Blinatumomab recruits and activates T cells via a lower affinity interaction with CD3 (8.7×10¹ M). These activated T cells then induce a half-maximal target cell lysis ranging in vitro between 10 to 100 μg/mL showing blinatumomab to be an extremely potent molecule (Dreier et al, 2002).

During the course of tumor cell elimination, activated T cells synthesize and secrete pro-inflammatory cytokines as tumor necrosis factor-alpha (TNF-α), interferon-gamma (IFN-γ), interleukin (IL)-6, and IL-2, which might induce symptoms such as fever or decreases of blood pressure. In vitro data demonstrate cytokine release as a result of blinatumomab-mediated T cell activation, which can be attenuated by corticosteroids without impairing the cytotoxic activity. In vivo data indicate cytokine release to be most prominent following the first dose of blinatumomab.

Due to its unique ability to redirect T cells via CD3 towards a CD19+ tumor cell lysis, blinatumomab can elicit repeated target cell elimination by cytotoxic T cells and a polyclonal response of previously primed CD4+ and C8+ T cells. The anti-tumor activity is effective within a wide range of effector to target (E:T) ratios.

In the absence of CD19+ target cells, neither cytotoxicity nor release of cytokines will occur. Blinatumomab acts strictly in a target cell specific and dependent manner, with regard to cytotoxic action. The presence of both CD19⁺ target cells and T cells are required for its cytotoxic activity.

As of July 2017, blinatumomab (BLINCYTO®) is indicated for the treatment of relapsed or refractory B cell precursor ALL in the United States. It is indicated in multiple countries outside of the United States for Philadelphia chromosome negative relapsed or refractory B-cell precursor ALL (e.g., European Union, Mexico, Canada, Norway, Iceland, Australia, and South Korea).

As used herein, a “CD19×CD3 bispecific antibody construct” (including a CD19×CD3 bispecific single chain antibody—sometimes both terms are used interchangeably herein) denotes a single polypeptide chain comprising two binding domains. Such CD19×CD3 bispecific single chain antibody constructs are preferred in the context of the methods/dosage regimen of the present invention. Each binding domain comprises at least one variable region from an antibody heavy chain (“VH or H region”), wherein the VH region of the first binding domain specifically binds to the CD3 epsilon molecule, and the VH region of the second binding domain specifically binds to CD19. The two binding domains are optionally linked to one another by a short polypeptide spacer. A non-limiting example for a polypeptide spacer is Gly-Gly-Gly-Gly-Ser (G-G-G-G-S) (SEQ ID NO: 33) and repeats thereof. Each binding domain may additionally comprise one variable region from an antibody light chain (“VL or L region”), the VH region and VL region within each of the first and second binding domains being linked to one another via a polypeptide linker, for example of the type disclosed and claimed in EP 623679 B1, but in any case long enough to allow the VH region and VL region of the first binding domain and the VH region and VL region of the second binding domain to pair with one another such that, together, they are able to specifically bind to the respective first and second binding domains. Such CD19×CD3 bispecific single chain antibody constructs are described in great detail in WO 99/54440 and WO 2004/106381 and WO2008/119565.

The term “binding domain” characterizes in connection with the present invention a domain of a polypeptide which specifically binds to/interacts with a given target structure/antigen/epitope. Thus, the binding domain is an “antigen-interaction-site.” The term “antigen-interaction-site” defines, in accordance with the present invention, a motif of a polypeptide, which is able to specifically interact with a specific antigen or a specific group of antigens, e.g., the identical antigen in different species. Said binding/interaction is also understood to define a “specific recognition.” The term “specifically recognizing” means in accordance with this invention that the antibody molecule is capable of specifically interacting with and/or binding to at least two, preferably at least three, more preferably at least four amino acids of an antigen, e.g., the human CD3 antigen, the human CD19 antigen, and/or the human PD-1 antigen, as defined herein. Such binding may be exemplified by the specificity of a “lock-and-key-principle.” Thus, specific motifs in the amino acid sequence of the binding domain and the antigen bind to each other as a result of their primary, secondary or tertiary structure as well as the result of secondary modifications of said structure. The specific interaction of the antigen-interaction-site with its specific antigen may result as well in a simple binding of said site to the antigen. Moreover, the specific interaction of the binding domain/antigen-interaction-site with its specific antigen may alternatively result in the initiation of a signal, e.g., due to the induction of a change of the conformation of the antigen, an oligomerization of the antigen, etc. A preferred example of a binding domain in line with the present invention is an antibody. The binding domain may be a monoclonal or polyclonal antibody or derived from a monoclonal or polyclonal antibody.

The human CD19 protein has the UniProt Accession No. P15391. The human CD3 protein comprises gamma, delta, epsilon and zeta subunits that have UniProt Accession Nos. P09693 (CD3G), P04234 (CD3D), P07766 (CD3E) and P20963 (CD3Z).

In certain exemplary embodiments, the bispecific antibody construct applied in the methods/dosage regimens of the present invention has the domain arrangement VL(CD19)-VH(CD 19)-VH(CD3)-VL(CD3).

It is, however, also envisaged that the methods of the invention can be carried out with CD19×CD3 bispecific single chain antibody constructs of other domain arrangements, such as

VH(CD19)-VL(CD19)-VH(CD3)-VL(CD3),

VL(CD19)-VH(CD19)-VL(CD3)-VH(CD3),

VH(CD19)-VL(CD19)-VL(CD3)-VH(CD3),

VL(CD3)-VH(CD3)-VH(CD19)-VL(CD19),

VH(CD3)-VL(CD3)-VH(CD19)-VL(CD19),

VL(CD3)-VH(CD3)-VL(CD19)-VH(CD19), or

VH(CD3)-VL(CD3)-VL(CD19)-VH(CD19).

TABLE 1
CD3 and CD19 heavy chain
and light chain CDR sequences.

| CDR | Sequence | SEQ ID NO |
|---|---|---|
| CD3 CDR-H1 | GYTFTRYTMH | 1 |
| CD3 CDR-H2 | YINPSRGYTNYNQKFKD | 2 |
| CD3 CDR-H3 | YYDDHYCLDY | 3 |
| CD3 CDR-L1 | RASSSVSYMN | 4 |
| CD3 CDR-L2 | DTSKVAS | 5 |
| CD3 CDR-L3 | QQWSSNPLT | 6 |
| CD19 CDR-H1 | GYAFSSYWMN | 7 |
| CD19 CDR-H2 | QIWPGDGDTNYNGKFKG | 8 |
| CD19 CDR-H3 | RETTTVGRYYYAMDY | 9 |
| CD19 CDR-L1 | KASQSVDYDGDSYLN | 10 |
| CD19 CDR-L2 | DASNLVS | 11 |
| CD19 CDR-L3 | QQSTEDPWT | 12 |

In certain exemplary embodiments, a CD19×CD3 bispecific antibody construct applied in the methods of the present invention comprises:

(a) the anti-CD3 CDRs of a heavy chain comprising CD3 CDR-H1 set forth as GYTFTRYTMH (SEQ ID NO: 1), CD3 CDR-H2 set forth as YINPSRGYTNYNQKFKD (SEQ ID NO: 2), and CD3 CDR-H3 set forth as YYDDHYCLDY (SEQ ID NO: 3); and/or

(b) the anti-CD3 CDRs of a light chain comprising CD3 CDR-L1 set forth as RASSSVSYMN (SEQ ID NO: 4), CD3 CDR-L2 set forth as DTSKVAS (SEQ ID NO: 5), and CD3 CDR-L3 set forth as QQWSSNPLT (SEQ ID NO: 6); and/or

(c) the anti-CD19 CDRs of a heavy chain comprising CD19 CDR-H1 set forth as GYAFSSYWMN (SEQ ID NO: 7), CD19 CDR-H2 set forth as QIWPGDGDTNYNGKFKG (SEQ ID NO: 8), and CD19 CDR-H3 set forth as RETTVGRYYYAMDY (SEQ ID NO: 9); and/or

(d) the anti-CD19 CDRs of a light chain comprising CD19 CDR-L1 set forth as KASQSVDYDGDSYLN (SEQ ID NO: 10), CD19 CDR-L2 set forth as DASNLVS (SEQ ID NO: 11), and CD19 CDR-L3 set forth as QQSTEDPWT (SEQ ID NO: 12).

In certain exemplary embodiments, the CD19×CD3 bispecific single chain antibody construct applied in the methods of the present invention comprises the CD3 CDRs of the heavy and light chain. In other exemplary embodiments, the CD19×CD3 bispecific antibody construct applied in the methods of the present invention comprises the CD3 CDRs of the heavy and light chain as well as the CD19 CDRs of the heavy and light chain.

Alternatively, it is preferred that the CD19×CD3 bispecific single chain antibody construct applied in the methods of the present invention comprises:

(a) a CD19 variable heavy chain set forth as
(SEQ ID NO: 13)
QVQLQQSGAELVRPGSSVKISCKASGYAFSSYWMNWVKQRPGQGLEWIGQ
IWPGDGDTNYNGKFKGKATLTADESSSTAYMQLSSLASEDSAVYFCARRE
TTTVGRYYYAMDYWGQGTTVTVSS
(encoded by the nucleotide sequence set forth as
caggtgcagc tgcagcagtc tggggctgag ctggtgaggc
ctgggtcctc agtgaagatt tcctgcaagg cttctggcta
tgcattcagt agctactgga tgaactgggt gaagcagagg
cctggacagg gtcttgagtg gattggacag atttggcctg
gagatggtga tactaactac aatggaaagt tcaagggtaa
agccactctg actgcagacg aatcctccag cacagcctac
atgcaactca gcagcctagc atctgaggac tctgcggtct
atttctgtgc aagacgggag actacgacgg taggccgtta
ttactatgct atggactact ggggccaagg gaccacggtc
accgtctcct cc (SEQ ID NO: 14));
and/or
(b) a CD19 variable light chain set forth as
(SEQ ID NO: 15)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIK
(encoded by the nucleotide sequence set forth as
gatatccagc tgacccagtc tccagcttct ttggctgtgt
ctctagggca gagggccacc atctcctgca aggccagcca
aagtgttgat tatgatggtg atagttattt gaactggtac
caacagattc caggacagcc acccaaactc ctcatctatg
atgcatccaa tctagtttct gggatcccac ccaggtttag
tggcagtggg tctgggacag acttcaccct caacatccat
cctgtggaga aggtggatgc tgcaacctat cactgtcagc
aaagtactga ggatccgtgg acgttcggtg gagggaccaa
gctcgagatc aaa (SEQ ID NO: 16));
and/or
(c) a CD3 variable heavy chain set forth as
(SEQ ID NO: 17)
DIKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSS
(encoded by the nucleotide sequence set forth as
gatatcaaac tgcagcagtc aggggctgaa ctggcaagac
ctggggcctc agtgaagatg tcctgcaaga cttctggcta
cacctttact aggtacacga tgcactgggt aaaacagagg
cctggacagg gtctggaatg gattggatac attaatccta
gccgtggtta tactaattac aatcagaagt tcaaggacaa
ggccacattg actacagaca aatcctccag cacagcctac
atgcaactga gcagcctgac atctgaggac tctgcagtct
attactgtgc aagatattat gatgatcatt actgccttga
ctactggggc caaggcacca ctctcacagt ctcctca (SEQ ID
NO: 18));
and/or
(d) a CD3 variable light chain set forth as
(SEQ ID NO: 19)
DIQLTQSPAIMSASPGEKVTMTCRASSSVSYMNWYQQKSGTSPKRWIYDT
SKVASGVPYRFSGSGSGTSYSLTISSMEAEDAATYYCQQWSSNPLTFGAG
TKLELK
(encoded by the nucleotide sequence set forth as
gacattcagc tgacccagtc tccagcaatc atgtctgcat
ctccagggga gaaggtcacc atgacctgca gagccagttc
aagtgtaagt tacatgaact ggtaccagca gaagtcaggc
acctccccca aaagatggat ttatgacaca tccaaagtgg
cttctggagt cccttatcgc ttcagtggca gtgggtctgg
gacctcatac tctctcacaa tcagcagcat ggaggctgaa
gatgctgcca cttattactg ccaacagtgg agtagtaacc
cgctcacgtt cggtgctggg accaagctgg agctgaaa (SEQ ID
NO: 20)).

In certain exemplary embodiments, the CD19×CD3 bispecific single chain antibody construct applied in the methods of the present invention comprises the CD19 variable heavy and light chain and/or the CD3 variable heavy and light chain. In certain exemplary embodiments, the CD19×CD3 bispecific single chain antibody construct applied in the methods of the present invention comprises the CD19 variable heavy and light chain as well as the CD3 variable heavy and light chain.

In certain exemplary embodiments, said bispecific single chain antibody construct comprises an amino acid sequence selected from the group consisting of

(a) an amino acid sequence set forth as
(SEQ ID NO: 21)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVSS
GGGGSDIKLQQSGAELARPGASVKMSCKTSGYTFTRYTMHWVKQRPGQGL
EWIGYINPSRGYTNYNQKFKDKATLTTDKSSSTAYMQLSSLTSEDSAVYY
CARYYDDHYCLDYWGQGTTLTVSSVEGGSGGSGGSGGSGGVDDIQLTQSP
AIMSASPGEKVTMTCRASSSVSYMNWYQQKSGTSPKRWIYDTSKVASGVP
YRFSGSGSGTSYSLTISSMEAEDAATYYCQQWSSNPLTFGAGTKLELK;
(b) an amino acid sequence encoded by a nucleic
acid sequence set forth as
(SEQ ID NO: 22)
gatatccagc tgacccagtc tccagcttct ttggctgtgt
ctctagggca gagggccacc atctcctgca aggccagcca
aagtgttgat tatgatggtg atagttattt gaactggtac
caacagattc caggacagcc acccaaactc ctcatctatg
atgcatccaa tctagtttct gggatcccac ccaggtttag
tggcagtggg tctgggacag acttcaccct caacatccat
cctgtggaga aggtggatgc tgcaacctat cactgtcagc
aaagtactga ggatccgtgg acgttcggtg gagggaccaa
gctcgagatc aaaggtggtg gtggttctgg cggcggcggc
tccggtggtg gtggttctca ggtgcagctg cagcagtctg
gggctgagct ggtgaggcct gggtcctcag tgaagatttc
ctgcaaggct tctggctatg cattcagtag ctactggatg
aactgggtga agcagaggcc tggacagggt cttgagtgga
ttggacagat ttggcctgga gatggtgata ctaactacaa
tggaaagttc aagggtaaag ccactctgac tgcagacgaa
tcctccagca cagcctacat gcaactcagc agcctagcat
ctgaggactc tgcggtctat ttctgtgcaa gacgggagac
tacgacggta ggccgttatt actatgctat ggactactgg
ggccaaggga ccacggtcac cgtctcctcc ggaggtggtg
gatccgatat caaactgcag cagtcagggg ctgaactggc
aagacctggg gcctcagtga agatgtcctg caagacttct
ggctacacct ttactaggta cacgatgcac tgggtaaaac
agaggcctgg acagggtctg gaatggattg gatacattaa
tcctagccgt ggttatacta attacaatca gaagttcaag
gacaaggcca cattgactac agacaaatcc tccagcacag
cctacatgca actgagcagc ctgacatctg aggactctgc
agtctattac tgtgcaagat attatgatga tcattactgc
cttgactact ggggccaagg caccactctc acagtctcct
cagtcgaagg tggaagtgga ggttctggtg gaagtggagg
ttcaggtgga gtcgacgaca ttcagctgac ccagtctcca
gcaatcatgt ctgcatctcc aggggagaag gtcaccatga
cctgcagagc cagttcaagt gtaagttaca tgaactggta
ccagcagaag tcaggcacct cccccaaaag atggatttat
gacacatcca aagtggcttc tggagtccct tatcgcttca
gtggcagtgg gtctgggacc tcatactctc tcacaatcag
cagcatggag gctgaagatg ctgccactta ttactgccaa
cagtggagta gtaacccgct cacgttcggt gctgggacca
agctggagct gaaa;

(c) an amino acid sequence encoded by a nucleic acid sequence having at least 70%, 80%, 90%, 95% or 99% identity to a nucleic acid sequence of (b), wherein said amino acid sequence is capable of specifically binding to CD3 and CD19; and

(d) an amino acid sequence encoded by a nucleic acid sequence which is degenerate as a result of the genetic code to a nucleotide sequence of(b), wherein said amino acid sequence is capable of specifically binding to CD3 and CD19.

The terms “cancer,” “cancerous,” or “malignant” refer to or describe the physiological condition in mammals that is typically characterized by unregulated cell growth.

In certain exemplary embodiments, a cancer is a lymphoma. As used herein, a “lymphoma” refers to a group of blood cell cancers that develop from lymphocytes. Lymphomas include, but are not limited to, Hodgkin lymphoma, and non-Hodgkin lymphoma, e.g., B cell lymphoma (e.g., diffuse large B cell lymphoma (DLBCL), follicular lymphoma, chronic lymphocytic leukemia (CLL), small lymphocytic lymphoma (SLL), mantle cell lymphoma (MCL), marginal zone lymphomas, Burkitt lymphoma, lymphoplasmacytic lymphoma, hairy cell leukemia, primary central nervous system lymphoma and the like), T cell lymphoma (e.g., precursor T-lymphoblastic lymphoma/leukemia, peripheral T-cell lymphomas and the like) or NK cell lymphoma. An example of a lymphoma according to certain exemplary embodiments that is responsive to blinatumomab/pembrolizumab combination therapy is DLBCL.

In certain exemplary embodiments, the tumorous mass of lymph node tissue and/or extranodal lymphoma caused by DLBCL is characterized by tumors having a size of more than about 10×10 mm, more than about 15×15 mm, or more than about 20×20 mm, or even larger. Likewise, if the tumor is determined in three dimensions, the tumorous mass of lymph node tissue and/or extranodal lymphoma caused by DLBCL is can be characterized by tumors having a size of more than about 10×10×10 mm, more than about 15×15×15 mm, more than about 20×20×20 mm, or even larger.

Lymph node tissue preferably includes lymph nodes (including lymph node regions and/or lymph structures) and spleen. Lymph node regions can be defined as an area of lymph nodes and the surrounding tissue. Examples include the cervical nodes in the neck, the axillary nodes in the armpit, the inguinal nodes in the groin, and/or the mediastinal nodes in the chest. Lymph structures can be defined as organs or structures that are part of the lymphatic system, such as the lymph nodes, spleen, and thymus gland.

Accordingly, in some of the foregoing embodiments, the patient has, inter alia, at least one, two, three, four, five or more enlarged lymph node(s).

As used herein, and “extranodal lymphoma” refers to a lymphoma in which, after routine staging procedures, there is either no or only “minor” nodal involvement along with a clinically “dominant” extranodal component, to which primary treatment must often be directed. In certain exemplary embodiments, extranodal lymphoma includes the central nervous system (CNS), cutaneous tissue, breast, lungs, liver, gastrointestinal tract, genitourinary tract, ocular tissue, bone marrow and/or bones.

“CDR” or “CDRs” as used herein means complementarity determining region(s) in an immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, luteinizing hormone-releasing hormone agonists, anti-androgens, aromatase inhibitors, EGFR inhibitors, VEGF inhibitors, anti-sense oligonucleotides that that inhibit expression of genes implicated in abnormal cell proliferation or tumor growth. Chemotherapeutic agents useful in the treatment methods of the present invention include cytostatic and/or cytotoxic agents.

“Chothia” as used herein means an antibody numbering system described in Al-Lazikani et al., JMB 273:927-948 (1997), incorporated by reference herein.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering (or substantially altering) the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity.

“Comprising” or variations such as “comprise,” “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, if a gene signature score is defined as the composite RNA expression score for a set of genes that consists of a specified list of genes, the skilled artisan will understand that this gene signature score could include the RNA expression level determined for one or more additional genes, preferably no more than three additional genes, if such inclusion does not materially affect the predictive power.

“Framework region” or “FR” as used herein means the immunoglobulin variable regions excluding the CDR regions.

“Homology” refers to sequence similarity between two polypeptide sequences when they are optimally aligned. When a position in both of the two compared sequences is occupied by the same amino acid monomer subunit, e.g., if a position in a light chain CDR of two different antibodies is occupied by alanine, then the two antibodies are homologous at that position. The percent of homology is the number of homologous positions shared by the two sequences divided by the total number of positions compared ×100. For example, if 8 of 10 of the positions in two sequences are matched or homologous when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab,” as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Interferon gamma” and “IFNγ” (also called immune or type II interferon), refers to a pleiotropic cytokine involved in the regulation of nearly all phases of immune and inflammatory responses, including the activation, growth and differentiation of T-cells, B-cells, macrophages, NK cells and other cell types such as endothelial cells and fibroblasts. IFNγ enhances MHC expression on antigen-presenting cells, and also plays an important role in activating lymphocytes to enhance anti-tumor effects.

IFNγ can contribute to the containment of tumor progression and growth by increasing tumor antigen presentation to tumor-specific T cells and increasing susceptibility to NK cytotoxicity. In addition to promoting an immune response to the tumor, IFN-γ can also induce expression of tumor suppressing factors.

“Oligonucleotide” refers to a nucleic acid that is usually between 5 and 100 contiguous bases in length, and most frequently between 10-50, 10-40, 10-30, 10-25, 10-20, 15-50, 15-40, 15-30, 15-25, 15-20, 20-50, 20-40, 20-30 or 20-25 contiguous bases in length.

“Patient” or “subject” refers to any single subject for which therapy is desired or that is participating in a clinical trial, epidemiological study or used as a control, including humans, non-human primates, mammalian veterinary patients such as cattle, horses, dogs, cats and the like, and research animals such as non-human primates, rats, mice, dogs, rabbits and the like.

As used herein, “pembrolizumab” refers to a humanized monoclonal antibody that binds to and blocks PD-1. Pembrolizumab works by increasing the ability of the body's immune system to help detect and fight tumor cells by blocking the interaction between PD-1 and its ligands, PD-L1 and PD-L2, thereby activating T lymphocytes which may affect both tumor cells and healthy cells.

The sequence of human PD-1 has a UniProt Accession number of Q9UMF3.

Pembrolizumab monotherapy is known to treat melanoma, non-small cell lung cancer and squamous cell carcinoma of the head and neck in affected individuals having higher densities of baseline CD8+ T-cell infiltrations, IFNγ gene signature and PD-L1 expression than levels found in non-responsive individuals.

As used herein, “pembrolizumab” refers to a commercially available monoclonal antibody under the proprietary name of KEYTRUDA@ (Merck Sharp & Dohme Corp., Whitehouse Station, N.J.), described in WO2016196173 and U.S. Pat. Nos. 8,354,509 and 8,900,587, incorporated herein by reference in their entireties for all purposes, as well as variants and antigen-binding fragments thereof. Pembrolizumab can be characterized by one or any combination of the heavy chain domain, light chain domain, heavy chain variable domain, light chain variable domain, heavy chain complementarity-determining and light chain complementarity-determining sequences described Infra.

Pembrolizumab can comprise a heavy chain sequence set forth as QVQLVQSGVEVKKPGASVKVSCKASGYTFTNYYMYWVRQAPGQGLEWMGGINPS NGGTNFNEKFKNRVTLTIDSSTITAYMELKSLQFDDTAVYYCARRDYRFDMGFDY WGQGTIVTVSSASTKGPSVFPLAPCSRSTSESTAALGCLVKDYFPEPVTVSWNSGAL TSGVHTFPAVLQSSGLYSLSSVVTVPSSSLGTKTYTCNVDHKPSNTKVDKRVESKYG PPCPPCPAPEFLGGPSVFLFPPKPKDTLMISRTPEVTCVVVDVSQEDPEVQFNWYVDG VEVHNAKTKPREEQFNSTYRVVSVLTVLHQDWLNGKEYKCKVSNKGLPSSIEKTISK AKGQPREPQVYTLPPSQEEMTKNQVSLTCLVKGFYPSDIAVEWESNGQPENNYKTITP PVLDSDGSFFLYSRLTVDKSRWQEGNVFSCSVMHEALHNHYTQKSLSLSLGK (SEQ ID NO: 23), and a light chain sequence set forth as EIVLTQSPATLSLSPGERATLSCRASKGVSTSGYSYLHWYQQKPGQ APRLLIYLASYLESGVPARFSGSGSGTDFTLTISSLEPEDFAVYYCQHSRDLPLTFGGG TKVEIKRTVAAPSVFIFPPSDEQLKSGTASVVCLLNNFYPREAKVQWKVDNALQSGN SQESVTEQDSKDSTYSLSSTLTLSKADYEKHKVYACEVTHQGLSSPVTKSFNRGEC (SEQ ID NO: 24).

Pembrolizumab can comprise a heavy chain variable (VH) domain sequence set forth as QVQLVQSGVEVKKPGASVKVSCKASGYTFTNYYMYWVRQAPGQGLEWMGGINPS NGGTNFNEKFKNRVTLTDSSTITAYMELKSLQFDDTAVYYCARRDYRFDMGFDY WGQGTTVTVSS (SEQ ID NO: 25), and a light chain variable (VL) domain set forth as EIVLTQSPATLSLSPGERATLSCRASKGVSTSGYSYLHWYQQKPGQAPRLLIYLASYL ESGVPARFSGSGSGTDFTLTISSLEPEDFAVYYCQHSRDLPLTFGGGTKVEIK (SEQ ID NO: 26).

Pembrolizumab can comprise the following heavy chain complementarity-determining regions (HCDRs): NYYMY (HCDR1, SEQ ID NO: 27); GINPSNGGTNFN (HCDR2, SEQ ID NO: 28); and RDYRFDMGFDY (HCDR3, SEQ ID NO: 29).

Pembrolizumab can comprise the following light chain complementarity-determining regions (LCDRs): RASKGVSTSGYSYLH (LCDR1, SEQ ID NO: 30); LASYLES (LCDR2, SEQ ID NO: 31); and QHSRDLPLT (LCDR3, SEQ ID NO: 32).

In certain embodiments, pembrolizumab, a pembrolizumab variant or an antigen-binding fragment thereof is provided comprising heavy chain CDRs SEQ ID NOs: 27, 28 and 29 and light chain CDRs of SEQ ID NOs: 30, 31 and 32.

In other embodiments, pembrolizumab, a pembrolizumab variant or an antigen-binding fragment thereof is provided comprising heavy chain and light chain CDR sequences from a VH/VL sequence pair of SEQ ID NO: 25 and SEQ ID NO: 26.

In still other preferred embodiments, pembrolizumab, a pembrolizumab variant or an antigen-binding fragment thereof is provided comprising a heavy chain variable region comprising SEQ ID NO: 25 or a variant thereof and/or a light chain variable region comprising SEQ ID NO: 26 or a variant thereof. In other embodiments, the pembrolizumab variant or antigen-binding fragment thereof comprises a heavy chain variable region comprising as sequence with at least 80% sequence homology or identity (e.g., 80%, 85%, 90%, 95%, 98% or 99%) to SEQ ID NO: 25 and/or a light chain variable region comprising a sequence with at least 80% sequence homology or identify (e.g., 80%, 85%, 90%, 95%, 98% or 99%) to SEQ ID NO: 26.

As used herein, a “variant of a heavy chain variable region sequence” is a sequence that is identical to the reference sequence, except having up to 17 conservative amino acid substitutions in the framework region (i.e., outside of the CDRs), and preferably having fewer than ten, nine, eight, seven, six or five conservative amino acid substitutions in the framework region. As used herein, a “variant of a light chain variable region sequence” is a sequence that is identical to the reference sequence, except having up to five conservative amino acid substitutions in the framework region (i.e., outside of the CDRs), and preferably having fewer than four, three or two conservative amino acid substitution in the framework region.

In still other embodiments, pembrolizumab, a pembrolizumab variant or an antigen-binding fragment thereof is provided comprising a heavy chain comprising SEQ ID NO: 23 or a variant thereof and/or a light chain comprising SEQ ID NO: 24 or a variant thereof. In other embodiments, the pembrolizumab variant or antigen-binding fragment thereof comprises a heavy chain comprising as sequence with at least 80% sequence homology or identity (e.g., 80%, 85%, 90%, 95%, 98% or 99%) to SEQ ID NO: 23 and/or a light chain comprising a sequence with at least 80% sequence homology or identify (e.g., 80%, 85%, 90%, 95%, 98% or 99%) to SEQ ID NO: 24.

As used herein, a “blinatumomab variant” or a “pembrolizumab variant” refers to a monoclonal antibody which comprises heavy chain and light chain sequences that are identical to those of blinatumomab or pembrolizumab, respectively, except for having up to five conservative amino acid substitutions in the framework region (i.e., outside of the CDRs), and preferably has less than four, three or two conservative amino acid substitution in the framework region, and having up to 17 conservative amino acid substitutions in the framework region (i.e., outside of the CDRs), and preferably has less than ten, nine, eight, seven, six or five conservative amino acid substitutions in the framework region, and preferably has less than four, three or two conservative amino acid substitution in the framework region. In other words, blinatumomab and a blinatumomab variant, or pembrolizumab and a pembrolizumab variant, comprise identical CDR sequences, but differ from each other due to having a conservative amino acid substitution at no more than three or six other positions in their full-length light and heavy chain sequences, respectively. A blinatumomab variant is substantially the same as or better than blinatumomab with respect to the following properties: binding affinity to CD19, binding affinity to CD3 and neutralizing effect in vivo. A pembrolizumab variant is substantially the same as or better than pembrolizumab with respect to the following properties: binding affinity to PD-1 and neutralizing effect in vivo.

In certain embodiments, biosimilars of pembrolizumab are provided.

As used herein, the term “biosimilar” is used in a manner that is consistent with a working definition promulgated by the U.S. Food and Drug Administration (FDA), European Medicines Agency (EMA) and/or Health Canada, which define a biosimilar product to be one that is “highly similar” to a reference product (despite minor differences in clinically inactive components), or similar definition used by another regulatory agency worldwide. In practice, there should be no clinically meaningful differences between the reference product and the biosimilar product in terms of safety, purity, and potency. In certain embodiments, a double-blind, single-dose comparative pharmacokinetic (PK) crossover study is performed to compare pembrolizumab with a candidate biosimilar antibody to determine comparable bioavailability.

As used herein, the term “reference product,” is used to refer to commercially available pembrolizumab or commercially available blinatumomab.

“RECIST 1.1 Response Criteria” as used herein means the definitions set forth in Eisenhauer et al., E. A. et al., Eur. J Cancer 45:228-247 (2009) for target lesions or non-target lesions, as appropriate, based on the context in which response is being measured.

“Responder patient” when referring to a specific anti-tumor response to treatment with a combination therapy described herein, means the patient exhibited the anti-tumor response.

“Sample” when referring to a tumor or any other biological material referenced herein, means a sample that has been removed from the subject. Biological samples include body fluids (such as blood, serum, plasma, urine, saliva, synovial fluid, spinal fluid and the like) and tissue sources that have malignant CD19 positive lymphocytes. Methods for obtaining tissue biopsies and body fluids from patients are well known in the art. Generally, a biological sample which includes peripheral blood mononuclear cells (PBMCs), in particular B cells and T cells, is preferred as a source.

A sample which includes peripheral blood mononuclear cells (PBMCs), in particular B cells and T cells, is preferably taken from peripheral blood of a human patient. Other preferred samples are whole blood, serum, plasma or synovial fluid, with plasma or serum being most preferred.

Another preferred sample obtained from a patient is a lymph node biopsy. A lymph node biopsy is, for example, obtained with an excisional biopsy of an abnormal lymph node or a generous incisional biopsy of an involved organ. In some cases, cutting-needle biopsies can provide adequate tissue for diagnosis. In addition, an adequate bone marrow biopsy may be performed. Diagnosis can be supplemented by gene-expression profiling. More preferably, the diagnosis is preferably made by a hematopathologist with experience in diagnosing lymphomas, in particular DLBCL by, preferably applying the WHO classification of lymphoid neoplasma (see Table 1 on page 30 of the publication of Armitage in Blood (2007), Vol. 110 (1):29-36). It is sometimes also preferred to perform immunohistochemistry and on occasion to apply cytogenetics or fluorescent in situ hybridization (FISH) in order to clarify an initial diagnosis.

In one embodiment of the present invention DLBCL is diagnosed in accordance with the symptoms described herein and/or by applying the means and methods described herein such as lymph node biopsy, immunohistochemistry, cytogenetics, gene-profiling and/or FISH.

Once the diagnosis is made and, preferably confirmed, additional tests such as restaging by re-biopsy by a further experienced hematopathologist and/or further imaging studies including computer tomography, ultra sound imaging, and/or PET scan of the chest, abdomen and/or pelvis, are performed to obtain more information about the extent to which the disease has spread in the body. This process is called staging. The results of these tests will help determine the most effective course of treatment.

A number of staging tests are available to help determine which areas of the body have been affected by follicular lymphoma. Tests that may be done include: CT scan, blood tests, bone marrow biopsy and/or PET scan.

Staging involves dividing patients into groups (stages) based upon how much of the lymphatic system is involved at the time of diagnosis. Staging helps determine a person's prognosis and treatment options.

Stages of lymphoma can be defined as follows:

Stage I—Only one lymph node region is involved, or only one lymph structure is involved.

Stage II—Two or more lymph node regions or lymph node structures on the same side of the diaphragm are involved.

Stage III—Lymph node regions or structures on both sides of the diaphragm are involved.

Stage IV—There is widespread involvement of a number of organs or tissues other than lymph node regions or structures, such as the liver, lung, or bone marrow.

When a stage is assigned, it also includes a letter, A or B, to denote whether fever, weight loss, or night sweats are present. “A” means these symptoms are not present; “B” means they are. For example, a person with stage 1B disease has evidence of cancer in one lymph node region and has “B” symptoms (fever, weight loss and/or night sweats).

In the present invention, DLBCL is preferably staged in accordance with the criteria set out in Cheson et al. (2007), J. Clin. Oncol. 25(5):579-586.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Standard of care systemic anti-cancer therapy” refers to medically-accepted diagnostic and treatment processes that a clinician follows for a particular cancer in a particular patient that may include one or more biological therapies (e.g., immunotherapies) and/or one or more cytotoxic chemotherapies that would be readily known to one of skill in the art. As used herein, standard of care systemic anti-cancer therapy excludes blinatumomab/pembrolizumab combination therapy.

“Tissue Section” refers to a single part or piece of a tissue sample, e.g., a thin slice of tissue cut from a sample of a normal tissue or of a tumor.

“Treat” or “treating” DLBCL, as used herein, means to administer blinatumomab, a blinatumomab variant, pembrolizumab, a pembrolizumab variant or an antigen-binding fragment thereof, to a subject diagnosed with DLBCL to achieve at least one positive therapeutic effect, such as for example, reduced number of cancer cells, reduced tumor size, reduced rate of cancer cell infiltration into peripheral organs, or reduced rate of tumor metastasis or tumor growth.

Positive therapeutic effects in cancer can be measured in a number of ways (See, W. A. Weber, J. Null. Med. 50:1S-10S (2009); Eisenhauer et al., supra). In some preferred embodiments, response to blinatumomab, a blinatumomab variant, pembrolizumab, a pembrolizumab variant and/or an antigen-binding fragment thereof, is assessed using RECIST 1.1 criteria. In some embodiments, the treatment achieved by a therapeutically effective amount is any of a partial response (PR), a complete response (CR), progression free survival (PFS), disease free survival (DFS), objective response (OR) or overall survival (OS). The dosage regimen of a therapy described herein that is effective to treat a primary or a secondary hepatic cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Tumor” as it applies to a subject diagnosed with, or suspected of having, a primary or a secondary hepatic cancer, refers to a malignant or potentially malignant neoplasm or tissue mass of any size. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

### Methods, Uses and Medicaments

In one aspect, the invention relates to a method for treating cancer in an individual comprising administering to the individual a combination therapy which comprises: blinatumomab or a blinatumomab variant; and pembrolizumab, a pembrolizumab variant or an antigen-binding fragment thereof.

The combination therapy may also comprise one or more additional therapeutic agents. The additional therapeutic agent may be, e.g., a chemotherapeutic agent, a biotherapeutic agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF). The specific dosage and dosage schedule of the additional therapeutic agent can further vary, and the optimal dose, dosing schedule and route of administration will be determined based upon the specific therapeutic agent that is being used.

Examples of chemotherapeutic agents include alkylating agents such as thiotepa and cyclophosphamide; alkyl sulfonates such as busulfan, improsulfan and piposulfan; aziri dines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; cally statin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancrati statin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill, see, e.g., Agnew, Chem. Intl. Ed. Engl., 33: 183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes

(especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylormthine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene,
LYI 17018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy of the invention may be administered either alone or in the same medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a biotherapeutic that is administered at least daily and a biotherapeutic that is administered less frequently, such as once weekly, once every two weeks, or once every three weeks and/or are administered for different lengths of time, e.g., one therapeutic agent is administered IV for 30 minutes and one therapeutic agent is administered CIVI for a greater length of time than one hour.

In particularly preferred embodiments, blinatumomab or a blinatumomab variant is administered before administration of pembrolizumab, a pembrolizumab variant or an antigen-binding fragment thereof. In other particularly preferred embodiments, blinatumomab or a blinatumomab variant is administered concurrently with pembrolizumab, a pembrolizumab variant or an antigen-binding fragment thereof. In other embodiments, blinatumomab or a blinatumomab variant is administered after administration of pembrolizumab, a pembrolizumab variant or an antigen-binding fragment thereof.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is cancer treatment-naive. In other embodiments, the combination therapy is administered to a patient who failed to achieve a sustained response after prior therapy (e.g., after failed or ineffective therapy with a systemic anti-cancer therapy that is not blinatumomab/pembrolizumab combination therapy), i.e., is cancer treatment-experienced.

A combination therapy of the invention is typically used to treat a tumor that is large enough to be found by palpation or by imaging techniques well known in the art, such as MRI, ultrasound, or CAT scan.

Selecting a dosage regimen (also referred to herein as an administration regimen) for a combination therapy of the invention depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341: 1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343: 1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy. The optimal dose for blinatumomab in combination with pembrolizumab may be identified by dose escalation or dose de-escalation of one or both of these agents.

The present invention also provides a medicament which comprises blinatumomab and/or a blinatumomab variant, for use in treating DLBCL in a subject in combination with pembrolizumab, a pembrolizumab variant and/or an antigen-binding fragment thereof.

Further provided is a medicament which comprises pembrolizumab, a pembrolizumab variant and/or an antigen-binding fragment thereof, for use in treating DLBCL in a subject in combination with blinatumomab and/or a blinatumomab variant.

In some embodiments, a medicament comprising blinatumomab and/or a blinatumomab variant, or pembrolizumab, a pembrolizumab variant and/or an antigen-binding fragment thereof, as described above, may be provided as a liquid formulation or prepared by reconstituting a lyophilized powder with sterile water for injection prior to use.

In some embodiments, a medicament comprising blinatumomab is provided in a glass vial which contains a sterile, preservative-free, white to off-white, lyophilized powder for IV infusion following reconstitution with sterile water for injection. The reconstituted solution is added to an infusion bag containing 0.9% NaCl and a product-specific stabilizer (IV Solution Stabilizer). The IV Solution Stabilizer is supplied in 10 mL single-use glass injection vials as a sterile, preservative-free, clear, colorless-to-slightly-yellow liquid concentrate.

In some embodiments, a medicament comprising pembrolizumab is provided in a glass vial which contains about 100 mg of pembrolizumab in 4 mL of solution. Each 1 mL of solution contains 25 mg of pembrolizumab and is formulated in: L-histidine (1.55 mg), polysorbate 80 (0.2 mg), sucrose (70 mg), and water for injection, USP. The solution requires dilution for IV infusion.

Biotherapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 jpg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346: 1692-1698; Liu et al. (1999) J. Neurol. Neurosurg. Psych. 67: 451-456; Portielji et al. (20003) Cancer Immunol. Immunother. 52: 133-144.

In certain embodiments that employ pembrolizumab, a pembrolizumab variant and/or an antigen-binding fragment thereof, the dosing regimen will comprise administering pembrolizumab, a pembrolizumab variant and/or an antigen-binding fragment thereof at a dose of 1, 2, 3, 5 or 10 mg/kg at intervals of about 14 days (±2 days) or about 21 days (±2 days) or about 30 days (±2 days) throughout the course of treatment. In a preferred embodiment, pembrolizumab, a pembrolizumab variant or an antigen-binding fragment thereof is used at a dose of 200 mg (fixed) every 3 weeks.

In other embodiments that employ pembrolizumab, a pembrolizumab variant and/or an antigen-binding fragment thereof in the combination therapy, the dosing regimen will comprise administering pembrolizumab, a pembrolizumab variant and/or an antigen-binding fragment thereof at a dose of from about 0.005 mg/kg to about 10 mg/kg, with intra-patient dose escalation. In other escalating dose embodiments, the interval between doses will be progressively shortened, e.g., about 30 days (±3 days) between the first and second dose, about 21 days (±3 days) between the second and third doses. In certain embodiments, the dosing interval will be about 21 days (±3 days), for doses subsequent to the second dose.

In certain embodiments, a subject will be administered a parenteral dosing, e.g., an intravenous (IV) infusion, of a medicament comprising any of pembrolizumab, a pembrolizumab variant and/or an antigen-binding fragment thereof.

In a preferred embodiment of the invention, pembrolizumab, a pembrolizumab variant and/or an antigen-binding fragment thereof is administered in a liquid medicament at a dose selected from the group consisting of 1 mg/kg every two weeks (Q2W) or every 14 days (Q14D), 2 mg/kg Q2W or Q14D, 3 mg/kg Q2W or Q14D, 5 mg/kg Q2W or Q14D, 10 mg Q2W or Q14D, 1 mg/kg every three weeks (Q3W) or every 21 days (Q21D), 2 mg/kg Q3W or Q21D, 3 mg/kg Q3W or Q21D, 5 mg/kg Q3W or Q21D, 10 mg Q3W or Q21D, and flat-dose equivalents of any of these doses, i.e., such as 200 mg Q3W or Q21D.

In some embodiments, pembrolizumab, a pembrolizumab variant and/or an antigen-binding fragment thereof is provided in a dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg or about 400 mg.

In certain exemplary embodiments, pembrolizumab, a pembrolizumab variant and/or an antigen-binding fragment thereof is provided in a dosage of about 200 mg. In other exemplary embodiments, pembrolizumab, a pembrolizumab variant and/or an antigen-binding fragment thereof is provided as a liquid medicament which comprises 25 mg/ml pembrolizumab, 7% (w/v) sucrose, 0.02% (w/v) polysorbate 80 in 10 mM histidine buffer pH 5.5.

In some embodiments, the selected dose of pembrolizumab, a pembrolizumab variant and/or an antigen-binding fragment thereof is administered by IV infusion. In one embodiment, the selected dose of pembrolizumab, a pembrolizumab variant and/or an antigen-binding fragment thereof is administered by IV infusion over a time period of between 25 and 40 minutes, or about 30 minutes.

In certain embodiments, blinatumomab or a blinatumomab variant is administered for a first period of time (i.e., a “first treatment cycle”) and a second period of time (i.e., a “consolidation cycle”). Optionally, one or more additional consolidation cycles are administered, e.g., for a third period of time, a fourth period of time, a fifth period of time, etc. A time period between two treatment cycles wherein blinatumomab or a blinatumomab variant is not administered (e.g., the time between the first treatment cycle and a first consolidation cycle) is referred to as a “treatment-free” cycle.

In certain exemplary embodiments, it is envisaged that said first treatment cycle is at least about 14 days long, about 15 days long, about 16 days long, about 17 days long, about 18 days long, about 19 days long, about 20 days long, about 21 days long, about 22 days long, about 23 days long, about 24 days long, about 25 days long, about 26 days long, about 27 days long, about 28 days long, about 29 days long, about 30 days long, about 31 days long, about 32 days long, about 33 days long, about 34 days long, about 35 days long, about 36 days long, about 37 days long, about 38 days long, about 39 days long, about 40 days long, about 41 days long, about 42 days long, about 43 days long, about 44 days long, about 45 days long, about 46 days long, about 47 days long, about 48 days long, about 49 days long, about 50 days long, about 51 days long, about 52 days long, about 53 days long, about 54 days long, about 55 days long, about 56 days long, about 57 days long, about 58 days long, about 59 days long, about 60 days long, about 61 days long, about 62 days long, about 63 days long or longer.

In certain exemplary embodiments, it is envisaged that said first treatment cycle is between about 35 and about 77 days, between about 42 and about 70 days, between about 49 and about 63 days, between about 52 and about 60 days, or between about 54 and about 58 days or any number of days between these ranges.

In a particularly preferred embodiment, it is envisaged that said first treatment cycle is about 56 days.

In certain exemplary embodiments, it is envisaged that a consolidation cycle is at least about 2 days long, about 3 days long, about 4 days long, about 5 days long, about 6 days long, about 7 days long, about 8 days long, about 9 days long, about 10 days long, about 11 days long, about 12 days long, about 13 days long, about 14 days long, about 15 days long, about 16 days long, about 17 days long, about 18 days long, about 19 days long, about 20 days long, about 21 days long, about 22 days long, about 23 days long, about 24 days long, about 25 days long, about 26 days long, about 27 days long, about 28 days long, about 29 days long, about 30 days long, about 31 days long, about 32 days long, about 33 days long, about 34 days long or about 35 days long.

In certain exemplary embodiments, it is envisaged that a consolidation cycle is between about 7 and about 49 days, between about 14 and about 42 days, between about 21 and about 35 days, between about 23 and about 33 days, or between about 25 and about 31 days or any number of days between these ranges.

In a particularly preferred embodiment, it is envisaged that a consolidation cycle is about 28 days.

In certain exemplary embodiments, it is envisaged that a treatment-free cycle is at least about 2 days long, about 3 days long, about 4 days long, about 5 days long, about 6 days long, about 7 days long, about 8 days long, about 9 days long, about 10 days long, about 11 days long, about 12 days long, about 13 days long, about 14 days long, about 15 days long, about 16 days long, about 17 days long, about 18 days long, about 19 days long, about 20 days long, about 21 days long, about 22 days long, about 23 days long, about 24 days long, about 25 days long, about 26 days long, about 27 days long, about 28 days long, about 29 days long, about 30 days long, about 31 days long, about 32 days long, about 33 days long, about 34 days long or about 35 days long.

In certain exemplary embodiments, it is envisaged that a treatment-free cycle is between about 7 and about 49 days, between about 14 and about 42 days, between about 21 and about 35 days, between about 23 and about 33 days, or between about 25 and about 31 days or any number of days between these ranges.

In a particularly preferred embodiment, it is envisaged that a treatment-free cycle is about 28 days (+/−3 days).

In certain exemplary embodiments, blinatumomab and/or a blinatumomab variant is provided in an initial dose, and/or one or more escalation doses, and/or a maintenance dose. As used herein, an “initial dose” is the first dosage amount of blinatumomab and/or a blinatumomab variant, e.g., about 9 μg/d. As used herein, a “maintenance dose” is a dosage amount of blinatumomab and/or a blinatumomab variant that is administered later in time than an initial dose, and that is a greater dosage amount than the initial dose. For example, an initial dose may be about 9 μg/d, and a maintenance dose may be about 28 μg/d, about 56 μg/d or about 112 μg/d.

In certain exemplary embodiments, blinatumomab and/or a blinatumomab variant is provided to a subject as an initial dose, a maintenance dose, and one or more escalation doses.

As used herein, an “escalation dose” is a dosage that is greater than an initial dose, but is not the maintenance dose amount. In certain embodiments, an escalation dose is a dosage that is greater than the maintenance dose amount. In an exemplary embodiment, an escalation dose is a dosage that is smaller than the maintenance dose amount. For example, when the maintenance dose is about 56 μg/d or about 112 μg/d, the escalation dose may be about 28 μg/d.

In certain exemplary embodiments, an initial dose, an escalation dose and/or a maintenance dose may each be administered to a subject daily for a period of time, e.g., for about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 51 days, about 52 days, about 53 days, about 54 days, about 55 days or about 56 days.

In some embodiments, blinatumomab and/or a blinatumomab variant is provided in a dosage of about 1 μg per day, about 2 μg per day, about 3 μg per day, about 4 μg per day, about 5 μg per day, about 6 μg per day, about 7 μg per day, about 8 μg per day, about 9 μg per day, about 10 μg per day, about 11 μg per day, about 12 μg per day, about 13 μg per day, about 14 μg per day, about 15 μg per day, about 16 μg per day, about 17 μg per day, about 18 μg per day, about 19 μg per day, about 20 μg per day, about 21 μg per day, about 22 μg per day, about 23 μg per day, about 24 μg per day, about 25 μg per day, about 26 μg per day, about 27 μg per day, about 28 μg per day, about 29 μg per day, about 30 μg per day, about 31 μg per day, about 32 μg per day, about 33 μg per day, about 34 μg per day, about 35 μg per day, about 36 μg per day, about 37 μg per day, about 38 μg per day, about 39 μg per day, about 40 μg per day, about 41 μg per day, about 42 μg per day, about 43 μg per day, about 44 μg per day, about 45 μg per day, about 46 μg per day, about 47 μg per day, about 48 μg per day, about 49 μg per day, about 50 μg per day, about 51 μg per day, about 52 μg per day, about 53 μg per day, about 54 μg per day, about 55 μg per day, about 56 μg per day, about 57 μg per day, about 58 μg per day, about 59 μg per day, about 60 μg per day, about 61 μg per day, about 62 μg per day, about 63 μg per day, about 64 μg per day, about 65 μg per day, about 66 μg per day, about 67 μg per day, about 68 μg per day, about 69 μg per day, about 70 μg per day, about 71 μg per day, about 72 μg per day, about 73 μg per day, about 74 μg per day, about 75 μg per day, about 76 μg per day, about 77 μg per day, about 78 μg per day, about 79 μg per day, about 80 μg per day, about 81 μg per day, about 82 μg per day, about 83 μg per day, about 84 μg per day, about 85 μg per day, about 86 μg per day, about 87 μg per day, about 88 μg per day, about 89 μg per day, about 90 μg per day, about 91 μg per day, about 92 μg per day, about 93 μg per day, about 94 μg per day, about 95 μg per day, about 96 μg per day, about 97 μg per day, about 98 μg per day, about 99 μg per day, about 100 μg per day, about 110 μg per day, about 111 μg per day, about 112 μg per day, about 113 μg per day, about 114 μg per day, about 115 μg per day, about 116 μg per day, about 117 μg per day, about 118 μg per day, about 119 μg per day, about 120 μg per day, about 121 μg per day, about 122 μg per day, about 123 μg per day, about 124 μg per day, about 125 μg per day, about 126 μg per day, about 127 μg per day, about 128 μg per day, about 129 μg per day about 130 μg per day.

In certain exemplary embodiments, blinatumomab and/or a blinatumomab variant is provided in a dosage of between about 9 μg and about 112 μg per day. In other exemplary embodiments, blinatumomab and/or a blinatumomab variant is provided in a dosage of between about 9 μg and about 56 μg per day. In still other exemplary embodiments, blinatumomab and/or a blinatumomab variant is provided in a dosage of between about 9 μg and about 28 μg per day.

In certain exemplary embodiments that employ blinatumomab and/or a blinatumomab variant in a first treatment cycle and/or in one or more consolidation cycles, the dosing regimen comprises administering blinatumomab and/or a blinatumomab variant initially at a dose of about 9 μg/day, with intra-patient dose escalation at approximately 7-day intervals up to a maximal dose of about 28 μg/day, of about 56 μg/day or of about 112 μg/day. Once the maximal dose is reached, that dose is continued until the first treatment cycle or the first consolidation cycle is complete.

In certain embodiments, a subject will be administered a parenteral dosing, e.g., an intravenous (IV) infusion (e.g., via continuous intravenous infusion (CIVI)), of a medicament comprising blinatumomab and/or a blinatumomab variant.

In certain exemplary embodiments, blinatumomab and/or a blinatumomab variant is provided as 4 mL single-use glass injection vial containing a sterile, preservative-free, white to off-white, lyophilized powder for IV infusion following reconstitution with sterile water for injection. The standard commercial vial of blinatumomab is 35 μg (with a nominal fill of 38 μg). In a particular embodiment, the vial is reconstituted with 3 mL of sterile water (e.g., sterile water for irrigation) to provide a solution with a concentration of 12.5 μg/mL. The 12.5 μg/mL solution can then be further diluted to a concentration dependent on dose and final dosing volume prior to administration.

The reconstituted solution is added to an infusion bag containing 0.9% NaCl and a product-specific stabilizer (IV solution stabilizer). The IV solution stabilizer functions to prevent adsorption of blinatumomab to surfaces of the infusion components. The IV solution stabilizer is supplied in 10 mL single-use glass injection vials as a sterile, preservative-free, clear, colorless-to-slightly-yellow liquid concentrate.

In some embodiments, the selected dose of blinatumomab and/or a blinatumomab variant is administered by IV infusion, e.g., by CIVI. In one embodiment, the selected dose of blinatumomab and/or a blinatumomab variant is administered by CIVI over a time period of about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In a particularly exemplary embodiment, the selected dose of blinatumomab and/or a blinatumomab variant is administered by CIVI as a continuous infusion over a 24-hour period of time.

In certain exemplary embodiments, the patient is selected for treatment with the combination therapy of the invention if the patient has histologically confirmed DLBCL that is (1) refractory to first or later treatment; (2) is a first or later relapse and has received at least two prior therapies (one of which can be frontline therapy); or (3) has relapsed post-autologous hematopoietic stem cell transplantation (HSCT).

The medicaments described herein may be provided as a kit which comprises a first container and a second container and a package insert. The first container contains at least blinatumomab and/or a blinatumomab variant, and the second container contains at least one dose of a medicament comprising a pembrolizumab, a pembrolizumab variant and/or an antigen-binding fragment thereof. The kit can optionally comprise a package insert, or label, which includes instructions for treating a patient for cancer using the medicaments. The first and second containers may be comprised of the same or different shapes (e.g., vials, syringes and bottles) and/or materials (e.g., plastic or glass). The kit may further comprise other materials that may be useful in administering the medicaments, such as diluents, filters, IV bags and lines, infusion pumps, needles and syringes. In some preferred embodiments of the kit, the instructions state that the medicaments are intended for use in treating a patient having DLBCL.

### Pharmaceutical Compositions

The invention pertains to uses of the above-described agents for prophylactic and/or therapeutic treatments as described Infra. Accordingly, blinatumomab and/or a blinatumomab variant, and/or pembrolizumab, a pembrolizumab variant and/or an antigen-binding fragment thereof of the present invention can be incorporated into pharmaceutical compositions suitable for administration. Such compositions typically comprise blinatumomab and/or a blinatumomab variant or pembrolizumab, a pembrolizumab variant and/or an antigen-binding fragment thereof and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous, IS, ICV and/or IT administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

It is especially advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack or dispenser together with optional instructions for administration.

The pharmaceutical compositions of the present invention may be administered in a number of ways depending upon whether local or systemic treatment is desired and upon the area to be treated. Administration may be intratumoral or parenteral. Parenteral administration includes intravenous drip, subcutaneous, intraperitoneal or intramuscular injection, intrathecal, or intraventricular administration.

In one embodiment, unit doses or measured doses of a composition that include blinatumomab, blinatumomab variant, pembrolizumab, pembrolizumab variant and/or antigen-binding fragment thereof are dispensed by an implanted device. The device can include a sensor that monitors a parameter within a subject. For example, the device can include a pump, such as an osmotic pump and, optionally, associated electronics.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following example, which is included for purposes of illustration only and are not intended to be limiting. All patents, patent applications and references described herein are incorporated by reference in their entireties for all purposes.

### EXAMPLES

### Example 1. Phase Ib Open Label Study Investigating Safety and Efficacy of Blinatumomab in Combination with Pembrolizumab in Adult Subjects with Relapsed or Refractory Diffuse Large B Cell Lymphoma (DLBCL)

### Summary

The primary objective of the study is to determine the maximum tolerated dose (MTD) of blinatumomab in combination with pembrolizumab in adult subjects with relapsed or refractory (r/r) DLBCL. The secondary objectives of the study are to evaluate the safety, efficacy, and pharmacokinetics (PK) of blinatumomab in combination with pembrolizumab in adult subjects with r/r DLBCL.

The overlying hypothesis is that blinatumomab in combination with pembrolizumab will be tolerable in r/r DLBCL.

The primary endpoint is the incidence of dose limiting toxicities (DLTs). The secondary endpoints are: Overall response rate (ORR) by Cheson Criteria (2007); Complete response (CR) by Cheson Criteria; Duration of response (DOR) by ORR, CR, and partial response (PR); PFS; OS; Blinatumomab PK parameters; and Pembrolizumab PK parameters. The safety endpoints are the incidence and severity of adverse effects.

This is an open label, multicenter, phase 1b study testing the combination of blinatumomab with pembrolizumab in r/r DLBCL. The study will consist of 2 portions. Part 1 (n=6-50) will test the safety of up to 3 different blinatumomab target dose levels and up to 3 schedules of blinatumomab in combination with pembrolizumab in a rolling 6 design. (See Table 2.) A Dose Level Review Team (DLRT) will review the safety data to evaluate possible drug effects and DLTs. Subjects who are not on the dose ultimately selected for part 2 will remain on their initial dose throughout the study. Part 2 (n=36) will consist of an expansion cohort to assess PK, safety, and preliminary efficacy data at the chosen target dose and schedule. The part 2 dose will be determined by the totality of the clinical data from part 1 as determined by the DLRT.

TABLE 2

| Arms | Assigned Interventions |
|---|---|
| Experimental: COHORT Ib | Drug: Blinatumomab plus |
| Blinatumomab 9 to 28 microgram plus | Pembrolizumab |
| Pembrolizumab (day 1). | |
| Experimental: COHORT IIb | Drug: Blinatumomab plus |
| Blinatumomab 9 to 28 to 112 | Pembrolizumab |
| microgram plus Pembrolizumab (day 1). | |
| Experimental: COHORT IIIb | Drug: Blinatumomab plus |
| Blinatumomab 9 to 28 to 56 microgram | Pembrolizumab |
| plus Pembrolizumab (day 1). | |
| Experimental: COHORT Ia | Drug: Blinatumomab plus |
| Blinatumomab 9 to 28 microgram plus | Pembrolizumab |
| Pembrolizumab (day 15). | |
| Experimental: COHORT IIa | Drug: Blinatumomab plus |
| Blinatumomab 9 to 28 to 112 | Pembrolizumab |
| microgram plus Pembrolizumab (day | |
| 19). | |
| Experimental: COHORT IIIa | Drug: Blinatumomab plus |
| Blinatumomab 9 to 28 to 56 microgram | Pembrolizumab |
| plus Pembrolizumab (day 19). | |
| Experimental: Expansion Cohort | Drug: Blinatumomab plus |
| Using cohort design from previous | Pembrolizumab |
| cohorts where Maximum Tolerated | |
| Dose was found | |

The study design includes:
A 21-day screening period;
A standard (core) treatment period of blinatumomab (first cycle) of 8 weeks;
A second (consolidation) cycle of blinatumomab of 28 days after a 28-day (+3 days) blinatumomab treatment free period, that can be administered to subjects with stable disease (SD), PR, or CR;
Pembrolizumab treatment until disease progression or up to 35 cycles in the absence of disease progression:
   On study day 15 for subjects in cohort Ia
   OR
   On study day 1 for subjects in cohorts Ib, IIb, and IIIb
   OR
   On study day 19 for subjects in cohort Ha and IIIa; and
A safety follow-up visit after 30 days (+7 days) of last dose of each protocol specified therapy.

Follow-up for survival and collection of subsequent anticancer therapies will occur every 12 weeks (±28 days) for following blinatumomab safety follow up visit for up to approximately 24 months from the last dose of pembrolizumab. A maximum of 86 subjects will be enrolled.

Summary of Subject Eligibility Criteria: This study seeks to enroll adult subjects with histologically confirmed Diffuse Large B Cell Lymphoma that is either refractory to first or later treatment, or first or later relapse and has received at least 2 prior therapies (one of which can be frontline therapy), or relapsed post autologous HSCT with adequate organ function.

Subjects will be excluded if they have Richter's transformation (DLBCL arising in the setting of prior chronic lymphocytic leukemia) or Primary Mediastinal B cell Lymphoma (PMBCL) or have history or presence of clinically relevant central nervous system (CNS) pathology such as epilepsy, paresis, aphasia, stroke, severe brain injury, dementia, Parkinson's disease, cerebellar disease, organic brain syndrome, or psychosis or has evidence of active, non-infectious pneumonitis, or has a history of interstitial lung disease.

Blinatumomab is administered as a continuous intravenous infusion (CIVI). The first cycle of blinatumomab treatment is 8 weeks in duration followed by a 28-day (±3 days) blinatumomab treatment-free interval. The initial dose of blinatumomab will be 9 μg/day and will be dose escalated at weekly intervals until the target dose is reached. If a subject meets the requirements for continuing study therapy, they may receive another cycle ofblinatumomab (cycle 2 consolidation cycle) of 28 days duration after a 28-day (±3 days) treatment free interval. The consolidation cycle dosing will be the same as the first 28 days of cycle 1 of blinatumomab, starting at 9 jpg/day with weekly dose escalations until the target dose is reached.

Pembrolizumab 200 mg will be administered intravenously (IV) for 30 minutes every 3 weeks starting on study day 15 in cohort Ia, on study day 1 in cohorts Ib, IIb, and IIIb, on study day 19 in cohorts Ha and IIIa (3-week cycle).

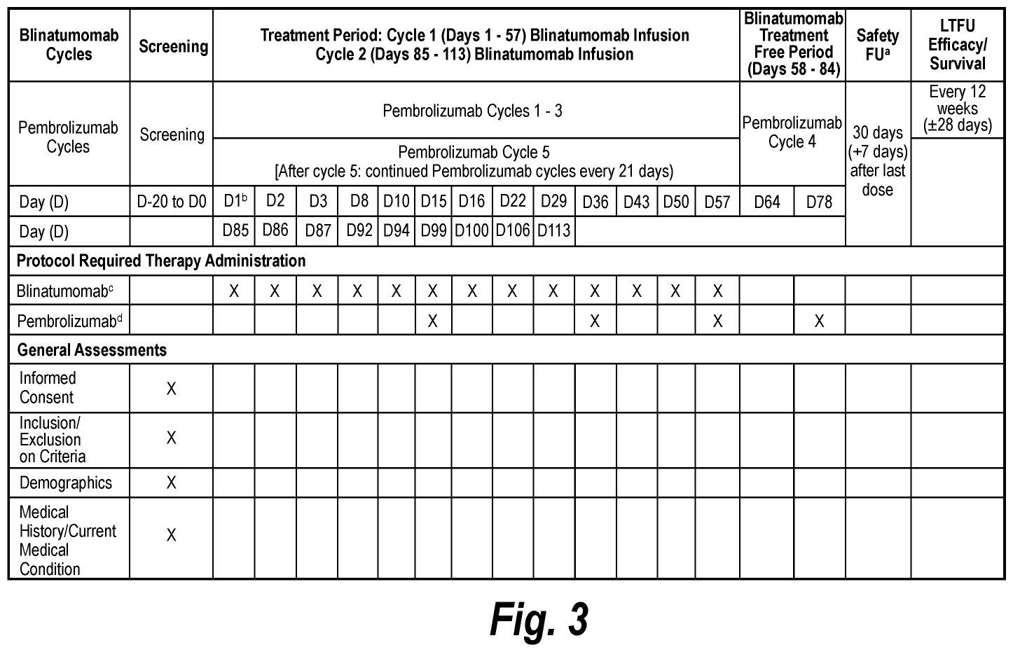

Written informed consent must be obtained from all subjects or legally acceptable representatives before any study specific procedures are performed. The following procedures will occur per the Schedule of Assessments: medical history, demographics, Eastern Cooperative Oncology Group (ECOG) performance status, neurological examination, physical exam including height, weight, vital signs, concomitant medications, adverse event/serious adverse event assessment, disease related events, and patient reported outcome (PRO) assessments. The subjects will undergo radiologic assessments (brain magnetic resonance imaging (MRI), computed tomography (CT) scan, and positron emission tomography (PET) scan) per the time points outlined in the Schedule of Assessments. Samples will be collected for local laboratory testing including: bone marrow biopsy, lumbar puncture, chemistry, coagulation, hematology (complete blood count (CBC)), immunoglobulins, urinalysis, thyroid function tests, creatinine clearance (CrCl), and pregnancy test. The subjects will further provide samples for central laboratory testing including: anti-blinatumomab antibodies, anti-pembrolizumab antibodies, immune panel, serum cytokines, PK (blinatumomab and pembrolizumab), core or excisional biopsy for biomarker analysis, PAXgene, and minimal residual disease (MRD) by next generation sequencing (NGS) as indicated in the Schedule of Assessments. A full list of study procedures, including the timing of each procedure, is described further below and is set forth at FIGS. 3-6.

Point estimates for efficacy endpoints will be accompanied by 2-sided 95% confidence intervals including estimates of Kaplan Meier (KM) quartiles, KM proportions, and binomial proportions. Pharmacokinetics will be performed by noncompartmental analysis. Pharmacodynamic samples will be summarized by descriptive statistics.

### Disease

Immunophenotyping is an essential diagnostic procedure which allows DLBCL to be identified and allows DLBCL to be further divided into germinal center (GC) type (cluster of differentiation (CD)10+ or CD10−, B-cell lymphoma 6 protein (BCL6)+ mouse monoclonal (MUM1−)) and non-GC type (CD10−, BCL6− or CD10−, BCL6+, MUM1+; Hans et al, 2004). Germinal center/non GC stratification by the Hans algorithm provides valuable prognostic information, but the supporting data is derived primarily from patients treated in the pre-rituximab era. Its prognostic value is less clear in patients treated with immunochemotherapy as opposed to cyclophosphamide, doxorubicin, vincristine, and prednisone (CHOP) alone (Nyman et al, 2007). Alternatively, prognostic differentiation can be achieved with gene expression profiling (Rosenwald et al, 2002) subdividing DLBCLs into GC types, activated B-cell (ABC) types and also Primary Mediastinal B-cell Lymphoma (PMBCL). The prognostic stratification between GC and ABC sub types remains valid in patients receiving immunochemotherapy (Lenz et al, 2008).

The GC-like lymphomas probably arise from normal GC B-cells and are associated with the t(14;18) translocation, deletion of phosphatase and tensin homolog (PTEN), amplification of the micro ribonucleic acid (RNA) cluster-17-92 (miR-17-92), and protein 53 (p53) mutations. The ABC Lymphomas are thought to originate from a post GC B cell and are characterized by activation of the nuclear factor kappa B (NFkB) and Janus Kinase (JAK) signalling pathways (Lenz and Staudt, 2010).

The International Prognostic Index (IPI) and age-adjusted IPI (aaIPI) have been developed as models for predicting outcomes based on clinical factors (The international NHL prognostic factors project, 1993) (Table 3).

TABLE 3
International Prognostic Index (IPI) for DLBCL. aaIPI =
age-adjusted International Prognostic Index; DLBCL = diffuse
large B-cell lymphoma; ECOG = Eastern Cooperative Oncology Group.

| IPI | | aaIPI | |
|---|---|---|---|
| Risk group | IPI Factors | Risk group | IPI Factors |
| Low | 0 or 1 | Low | 0 |
| Low Intermediate | 2 | Low Intermediate | 1 |
| High Intermediate | 3 | High Intermediate | 2 |
| High | 4 or 5 | High | 3 |

IPI Factors:
Older than 60 years of age (not used for aaIPI)
Disease stage III/IV
Lactate dehydrogenase level elevated
ECOG performance score ≥ 2
Extranodal disease > 1 site (not used for aaIPI)

The aaIPI is widely used for stratification and analysis of clinical trials. The data for the IPI derives from the pre-rituximab era, and when immunochemotherapy is used as first line treatment, the IPI appears less predictive in some series (Sehn et al, 2007) but not in others (Ziepert et al, 2010). A revised version has been developed in the post-rituximab era and is currently still under evaluation (Sehn et al, 2007).

### Rationale

Both PD-L1 and soluble PD-L1 expression has been reported in DLBCL and the expression of these ligands has been correlated with an inferior prognosis (Andorsky et al., 2011). Immune checkpoint inhibitors including pembrolizumab are being actively investigated in hematologic malignancies and have demonstrated single agent activity in lymphomas including DLBCL (Kiyasu et al, 2015; Lesokhin et al, 2016). The KEYNOTE 013 trial is currently testing pembrolizumab in a cohort of DLBCL subjects.

Furthermore, pre-clinical studies of blinatumomab have identified involvement of the PD1/PD-L1 axis as a potential mechanism of resistance to BiTE® mediated therapy. In r/r ALL, upregulation of PD-L1 has been observed on lymphoblasts of a patient receiving blinatumomab (Kohnke et al., 2015) and in vitro blockade of the PD-1/PD-L1 axis augmented lysis of acute myelogenous leukemia (AML) cells by the CD33/CD3 BiTE® antibody construct AMG 330 (Krupka et al., 2016). In line with these data, using AML cell lines engineered to ectopically overexpress individual T cell ligands, Lazlo et all demonstrated that the expression of PD L and PL L2 significantly reduced the anti-leukemic activity of AMG 330 (Lazlo et al, 2015). Similarly, Kenderian et al. (2016) showed that incubation of primary AML samples with either CD123 chimeric antigen receptor t-cell (CAR-T) or CD-33 CAR-T resulted in a significant upregulation of PD-1 on AML T cells and PD L1 on AML blasts. Using an AML xenograft model, they demonstrated that combination of a blocking PD-1 antibody plus CD-33 or CD-123 CAR-T enhanced the anti-leukemic activity of the single agent by significantly prolonging survival. In vitro dual blockade of PD-1 and PD-L1 with a CEA BiTE enhanced cytolytic activity of the BiTE on solid tumors (Osada et al., 2015). Finally, pediatric patients with ALL demonstrated increased expression of PD-L1 on leukemic blasts and combined treatment with blinatumomab and pembrolizumab was feasible and induced a response in a pediatric patient with ALL relapsed alloHSCT (Feuchtinger et al., 2015). Together, these data suggest that pembrolizumab could both unleash a polyclonal immune response against endogenous tumor antigen as well as enhance the CD-19 specific immune response elicited by blinatumomab, potentially leading to a synergistic effect.

### Pembroliumab Dose Selection

The dose of pembrolizumab planned to be studied in this trial is 200 mg Q3W. The dose recently approved in the United States and several other countries for treatment of melanoma subjects is 2 mg/kg Q3W. Information on the rationale for selecting 200 mg Q3W is summarized below.

In KEYNOTE-001, an open-label phase 1 study conducted to evaluate the safety, tolerability, PK and pharmacodynamics (PD), and antitumor activity of pembrolizumab when administered as monotherapy. The dose escalation portion of this trial evaluated three dose levels, 1 mg/kg, 3 mg/kg and 10 mg/kg, administered every 2 weeks (Q2W) and dose expansion cohorts evaluated 2 mg/kg Q3W and 10 mg/kg Q3W in subjects with advanced solid tumors. All dose levels were well tolerated and no dose-limiting toxicities were observed. This first-in-human study of pembrolizumab showed evidence of target engagement and objective evidence of tumor size reduction at all dose levels. No MTD has been identified. In addition, two randomized cohort evaluations of melanoma subjects receiving pembrolizumab at a dose of 2 mg/kg versus 10 mg/kg Q3W have been completed, and one randomized cohort evaluating 10 mg/kg Q3W versus 10 mg/kg Q2W has also been completed. The clinical efficacy and safety data demonstrate a lack of important differences in efficacy or safety profile across doses.

An integrated body of evidence suggests that 200 mg every 3 weeks (Q3W) is expected to provide similar response to 2 mg/kg Q3W, 10 mg/kg Q3W and 10 mg/kg Q2W. Previously, a flat pembrolizumab exposure-response relationship for efficacy and safety has been found in subjects with melanoma in the range of doses between 2 mg/kg and 10 mg/kg. Exposures for 200 mg Q3W are expected to lie within this range and will be close to those obtained with 2 mg/kg Q3W dose.

A population pharmacokinetic (PK) model, which characterized the influence of body weight and other patient covariates on exposure, has been developed. The PK profile of pembrolizumab is consistent with that of other humanized monoclonal antibodies, which typically have a low clearance and a limited volume of distribution. The distribution of exposures from the 200 mg fixed dose are predicted to considerably overlap those obtained with the 2 mg/kg dose and importantly will maintain individual patient exposures within the exposure range established in melanoma as associated with maximal clinical response. Pharmacokinetic properties of pembrolizumab, and specifically the weight-dependency in clearance and volume of distribution are consistent with no meaningful advantage to weight-based dosing relative to fixed dosing.

In translating to other tumor indications, similarly flat exposure-response relationships for efficacy and safety as observed in subjects with melanoma can be expected, as the antitumor effect of pembrolizumab is driven through immune system activation rather than through a direct interaction with tumor cells, rendering it independent of the specific tumor type. In addition, available PK results in subjects with melanoma, NSCLC, and other tumor types support a lack of meaningful difference in pharmacokinetic exposures obtained at tested doses among tumor types. Thus the 200 mg Q3W fixed-dose regimen is considered an appropriate fixed dose for other tumor indications as well.

A fixed dose regimen will simplify the dosing regimen to be more convenient for physicians and to reduce potential for dosing errors. A fixed dosing scheme will also reduce complexity in the logistical chain at treatment facilities and reduce wastage. The existing data suggest 200 mg Q3W as the appropriate dose for pembrolizumab.

### Blinatumomab Dose Selection

Three target doses will potentially be tested in part 1 in a dose-escalation design starting at the lowest blinatumomab target dose of 28 μg/day with the primary focus on identifying a safe combination dose. Blinatumomab will be escalated in a stepwise manner until the appropriate target dose is reached. This dosing paradigm is based on safety and efficacy data from the phase 1 Study MT103-104 in NHL (including DLBCL) and the phase 2 Study MT103-208 in DLBCL in which blinatumomab was tested as a monotherapy.

Step dosing of blinatumomab has been implemented to mitigate the potential for adverse events associated with excessive T cell activation and cytokine release. Blinatumomab has been associated with transient elevation of serum cytokines, especially IL-6, IL-10, and IFN-γ, the cytokine elevation largely occurred within the first two days following the initial dose of blinatumomab (Armand et al, 2013).

Accordingly, adverse events potentially related to T-cell activation and cytokine release, such as cytokine release syndrome (CRS) and neurologic events are more frequent at the time of initiation of blinatumomab treatment. Step-wise dosing has been shown to attenuate the cytokine release and reduce the occurrence/severity of those events in previous studies (MT103-104 and MT103-208).

In the MT103-208 study, grade 3 or higher neurologic treatment emergent adverse events (TEAEs) were reported in 21.7% of subjects receiving stepwise dosing and 100% of subjects receiving flat dosing with a median time to onset of 18 days. No CRS was reported in MT103-208, however grade 3 CRS was reported in 2% of subjects on Study MT103-211 in r/r ALL with a median time to onset of 2 days.

Part 2 will consist of an expansion cohort to ensure adequate safety and PK data is collected. The blinatumomab target dose will be based on safety data from part 1.

To minimize the risk of CRS and neurologic events, all patients will receive prophylactic dexamethasone for each blinatumomab infusion start and dose increase: 20 mg orally at 6 to 12 hours and 1 hour prior to infusion. In case of signs of CRS, dexamethasone will be given 8 mg orally 3 times daily for up to 72 hours.

### Design and Blinatumomab Escalation/De-Escalation Rules

Part 1

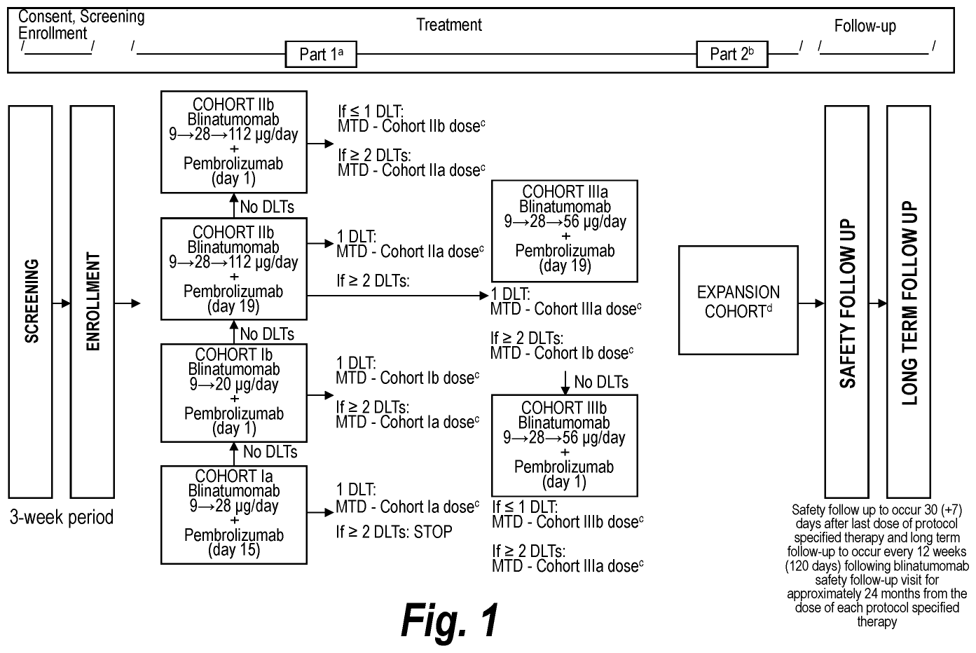

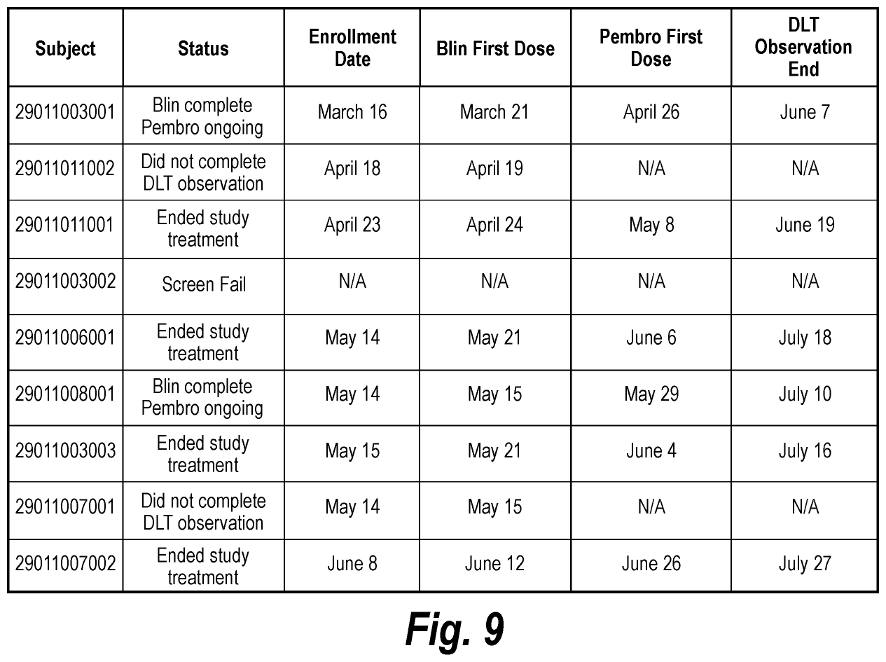

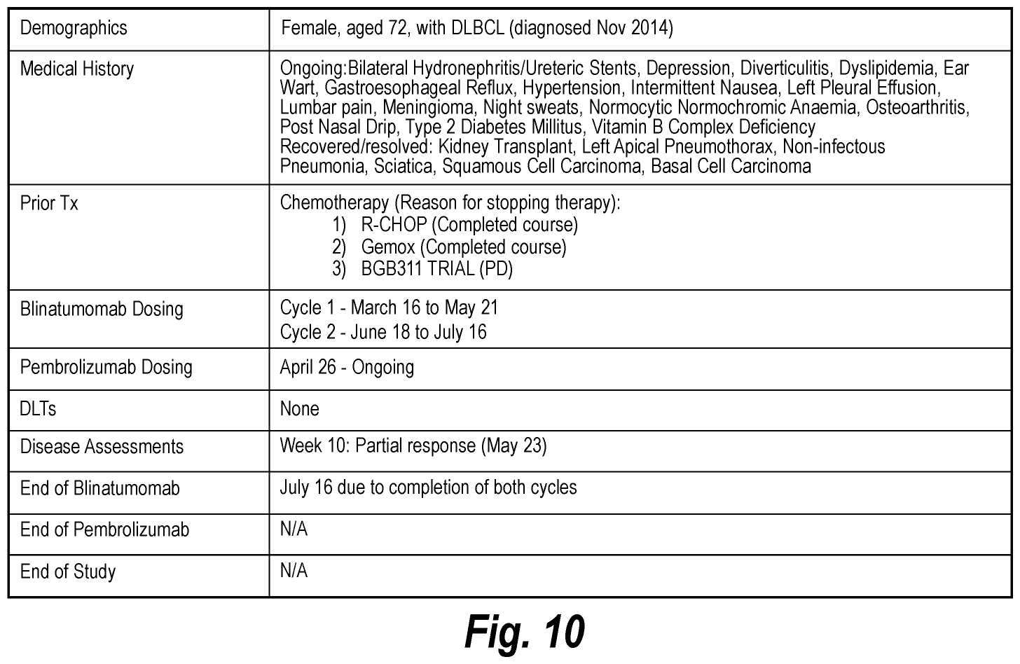

For part 1, subject enrollment in cohort 1a is outlined in the schema in FIG. 1. Blinatumomab was dosed as a continuous intravenous infusion (CIVI) for 8 weeks. The initial dose was 9 μg/day and the dose was escalated after 7 days to a target dose of 28 μg/day. The status overview of cohort 1a is shown at FIG. 9. A single subject overview (cohort 1a) is shown at FIG. 10.

Depending on tolerability, the target dose of blinatumomab will be increased to a maximum of 112 μg/day in cohort IIa and IIb, with possible de-escalation to 56 μg/day in cohorts IIIa and IIIb. Pembrolizumab was dosed by intravenous (IV) infusion 200 mg at Q3W starting on study day 15 in cohort Ia, will be started on study day 1 in cohorts Ib, IIb, and IIIb, and will be started on study day 19 in cohorts IIa and IIIa.

Subjects who do not meet the criteria for investigational product (IP) discontinuation are eligible for a second cycle of blinatumomab (consolidation) consisting of a CIVI of 28 days after a 28-day (+3 days) blinatumomab treatment-free interval. Blinatumomab will be started at 9 μg/day and escalated every 7 days to the maximum target dose of blinatumomab in the assigned cohort.

Subjects will be enrolled to part 1 with up to 6 subjects being enrolled per cohort. In any cohort, assuming adequate tolerability (≤1 DLT), up to 10 subjects may be enrolled to ensure adequate safety and PK data is collected. The decision to expand a cohort will be made by the DLRT.

The MTD of blinatumomab will be defined as the dose level at which at most 1 of 6 subjects experiences a DLT or the maximum administered dose (MAD). The MAD to be tested will be 112 μg/day (cohort IIa and IIb). The MTD defines the stopping rules for the study. Subjects who discontinue treatment prior to reaching the target dose in part 1 will be replaced.

The DLRT will review the available data in part 1 to determine if blinatumomab is safe and tolerable as defined by DLT criteria, taking into account the general risk:benefit ratio. The DLRT will meet when any of the following criteria are met: two or more subjects have experienced a DLT in a cohort; six subjects are enrolled in a cohort and all subjects have completed the DLT observation period; and in the event that a cohort is expanded to 10, DLRT may also meet after all subjects have completed DLT observation period.

Based on the totality of the clinical data, the DLRT may recommend to expand a cohort to a maximum of 10 subjects if the collection of more data is deemed warranted.

Part 2

For part 2, the dosing will be determined based on the safety of the combination of blinatumomab and pembrolizumab and the MTD of blinatumomab established in part 1 per DLRT. Part 2 will consist of an expansion cohort to collect further safety and PK data as well as provide a preliminary estimate of the efficacy of the combination of blinatumomab and pembrolizumab. Dose limiting toxicities will be monitored to ensure they do not reach a pre-defined threshold of 25%. If this threshold is reached, the DLRT will have the discretion to change to another dose/schedule tested in phase 1 part 1 based on the totality of the available data. The details of DLT boundaries and study endpoints are discussed below.

### Inclusion Criteria

In order to be eligible for participation in this trial, the subject must meet the following criteria: subject has provided written informed consent prior to initiation of any study specific procedures; age≥18 years at the time of informed consent; have histologically confirmed DLBCL that is either refractory to first or later treatment, or a first or later relapse AND has received at least 2 prior therapies (one of which can be frontline therapy) or elapsed post-autologous HSCT; have measurable disease defined as at least 1 lesion that can be accurately measured in at least 2 dimensions with spiral computerized tomography (CT) scan (minimum measurement must be either >15 mm in the longest diameter OR >10 mm in the short axis); demonstrate adequate organ function; have resolution of toxic effect(s) of the most recent prior chemotherapy to grade 1 or less (except alopecia) (if subject received major surgery or radiation therapy of >30 Gy, they must have recovered from the toxicity and/or complications from the intervention); female subjects of childbearing potential must have a negative urine or serum pregnancy test within 72 hours prior to receiving the first dose of study medication (if the urine test is positive or cannot be confirmed as negative, a serum pregnancy test will be required); female subjects of childbearing potential must be willing to use an adequate method of contraception for the course of the study through 120 days after the last dose of study medication (abstinence is acceptable if this is the usual lifestyle and preferred contraception for the subject); male subjects of childbearing potential must agree to use an adequate method of contraception starting with the first dose of study therapy through 120 days after the last dose of study therapy (abstinence is acceptable if this is the usual lifestyle and preferred contraception for the subject); Eastern cooperative oncology group (ECOG) performance status ≤2; life expectancy of ≥12 weeks in the opinion of the Investigator; and the subject must be able to provide an evaluable core or excisional biopsy prior to the start of treatment (for refractory disease, biopsy tissue collected up to 3 months prior to the first day of study treatment is acceptable; for relapsed disease, biopsy collected up to 28 days prior to the first day of study treatment is acceptable).

### Exclusion Criteria

Subjects meeting any of the following exclusion criteria will not be eligible to participate in this study: Richter's transformation (DLBCL arising in the setting of prior chronic lymphocytic leukemia) or PMBCL; has a history or presence of clinically relevant CNS pathology such as epilepsy, paresis, aphasia, stroke, severe brain injury, dementia, Parkinson's disease, cerebellar disease, organic brain syndrome, or psychosis; has disease that is suitable for local therapy administered with curative intent; is currently receiving treatment in another investigational device or drug study, or less than 30 days since ending treatment on another investigational device or drug study(s). Thirty days is calculated from day 1 of protocol-specified therapy; has a diagnosis of immunodeficiency or is receiving systemic steroid therapy (in dosing exceeding 10 mg daily of prednisone equivalent) or any other form of immunosuppressive therapy within 7 days prior to the first dose of protocol specified therapy (the use of physiologic doses of corticosteroids may be approved after consultation with the sponsor); has had a prior anti-cancer monoclonal antibody administered within 30 days prior to the first day of study treatment or who has not recovered (i.e., ≤grade 1 or at baseline) from adverse events due to agents administered more than 28 days earlier; has had prior chemotherapy, targeted small molecule therapy, or radiation therapy within 14 days prior to first day of study treatment or who has not recovered (i.e., ≤grade 1 or at baseline) from adverse events due to a previously administered agent (subjects with ≤grade 2 neuropathy or ≤grade 2 alopecia are an exception to this criterion and may qualify for the study); has undergone prior allogeneic HSCT within the last 5 years or greater than 5 years ago but has active graft versus host disease (GvHD) requiring systemic treatment; has received autologous HSCT within 6 weeks prior to start of treatment; has required transfusion of blood products (including platelets or red blood cells) or administration of colony stimulating factors (including granulocyte-stimulating factors, granulocyte macrophage-colony stimulating factors, or recombinant erythropoietin) within 14 days prior to first day of study treatment; has a history of other malignancy within the past 3 years with the exception of malignancy treated with curative intent and with no known active disease present for ≥3 years before enrollment and felt to be at low risk for recurrence by the treating physician, adequately treated non-melanoma skin cancer or lentigo maligna without evidence of disease, adequately treated cervical carcinoma in situ without evidence of disease, adequately treated breast ductal carcinoma in situ without evidence of disease, prostatic intraepithelial neoplasia without evidence of prostate cancer, or adequately treated urothelial papillary noninvasive carcinoma or carcinoma in situ; has known active CNS metastases and/or carcinomatous meningitis (subjects with previously treated brain metastases may participate provided they are stable (without evidence of progression by imaging (using the identical imaging modality for each assessment, either magnetic resonance imaging (MRI) or CT scan) for at least 28 days prior to the first dose of trial treatment and any neurologic symptoms have returned to baseline), have no evidence of new or enlarging brain metastases, and are not using steroids for at least 7 days prior to protocol specified therapy—this exception does not include carcinomatous meningitis which is excluded regardless of clinical stability); has active autoimmune disease that has required systemic treatment in past 2 years (i.e., with use of disease modifying agents, corticosteroids or immunosuppressive drugs) (replacement therapy (e.g., thyroxine, insulin, or physiologic corticosteroid replacement therapy for adrenal or pituitary insufficiency, etc.) is not considered a form of systemic treatment); has a history of (non-infectious) pneumonitis that required steroids or current pneumonitis; has a history of interstitial lung disease; has an uncontrolled active infection requiring systemic therapy; is pregnant or breastfeeding, or expecting to conceive or father children within the projected duration of the trial, starting with the screening visit through 120 days after the last dose of trial treatment; has received prior therapy with an anti-PD-1, anti-PD-L1, or anti-PD-L2 agent or if the subject has previously participated in Merck MK-3475 (pembrolizumab) clinical trials; has received prior anti-CD19 directed therapy; has a known hypersensitivity to immunoglobulins or any other component of the study drugs formulation; has a known history of human immunodeficiency virus (HIV) (HIV 1 and/or HIV 2 antibodies); has known active hepatitis B (e.g., hepatitis B antigen (HBsAg) reactive) or hepatitis C (e.g., HCV RNA (qualitative) is detected); has received a live vaccine within 30 days of planned start of protocol specified therapy; the subject is likely to not be available to complete all protocol required study visits or procedures, and/or to comply with all required study procedures to the best of the subject's and investigator's knowledge; or a history or evidence of any other clinically significant disorder, condition or disease (with the exception of those outlined above) that, in the opinion of the investigator or physician, if consulted, would pose a risk to subject safety or interfere with the study evaluation, procedures or completion.

### Treatment Procedures

Blinatumomab will be supplied as 4 mL single-use glass injection vials containing a sterile, preservative-free, white to off-white, lyophilized powder for IV infusion following reconstitution with sterile water for injection. Sterile water for injection and supplies required for reconstitution and injection of blinatumomab will not be provided to clinical sites.

To prepare blinatumomab for continuous intravenous infusion (CIVI), the lyophilized powder is reconstituted with sterile water for injection. The reconstituted solution is added to an infusion bag containing 0.9% NaCl and a product-specific stabilizer (IV Solution Stabilizer). The IV solution stabilizer functions to prevent adsorption of blinatumomab to surfaces of the infusion components. The IV Solution Stabilizer is supplied in 10 mL single-use glass injection vials as a sterile, preservative-free, clear, colorless-to-slightly-yellow liquid concentrate.

Blinatumomab is administered as a CIVI. The infusion bags will be changed by site nursing or home health care personnel trained on the protocol and on the proper administration of blinatumomab. The first cycle of blinatumomab treatment is 8 weeks in duration (See FIG. 1).

The first cycle is followed by a 28-day (±3 days) blinatumomab treatment-free interval. Those subjects who do not meet criteria for discontinuation after the blinatumomab treatment-free interval may then receive a consolidation cycle of blinatumomab (cycle 2) of 28 days duration. In both cycle 1 and the consolidation cycle, the initial dose of blinatumomab will be 9 μg/day and will be dose escalated at 7-day intervals until the target dose is reached. The dosing and schedule is outlined below.

The drug administration should not be interrupted, if possible. In case of infusion interruption, due to any technical or logistic reason, the interruption should be as short as possible and the infusion continued at the earliest time possible. Every interruption longer than 1 hour should be documented. Administration of dexamethasone premedication will occur as described below. If the infusion is interrupted, if possible, the total infusion time should equal 56 days in the first cycle or 28 days in the second cycle.

A dose of up to 10% higher than the intended blinatumomab dose (per day) may not require specific intervention. In case of overdose or medication error, the infusion should be immediately stopped. Routine supportive and symptomatic care according to standard medical practice is recommended. Once the subject is stabilized and no clinically relevant safety findings due to blinatumomab are observed, resumption of blinatumomab at a correct dose can be considered after consultation with the Amgen medical monitor.

For blinatumomab, a dose of greater than 10% higher than the intended dose will be considered clinically important and classified as a serious adverse event under the criterion of “other medically important serious event” If the overdose results in additional adverse event/s, the subject should be followed carefully until all signs of toxicity are resolved and the adverse event/s should be recorded/reported per Section 9 of the protocol.

The dose, start and stop date/time, and lot number of protocol-specified therapy is to be recorded on each subject's CRF. The date and time of infusion bag changes, all infusion start and stop times, and any dose modifications should also be recorded accurately.

Subjects who have been dose reduced will have an option to re-escalate to higher dose levels within their assigned dose cohort once the adverse event has resolved to grade 1 or less for at least 7 days.

Re-start of the infusion should be performed in the hospital, under supervision of the investigator. Before blinatumomab is re-started, premedication with dexamethasone must be administered as described in Table 7. The subject should be observed over night for possible side effects after the restart, either in the hospital or in the outpatient setting, as applicable.

In addition to the events described above, the dose may be temporarily or permanently reduced if, by investigator's judgment, it is necessary for safety reasons.

After at least 7 days of dosing at the reduced level, the dose may be increased back to the next higher dose level. An infusion interruption of more than 14 days due to an adverse event related to blinatumomab will lead to permanent discontinuation of treatment. In case of logistical difficulties, restart of treatment can be postponed for up to 7 additional days without resulting in permanent treatment discontinuation. Treatment may be also interrupted or permanently discontinued at the discretion of the investigator if any clinical/laboratory adverse event is considered to be medically relevant.

In case of signs of cytokine release, dexamethasone must be administered orally or IV at a dose of at maximum 3×8 mg/day for up to 72 hours.

Pembrolizumab Dosage. Administration, and Schedule

Trial treatment should begin as close as possible to the date on which the subject is allocated/assigned. The pembrolizumab treatment to be used in this trial is outlined below in Table 4.

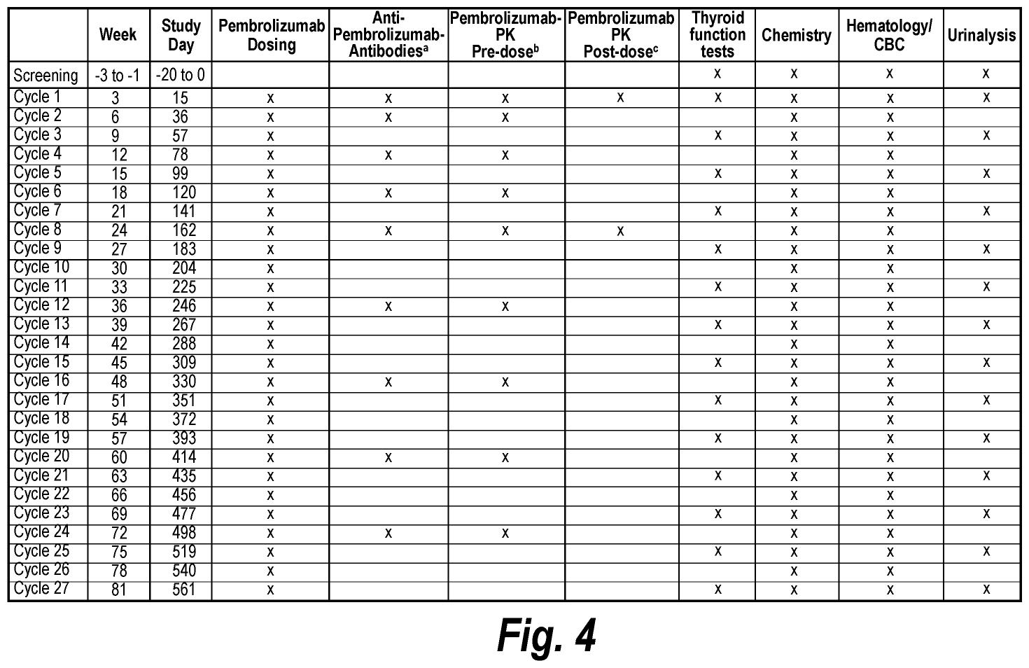

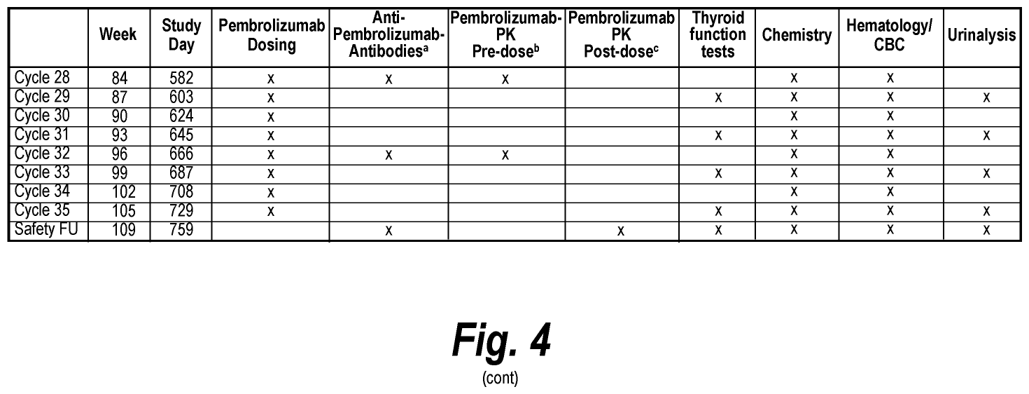

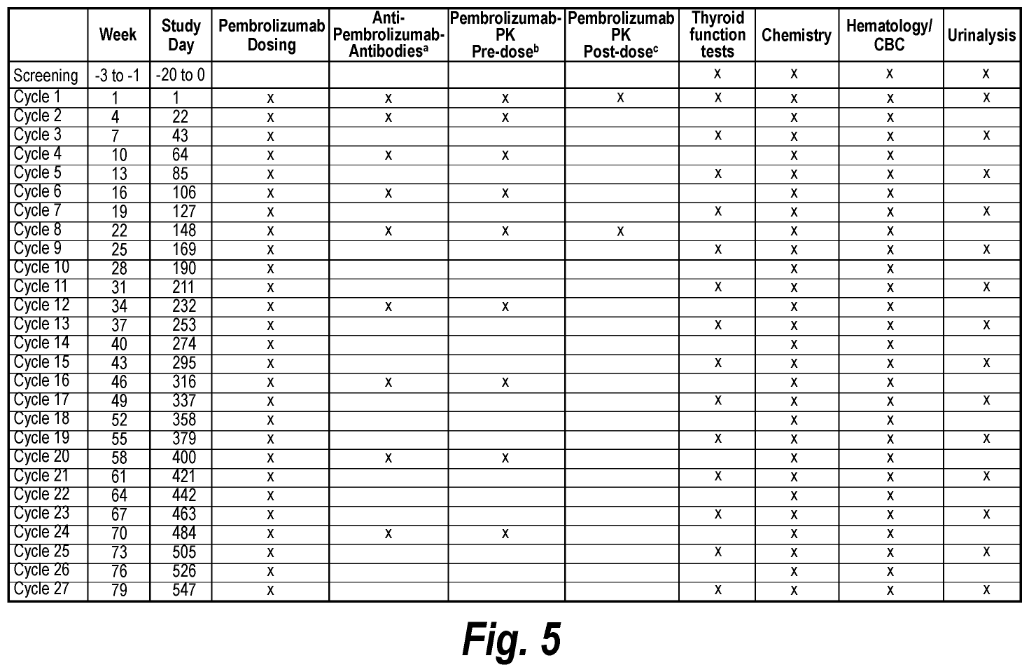

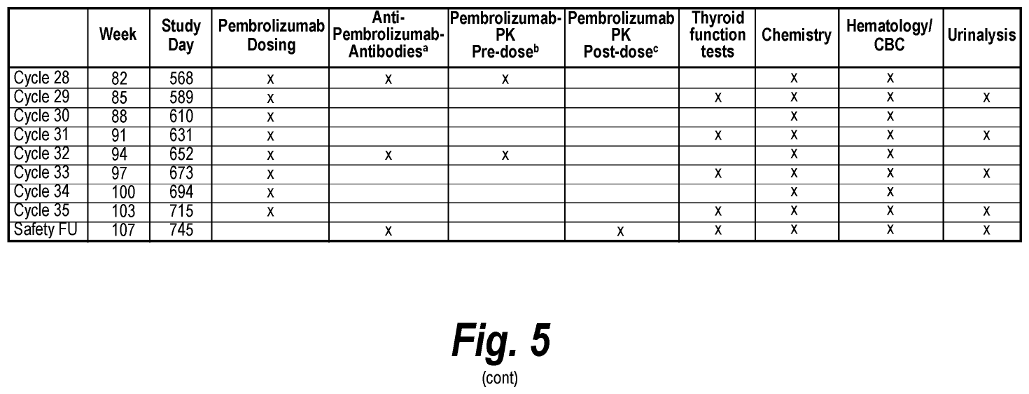

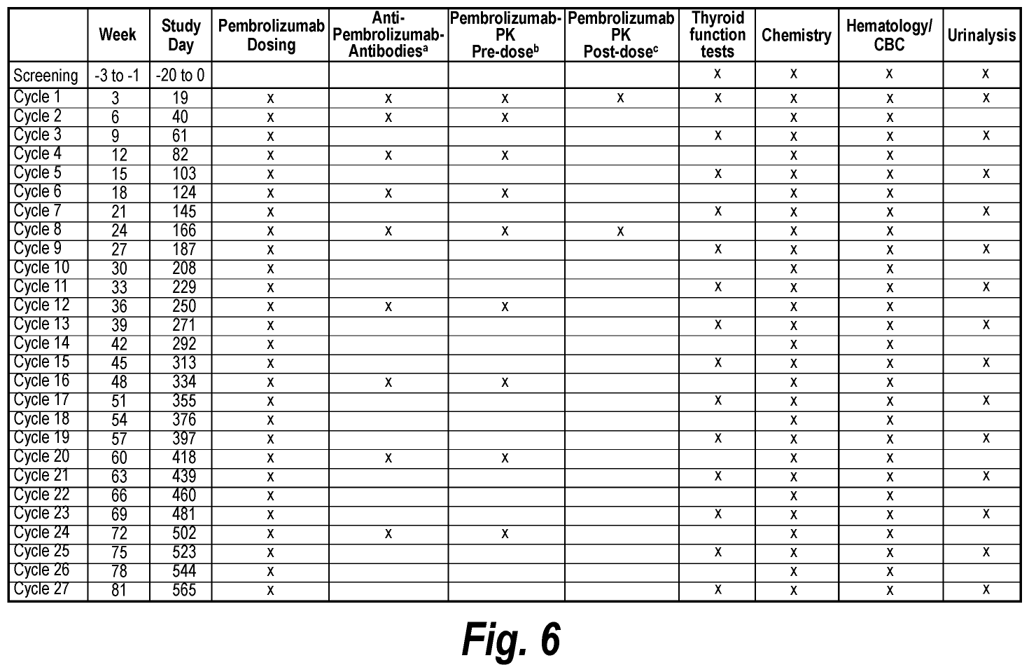

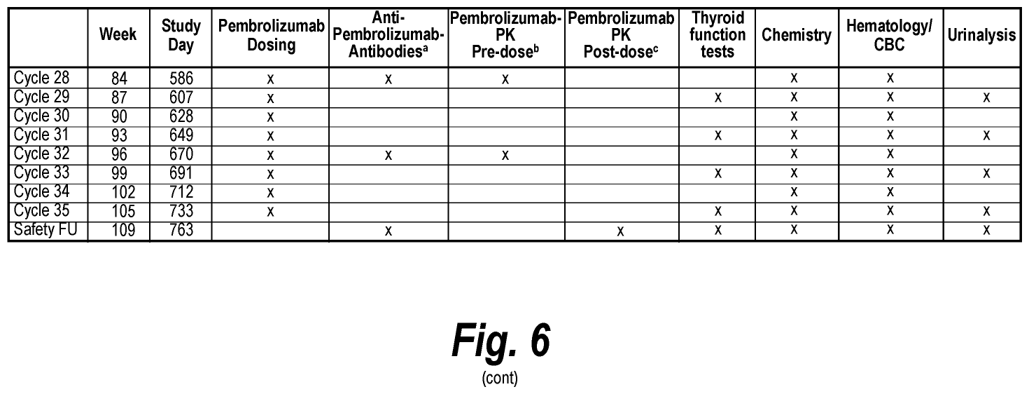

Schedule of pembrolizumab dosing and related assessments for cohort Ia are provided in FIGS. 3 and 4, for cohorts Ib, IIb, and IIIb are provided in FIG. 5, and for cohorts IIa and IIIa are provided in FIG. 6. Pembrolizumab will be administered as a dose of 200 mg using a 30-minute IV infusion. Sites should make every effort to target infusion timing to be as close to 30 minutes as possible. However, given the variability of infusion pumps from site to site, a window between −5 minutes and +10 minutes is permitted (i.e., infusion time is 30 minutes−5 minutes/+10 minutes).

For this trial, an overdose of pembrolizumab will be defined as 1000 mg (5 times the dose) of pembrolizumab. No specific information is available on the treatment of overdose of pembrolizumab. In the event of overdose of pembrolizumab, the subject should be observed closely for signs of toxicity. Appropriate supportive treatment should be provided if clinically indicated.

TABLE 4
Trial Treatment

| | | | Maximum | | | |
|---|---|---|---|---|---|---|
| | Dose/ | Dose | Length of | Route of | | |
| Drug | Potency | Frequency | Dosing | Administration | Regimen | Use |
| Pembrolizumab | 200 mg | Every | Up to | Intravenous | Day 1 of each cycles) | Experimental |
| | | 21 days | 35 cycles | | Starting at study | |
| | | | | | day 15 (cohort Ia), | |
| | | | | | study day 1 | |
| | | | | | (cohorts Ib, IIb, and | |
| | | | | | IIIb), and study day 19 | |
| | | | | | (cohorts IIa and IIIa) | |
| | | | | | (21-day cycles) | |

Mandatory premedication with dexamethasone is required 6 to 12 hours and 1 hour before each treatment cycle and dose step for the prevention of CRS resulting from blinatumomab treatment. Dexamethasone premedication will also be required before restarting blinatumomab after a dose interruption due to an adverse event or technical/logistical issue. Refer to Table 5 for details.

TABLE 5
Dexamethasone Pre-does Treatment and Events.

| Treatment Phase | Target Subjects: | Dexamethasone Dose |
|---|---|---|
| Pre-dose | All subjects | Dexamethasone 20 mg IV: |
| Dexamethasone | | within 1 hour prior |
| Prior to Each | | to start of treatment |
| Blinatumomab Treatment | | in each treatment |
| Cycle and Before Each | | cycle, and within |
| Dose Step Increase | | 1 hour prior to dose |
| | | step (increase). |
| Infusion Interruption/ | Subjects who | Dexamethasone 20 mg IV: |
| Dose Modification Due | interrupt | within 1 hour prior |
| to Adverse Event or | treatment > | to re-start of treatment |
| Interruption due to | 4 hours | |
| Technical/Logistical | | |
| Event | | |
| In case of signs of | Subjects with | Dexamethasone orally or |
| CRS | signs of CRS | IV at a dose maximum of |
| | | 3 doses of 8 mg/day |
| | | (24 mg/day) for up to |
| | | 72 hours. The dose should |
| | | then be reduced step-wise |
| | | over 4 days. |
| Infusion | Subjects with | Dexamethasone should be |
| Interruption/Dose | neurologic | administered at a dose |
| Modification Due to | event | of at least 24 mg/day for |
| Neurologic Events | | up to 72 hours. |
| | | Dexamethasone will then |
| | | be reduced step-wise |
| | | over 4 days. |

Blinatumomab must be administered using infusion pumps approved for use by the appropriate regulatory authorities for the country in which the subject is undergoing treatment. Blinatumomab infusion for solution will be prepared in bags for IV infusion and delivered through infusion lines that are both compatible with the investigational product as described in the IPIM. The blinatumomab final solution for infusion should not come into contact with the pump at any time.

### Study Procedures

Schedule of Assessments

FIGS. 3-6 depict outlines of the procedures required at each visit.

Criteria for Assessment of Disease

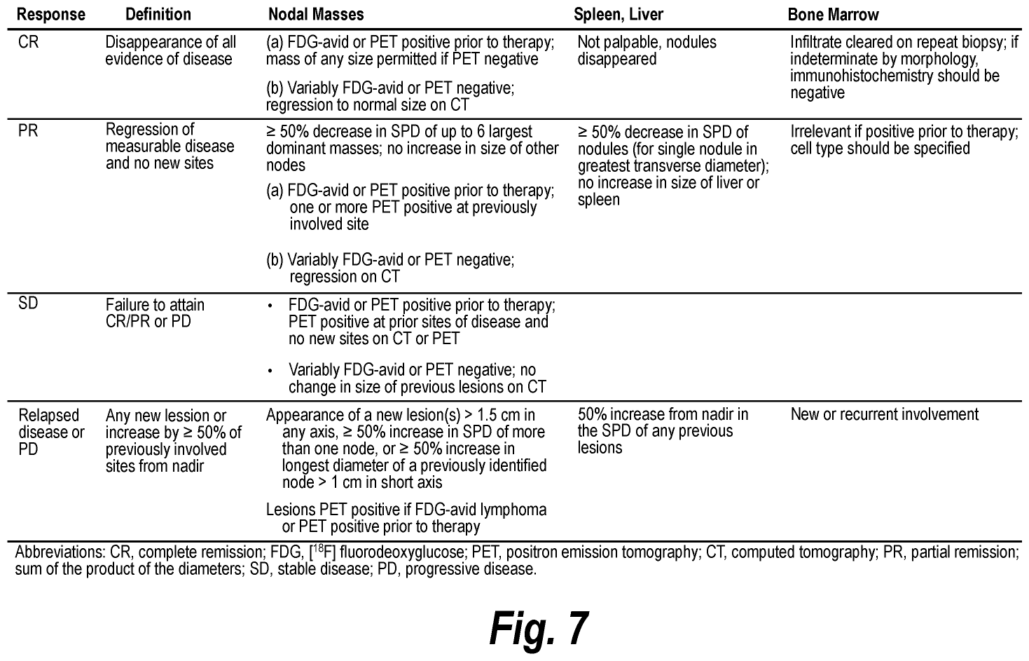

Antitumor activity will be evaluated using the Revised Response Criteria for Malignant Lymphoma criteria (Cheson et al, 2007) (FIG. 7). The International Working Group criteria will be applied by the site as the primary measure for assessment of disease response and as a basis for all protocol guidelines related to disease status (e.g., discontinuation of study therapy).

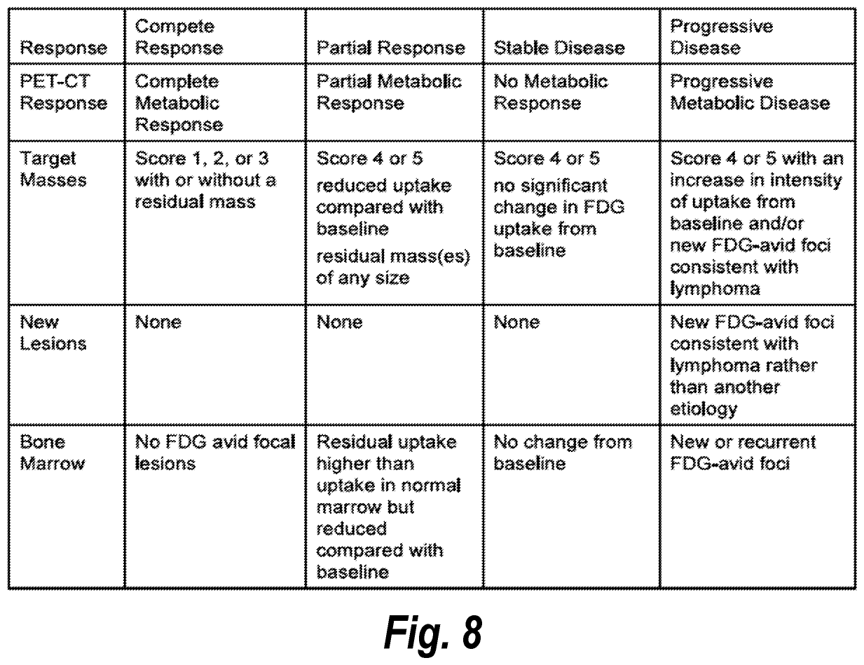

Antitumor activity will also be evaluated by independent central review as part of the exploratory analyses using Lugano Classification (Cheson et al, 2014). Lymphoma response assessment by CT/PET is based on the International Working Group response criteria for malignant lymphoma (Cheson et al, 2007). Local reading using Cheson classification (investigator assessment with site radiology reading) will be used to determine subject eligibility and for subject management. The sponsor will also receive radiologic images and a retrospective analysis of subject eligibility and treatment response may be performed by a central vendor. The central vendor will assess lymphoma response using both the Lugano and Cheson classification. Assessment of lymphoma B symptoms should occur with each lymphoma disease response assessment (FIG. 8).

### Pharmacokinetic Assessments

Blinatumomab

Pharmacokinetic (PK) assessments will be required for all subjects receiving blinatumomab. In cohorts Ia, Ib, IIb, and IIIb, blinatumomab samples will be collected at day 1 (pre-dose, 4, 6, 8 h after start of 9 μg/d infusion), day 2 (any time), day 8 (6-10 h after start of 28 μg/d infusion), day 10 (any time), day 15 (6-10 h after start of 112 μg/d infusion in cohort IIb or 56 μg/d in cohort IIIb or any time if 28 μg/d dose was continuously administered in cohort Ib, or 1 hour after pembrolizumab infusion has ended in cohort Ia), day 22 (any time), day 29 (any time) and day 43 (any time) in cycle 1. In cohorts IIa and IIIa, blinatumomab samples will be collected on day 1 (pre-dose, 4, 6, 8 hours after start of 9 μg/d infusion), day 2 (any time), day 8 (6-10 hours after start of 28 μg/d infusion), day 10 (any time), day 15 (6-10 hours after start of 112 μg/d infusion in cohort Ha or 56 μg/d infusion in cohort IIIa), day 19 (1 hour after pembrolizumab infusion has ended), day 26 (any time), and day 40 (any time) in cycle 1.

Pembrolizumab

Pharmacokinetic assessments will be required for all subjects receiving pembrolizumab. For cohort Ia, PK samples will be collected at pre-dose (within 24 hours before infusion) before the following infusions: on first day of pembrolizumab treatment (study day 15) and at pembrolizumab cycles 2 (study day 36), 4 (study day 78), 6 (study day 120), and 8 (study day 162), then every 4 cycles. PK post-dose samples will be collected 30 minutes post infusion on the first day of pembrolizumab treatment (study day 15), then on days 2 (study day 16), 8 (study day 22), and 15 (study day 29) of pembrolizumab cycle 1, cycle 8 day 1 (study day 162), and 30 days after discontinuation of pembrolizumab.

For cohorts Ib, IIb, and IIIb PK samples will be collected at pre-dose (within 24 hours before infusion) before the following infusions: on the first day of pembrolizumab treatment (study day 1) and at pembrolizumab cycles 2 (study day 22), 4 (study day 64), 6 (study day 106), and 8 (study day 148); then every 4 cycles. PK post-dose samples will be collected 30 minutes post-infusion on the first day of pembrolizumab treatment (study day 1) then on days 2 (study day 2), 8 (study day 8), and 15 (study day 15) of pembrolizumab cycle 1, cycle 8 day 1 (study day 148) and 30 days after discontinuation of pembrolizumab.

For cohorts Ha and IIIa, PK samples will be collected at pre-dose (within 24 hours before infusion) before the following infusions: on the first day of pembrolizumab treatment (study day 19), and at pembrolizumab cycles 2 (study day 40), 4 (study day 82), 6 (study day 124), and 8 (study day 166); then every 4 cycles.

For cohorts Ha and IIIa, pembrolizumab PK post-dose samples will be collected 30 minutes post-infusion on the first day of pembrolizumab treatment (study day 19), then on days 2 (study day 20), 8 (study day 26), and 15 (study day 33) of pembrolizumab cycle 1, cycle 8 day 1 (study day 166), and 30 days after discontinuation of pembrolizumab.

The pembrolizumab PK samples should be completed during the study visits as defined by the Schedule of Assessments (FIGS. 3-6).

Immunoglobulins

Immunoglobulins (IgG only) will be collected at time points outlined in the Schedule of Assessments (FIG. 3) to detect hypogammaglobulinemia or immunological changes.

Antibody Testing Procedures

Blood sample(s) will be collected at time points as outlined in the Schedule of Assessments (FIGS. 3-8) for the measurement of anti-blinatumomab and anti-pembrolizumab binding antibodies.

Samples testing positive for binding antibodies may be further characterized for quantity/titer, isotype, affinity, in vitro neutralizing activity, and presence of immune complexes. Additional blood samples may be obtained to rule out anti-drug antibodies during the study.

Subjects who test positive for binding antibodies and have clinical sequelae that are considered potentially related to an anti-blinatumomab or anti pembrolizumab antibody response may also be asked to return for additional follow-up testing.

### Biomarker Development

Immune Panel by Flow Cytometry

For subjects on blinatumomab, this assay will be used to monitor changes in lymphocytes (B-cell and T-cell populations) and leukocyte populations (leukocytes, lymphocytes, monocytes, and granulocytes) in peripheral blood. The rationale for an aggressive sample collection in the treatment period is to better understand the mechanism of action of the T cell response as well as potential drug resistance mechanisms.

The collection schedule is extensive to ensure adequate data is collected to better understand the mechanism of action of the T cell response elicited by the dual agent therapy, association with response, and adverse events. In cohorts Ia, Ib, IIb, and IIIb, samples will be collected days 1, 2, 3, 8, 10, 22, 43 and 64. In cohorts Ha and IIIa, samples will be collected days 1, 2, 3, 8, 10, 19, 40, and 64. All samples will be collected in the first (induction) cycle of blinatumomab only. Immune panel samples must be drawn after dexamethasone premedication but no more than 15 minutes before initiation of blinatumomab therapy.

Serum Cytokines

To monitor activation of immune effector cells, blood samples for measurement of peripheral blood cytokine levels will be taken as per the Schedule of Assessments. In cohorts Ia, Ib, IIb, and IIIb, blood samples will be collected at days 1, 2, 3, 8, 15, and 22 based on the previous phase 2 blinatumomab experience. In cohorts Ha and IIIa, blood samples will be collected at days 1, 2, 3, 8, 15, and 19. All samples will be collected in the first (induction) cycle of blinatumomab only. Cytokine samples must be drawn after dexamethasone premedication but no more than 15 minutes before initiation of blinatumomab therapy. Blood samples for cytokine measurement are also to be collected in cases of grade≥3 neurological events or CRS.

MRD by NGS (Next Generation Sequencing)

The presence or absence of MRD is becoming an increasingly important measure in hematologic malignancies and has been a key measure of the depth and quality of the treatment response in other blinatumomab studies. While MRD measurements in DLBCL is a relatively nascent field, studies have suggested inferior outcomes in subjects who have detectable MRD compared to those without detectable disease following treatment (Roschewski et al, 2015). Blood and tumor tissue samples will be collected at screening and blood samples at week 10 or time of the first disease response assessment (if done prior to week 10) and MRD will be assessed by NGS.

Pharmacogenetic Studies

If the subject consents to the optional pharmacogenetic portion of this study, PAXgene analysis may be performed. This optional pharmacogenetics analyses focus on inherited genetic variations to evaluate their possible correlation to the disease and/or responsiveness to the therapies used in this study. The goals of the optional studies include the use of genetic markers to help in the investigation of cancer and/or to identify subjects who may have positive or negative responses to blinatumomab and/or pembrolizumab. For subjects who consent to this analysis, DNA may be analyzed.

### Secondary Endpoints

The following secondary endpoints will be calculated:

ORR (including CR and PR) by Cheson Criteria;

CR rate by Cheson Criteria;

PFS will be calculated as the time from the date of first dose of blinatumomab until the date of diagnosis of progression of lymphoma per central review, or date of death, whichever is earliest. Subjects who are alive and did not have progression will be censored at the last date of tumor assessment. Progression-free survival for subjects who were enrolled in dose cohorts that were not selected for the extension part will not be calculated;

OS will be calculated as the time from the date of first dose of blinatumomab until death due to any cause. Subjects who are alive at the date that triggers the analysis will be censored at the date last known to be alive. If the date last known to be alive is after the date that triggers the analysis, the subject will be censored at the analysis trigger date;

DOR by ORR, CR, and PR will be calculated only for subjects who achieve an ORR, CR or PR. The duration will be calculated from the date a response, CR or PR, is first achieved until the earliest date of a disease assessment indicating a relapse event or death, whichever occurs first. Subjects who do not have a relapse event will be censored on their last disease assessment date. If the last disease assessment date is after the date that triggers the analysis, the subject will be censored at the analysis trigger date. A sensitivity analysis will censor subjects who receive an alloHSCT at the time of alloHSCT unless there is no assessment after the alloHSCT, in which case the last assessment prior to the alloHSCT will be used as the censoring time;

Blinatumomab PK parameters will be determined; and

Pembrolizumab PK parameters will be determined.

### REFERENCES

A clinical evaluation of the International Lymphoma Study Group classification of non-Hodgkin's lymphoma. The Non-Hodgkin's Lymphoma Classification Project. Blood. 1997 Jun. 1; 89(11):3909-3918.
Advani A, Coiffier B, Czuczman M S, et al. Safety, pharmacokinetics, and preliminary clinical activity of inotuzumab ozogamicin, a novel immunoconjugate for the treatment of B-cell non-Hodgkin's lymphoma: results of a phase I study. J Clin Oncol. 2010; 28(12):2085-2093.
Andorsky D J, Yamada R E, Said J, et al. Programmed death ligand 1 is expressed by non-hodgkin lymphomas and inhibits the activity of tumor-associated T cells. Clin Cancer Res. 2011; 17(13): 4232-4244.
A predictive model for aggressive non-Hodgkin's lymphoma. The International Non-Hodgkin's Lymphoma Prognosis Factors Project. N Eng J Med. 1993; 329(14):987 994.
Armand P, Nagler A, Weller E A, et al. Disabling immune tolerance by programmed death-1 blockade with pidilizumab after autologous hematopoietic stem-cell transplantation for diffuse large B-cell lymphoma: results of an international phase II trial. J Clin Oncol. 2013; 31(33):4199-4206.
Barosi G, Carella A, Lazzarino M, et al. Management of nodal indolent (non-marginal zone) non-Hodgkin's lymphomas: practice guidelines from the Italian Society of Hematology, Italian Society of Experimental Hematology and Italian Group for Bone Marrow Transplantation. Haematologica. 2005; 90(9): 1236-1257.
Bellati F, Visconti V, Napoletano C, et al. Immunology of gynecologic neoplasms: analysis of the prognostic significance of the immune status. Curr Cancer Drug Targets. 2009; 9(4):541-565.
Blank C, Brown I, Peterson A C, et al. PD-L1/B7H-1 inhibits the effector phase of tumor rejection by T cell receptor (TCR) transgenic CD8+ T cells. Cancer Res. 2004; 64(3): 1140-1145.
Amgen Blinatumomab Investigator's Brochure. Thousand Oaks, Calif. Amgen Inc.
Bremnes R M, Al-Shibli K, Donnem T, et al. The role of tumor-infiltrating immune cells and chronic inflammation at the tumor site on cancer development, progression, and prognosis: emphasis on non-small cell lung cancer. J Thorac Oncol. 2011; 6(4):824-833.
Brookmeyer R and Crowly J. A confidence interval for the median survival time. Biometrics. 1982; 29-41.
Chang W J, Du Y, Zhao X, Ma L Y, Cao G W. Inflammation-related factors predicting prognosis of gastric cancer. World J Gastroenterol. 2014; 20(16):4586-4596.
Cheng X, Veverka V, Radhakrishnan A, et al. Structure and interactions of the human programmed cell death 1 receptor. J Biol Chem, 2013; 288(17):11771-11785.
Cheson B D, Fisher R I, Barrington S F, et al. Recommendations for initial evaluation, staging, and response assessment of Hodgkin and non-Hodgkin lymphoma: the Lugano classification. J Clin Oncol. 2014; 32(27):3059-3068.
Cheson B D, Pfistner B, Juweid M E, et al. Revised response criteria for malignant lymphoma. J Clin Oncol. 2007; 25(5):579-586.
Clopper C J and Pearson E G. The use of confidence or fiducial limits illustrated in the case of the binomial. Biometrica 1934; 26(4):404-413.
Curran M A, Montalvo W, Yagita H, Allison J P. PD-1 and CTLA-4 combination blockade expands infiltrating T cells and reduces regulatory T and myeloid cells within B16 melanoma tumors. Proc Natl Acad Sci USA. 2010; 107(9):4275-4280.
Dang, Nam H, et al. Randomized, phase 3 trial of inotuzumab ozogamicin plus rituximab (R-InO) versus chemotherapy for relapsed/refractory aggressive B-cell non-Hodgkin lymphoma (B-NHL). ASCO Annual Meeting Proceedings. 2014; 32(15).
Disis M L. Immune regulation of cancer. J Clin Oncol. 2010; 28(29):4531-4538.
Dreier T, Lorenczewski G, Brandl C, et al. Extremely potent, rapid and costimulation independent cytotoxic T-cell response against lymphoma cells catalyzed by a single chain bispecific antibody. Int J Cancer. 2002; 100(6):690-697.
Dunn G P, Dunn I F, Curry W T. Focus on TILs: Prognostic significance of tumor infiltrating lymphocytes in human glioma. Cancer Immun, 2007; 7:12.
EuroQol Group. EuroQol—a new facility for the measurement of health-related quality of life. Health Policy. 1990; 16(3):199-208.
Feuchtinger T, Feucht J, Kayser S, et al. Leukemia Related Co-Stimulation/Co Inhibition Predict T Cell Attack of Acute Lymphoblastic Leukemia Mediated by Blinatumomab. Blood, 2015; 126:3764.
Fiona K and Ruth P. Radioimmunotherapy (RIT) in non-Hodgkin lymphoma. Targeted Oncology, 2007; 2(3): 173-179.
Fisher S G, Fisher R I. The epidemiology of non-Hodgkin's lymphoma. Oncogene. 2004; 23(38):6524-6534.
Friedberg W. Relapsed/refractory diffuse large B-cell lymphoma. ASH Education Program Book 2011; 2011: 498-505.
Gabellier L and Cartron G. Obinutuzumab for relapsed or refractory indolent non Hodgkin's lymphomas. Ther ad Hematol. 2016; 7(2):85-93.
Gisselbrecht C, Glass B, Mounier N, et al. Salvage regimens with autologous transplantation for relapsed large B-cell lymphoma in the rituximab era. J Clin Oncol, 2010; 28(27):4184-4190.
Goebeler M, et al. Blinatumomab (CD3/CD19 Bite (R) Antibody) results in a high response rate in patients with relapsed non-hodgkin lymphoma (NHL) including mantle cell lymphoma (MCL) and diffuse large B cell lymphoma (DLBCL). in ONKOLOGIE. 2011. KARGER ALLSCHWILERSTRASSE 10, CH-4009 BASEL, SWITZERLAND
Gooden M J, de Bock G H, Leffers N, Daemen T, Nijman H W. The prognostic influence of tumour-infiltrating lymphocytes in cancer: a systematic review with meta-analysis. Br J Cancer, 2011; 105(1):93-103.
Goy A, Younes A, McLaughlin P, et al. Phase II study of proteasome inhibitor bortezomib in relapsed or refractory B-cell non-Hodgkin's lymphoma. J Clin Oncol. 2005; 23(4):667-675.
Hans C P, Weisenburger D D, Greiner T C, et al. Confirmation of the molecular classification of diffuse large B-cell lymphoma by immunohistochemistry using a tissue microarray. Blood. 2004. 103(1):275-282.
Hartge P and Wang S S. Overview of the etiology and epidemiology of lymphoma. Non Hodgkin's Lymphomas. Lippincott, Philadelphia, 2004.711-727.
Hirano F, Kaneko K, Tamura H, et al., Blockade of B7-H1 and PD-1 by monoclonal antibodies potentiates cancer therapeutic immunity. Cancer Res. 2005; 65(3): 1089-1096.
Hlubocky F J, Webster K, Beaumont J, Cashy J, Paul D, Abernethy A, Syrjala K L, Von Roenn J, Cella D. A preliminary study of a health related quality of life assessment of priority symptoms in advanced lymphoma: the National Comprehensive Cancer Network-Function Assessment of Cancer Therapy—Lymphoma Symptom Index. Leuk Lymphoma. 2013; 43(9): 1942-1946.
Huang X, Venet F, Wang Y L, et al. PD-1 expression by macrophages plays a pathologic role in altering microbial clearance and the innate inflammatory response to sepsis. Proc Natl Acad Sci USA. 2009; 106(15):6303-6308.
Kalbfleisch J D and Prentice R L. The Statistical Analysis of Failure Time Data. John Wiley, New York Rev, 1980; 4(2):25.
Karim R, Jordanova E S, Piersma S J, et al. Tumor-expressed B7-H1 and B7-DC in relation to PD-1+ T-cell infiltration and survival of patients with cervical carcinoma. Clin Cancer Res. 2009; 15(20):6341-6347.
Keir M E, Butte M J, Freeman G J, Sharpe A H. PD-1 and its ligands in tolerance and immunity. Annu Rev Immunol. 2008; 26:677-704.
Kenderian S S, Ruella M, Shestova O, et al. Identification of PD1 and TIM3 As Checkpoints That Limit Chimeric Antigen Receptor T Cell Efficacy in Leukemia (Abstract). Biol Bone Mar Transpl. 2016; 22: S19-S481.[abstract]
Kiyasu J, Miyoshi H, Hirata A, et al. Expression of programmed cell death ligand 1 is associated with poor overall survival in patients with diffuse large B cell lymphoma. Blood. 2015; 126(19):2193-2201.
Kim S T, Jeong H, Woo O H, et al. Tumor-infiltrating lymphocytes, tumor characteristics, and recurrence in patients with early breast cancer. Am J Clin Oncol. 2013; 36(3):224 231.
Kirk R. Risk factors. CD8+:FOXP3+ cell ratio is a novel survival marker for colorectal cancer. Nat Rev Clin Oncol. 2010; 7(6):299.
Köhnke T, Krupka C, Tischer J, Knsel T, Subklewe M. Increase of PD-L1 expressing B precursor ALL cells in a patient resistant to the CD19/CD3-bispecific T cell engager antibody blinatumomab. J Hematol Oncol. 2015; 8(1):111.
Krupka C, Kufer P, Kischel R, et al., Blockade of the PD-1/PD-L1 axis augments lysis of AML cells by the CD33/CD3 BiTE antibody construct AMG 330: reversing a T cell induced immune escape mechanism. Leukemia. 2016; 30(2):484-491.
Lázár-Molnár E, Yan Q, Cao E, Ramagopal U, Nathenson S G, Almo S C. Crystal structure of the complex between programmed death-1 (PD-1) and its ligand PD-L2. Proc Natl Acad Sci USA. 2008; 105(30):10483-10488.
Lazlo G S, Gudgeon C J, Harrington K H, Walter R B. T-cell ligands modulate the cytolytic activity of the CD33/CD3 BiTE antibody construct, AMG 330 [abstract]. Bl Cancer J. 2015; 5: e340.
Lenz G, Staudt L M. Aggressive lymphomas. N Engl J Med. 2010; 362(15): 1417-1429.
Lenz G, Wright G W, Emre N C, et al. Molecular subtypes of diffuse large B-cell lymphoma arise by distinct genetic pathways. Proc Natl Acad Sci USA, 2008; 105(36): 13520-13525.
Lesokhin A M, Ansell S M, Armand P, et al. Nivolumab in Patients With Relapsed or Refractory Hematologic Malignancy: Preliminary Results of a Phase 1b Study. J Clin Oncol. 2016; 34(23):2698-2704.
Lin D Y, Tanaka Y, Iwasaki M, et al., The PD-1/P D-L1 complex resembles the antigen binding Fv domains of antibodies and T cell receptors. Proc Natl Acad Sci USA. 2008; 105(8):3011-3016.
Liu F, Lang R, Zhao J, et al. CD8(+) cytotoxic T cell and FOXP3(+) regulatory T cell infiltration in relation to breast cancer survival and molecular subtypes. Breast Cancer Res Treat. 2011; 130(2):645-655.
Martelli M, Ferreri A J, Agostinelli C, Di Rocco A, Pfreundschuh M, Pileri S A. Diffuse large B-cell lymphoma. Crit Rev Oncol Hematol. 2013; 87(2): 146-171.
Mathai A M, Kapadia M J, Alexander J, Kemochan L E, Swanson P E, Yeh M M. Role of Foxp3-positive tumor-infiltrating lymphocytes in the histologic features and clinical outcomes of hepatocellular carcinoma. Am J Surg Pathol. 2012; 36(7):980-986.
Mei Z, Liu Y, Liu C, Cui L. Tumour-infiltrating inflammation and prognosis in colorectal cancer: systematic review and meta-analysis. Br J Cancer. 2014; 110(6): 1595-1605.
Morgan G, Vomanen M, Puitinen J, et al. Changing trends in the incidence of non Hodgkin's lymphoma in Europe. Ann Oncol. 1997; 8(suppl 2):49-54.
Morschhauser F, Kraeber-Bodéré F, Wegener W A, et al. High rates of durable responses with anti-CD22 fractionated radioimmunotherapy: results of a multicenter, phase I/II study in non-Hodgkin's lymphoma. J Clin Oncol. 2010; 28(23):3709-3716.
Nishimura H, Agata Y, Kawasaki A, et al., Developmentally regulated expression of the PD-1 protein on the surface of double-negative (CD4-CD8-) thymocytes. Int Immunol. 1996; 8(5):773-780.
Nomi T, Sho M, Akahori T, et al. Clinical significance and therapeutic potential of the programmed death-1 ligand/programmed death-1 pathway in human pancreatic cancer. Clin Cancer Res. 2007; 13(7):2151-2157.
Nosho K, Baba Y, Tanaka N, et al. Tumour-infiltrating T-cell subsets, molecular changes in colorectal cancer, and prognosis: cohort study and literature review. J Pathol. 2010; 222(4):350-366.
Nyman H, Adde M, Karjalainen-Lindsberg M L, et al. Prognostic impact of immunohistochemically defined germinal center phenotype in diffuse large B cell lymphoma patients treated with immunochemotherapy. Blood. 2007; 109(11):4930 4935.
Oble D A, Loewe R, Yu P, Mihm M C Jr. Focus on TILs: prognostic significance of tumor infiltrating lymphocytes in human melanoma. Cancer Immun. 2009; 9:3.
Oken M M, Creech R H, Tormey D C, Horton J, Davis T E, McFadden E T, Carbone P P. Toxicity and response criteria of the Eastern Cooperative Oncology Group. Am J Clin Oncol. 1982; 5(6):649-655.
Osada T, Patel S P, Hammond S A, et al. CEA/CD3-bispecific T cell-engaging (BiTE) antibody-mediated T lymphocyte cytotoxicity maximized by inhibition of both PD1 and PD L1. Cancer Immun Immunother. 2015; 64:677-688.
Ott P A, Hodi F S, Robert C. CTLA-4 and PD-1/PD-L1 blockade: new immunotherapeutic modalities with durable clinical benefit in melanoma patients. Clin Cancer Res. 2013; 19(19):5300-5309.
Pedoeem A, Azoulay-Alfaguter I, Strazza M, Silverman G J, Mor A. Programmed death-1 pathway in cancer and autoimmunity. Clin Immunol. 2014; 153(1):145-152.
Pefia-Cruz V, McDonough S M, Diaz-Griffero F, Crum C P, Carrasco R D, Freeman G J. PD-1 on immature and PD-1 ligands on migratory human Langerhans cells regulate antigen-presenting cell activity. J Invest Dermatol. 2010; 130(9):2222-2230.
Pettengell R, Coiffier B, Narayanan G, et al. Pixantrone dimaleate versus other chemotherapeutic agents as a single-agent salvage treatment in patients with relapsed or refractory aggressive non-Hodgkin lymphoma: a phase 3, multicentre, open-label, randomised trial. Lancet Oncol. 2012; 13(7):696-706.
Pilon-Thomas S, Mackay A, Vohra N, Mulé J J. Blockade of programmed death ligand 1 enhances the therapeutic efficacy of combination immunotherapy against melanoma. J Immunol. 2010; 184(7):3442-3449.
Preston C C, Maurer M J, Oberg A L, et al. The ratios of CD8+ T cells to CD4+CD25+ FOXP3+ and FOXP3− T cells correlate with poor clinical outcome in human serous ovarian cancer. PLoS One. 2013; 8(11):e80063.
Roschewski M, Dunleavy K, Pittaluga S, et al. Circulating tumor DNA and CT monitoring in patients with untreated diffuse large B-cell lymphoma: a correlative biomarker study. Lancet Oncol, 2015; 16(5):541-540.
Rosenwald A, Wright G, Chan W C, et al. The use of molecular profiling to predict survival after chemotherapy for diffuse large B-cell lymphoma. N Engl J Med. 2002; 346(25):1937 1947.
Salgado R, Denkert C, Demaria S, et al. Harmonization of the evaluation of tumor infiltrating lymphocytes (TILs) in breast cancer: recommendations by an international TILs-working group 2014. Ann Oncol. 2015; 26(2):259-271.
Salles G, Morschhauser F, Lamy T, et al. Phase 1 study results of the type II glycoengineered humanized anti-CD20 monoclonal antibody obinutuzumab (GA101) in B-cell lymphoma patients. Blood. 2012; 119(22):5126-5132.
Sanmamed M F, Chen L. Inducible expression of B7-H1 (PD-L1) and its selective role in tumor site immune modulation. Cancer J. 2014; 20(4):256-261.
Schatton T, Scolyer R A, Thompson J F, Mihm M C Jr. Tumor-infiltrating lymphocytes and their significance in melanoma prognosis. Methods Mol Biol. 2014; 1102:287-324.
Schlereth B, Kleindienst P, Fichtner I, et al. Potent inhibition of local and disseminated tumor growth in immunocompetent mouse models by a by specific antibody construct specific for Murine CD3. Cancer Immunol Immunother. 2006; 55(7):785-796.
Schreiber R D, Old L J, Smyth M J. Cancer immunoediting: integrating immunity's roles in cancer suppression and promotion. Science. 2011; 331(6024): 1565-1570.
Sehn L H, Berry B, Chhanabhai M, et al. The revised International Prognostic Index (R IPI) is a better predictor of outcome than the standard IPI for patients with diffuse large B-cell lymphoma treated with R-CHOP. Blood. 2007; 109(5):1857-1861.
Sheppard K A, Fitz L J, Lee J M, et al., PD-1 inhibits T-cell receptor induced phosphorylation of the ZAP70/CD3zeta signalosome and downstream signaling to PKCtheta. FEBS Lett. 2004; 574(1-3):37-41.
Shirabe K, Motomura T, Muto J, et al., Tumor-infiltrating lymphocytes and hepatocellular carcinoma: pathology and clinical management. Int J Clin Oncol. 2010; 15(6):552-558.
Smith S M, van Besien K, Karrison T, et al. Temsirolimus has activity in non-mantle cell Non-Hodgkin's lymphoma subtypes: The University of Chicago Phase II consortium. J Clin Oncol. 2010; 28(31):4740-4746.
Spranger S, Koblish H K, Horton B, Scherle P A, Newton R, Gajewski T F. Mechanism of tumor rejection with doublets of CTLA-4, PD-1/PD-L1, or IDO blockade involves restored IL-2 production and proliferation of CD8(+) T cells directly within the tumor microenvironment. J Immunother Cancer. 2014; 2:3.
Stopeck A T, Unger J M, Rimsza L M, et al. A phase II trial of single agent bevacizumab in patients with relapsed, aggressive non-Hodgkin lymphoma: Southwest oncology group study S0108. Leuk Lymphoma. 2009; 50(5):728-735.
Strome S E, Dong H, Tamura H, et al. B7-H1 blockade augments adoptive T-cell immunotherapy for squamous cell carcinoma. Cancer Res. 2003; 63(19):6501-6505.
Swerdlow S H, Campo E, Pileri S A, et al. The 2016 Revision of the World Health Organization (WHO) Classification of lymphoid neoplasms. Blood, 2016; 127(20):2375 2390.
Talmadge J E. Immune cell infiltration of primary and metastatic lesions: mechanisms and clinical impact. Semin Cancer Biol. 2011; 21(2): 131-138.
Taube J M, Anders R A, Young G D, et al., Colocalization of inflammatory response with B7-h 1 expression in human melanocytic lesions supports an adaptive resistance mechanism of immune escape. Sci Transl Med. 2012; 4(127): 127ra37.
Tilly H, Vitolo U, Walewski J, et al. Diffuse large B-cell lymphoma (DLBCL): ESMO Clinical Practice Guidelines for diagnosis, treatment, and follow-up. Annals of Oncology, 2015; 26(supp5): 116-125.
Topalian S L, Drake C G, Pardoll D M. Targeting the PD-1/B7-H1(PD-L1) pathway to activate anti-tumor immunity. Curr Opin Immunol. 2012; 24(2):207-212.
Tmeny M, Verhoef G, Dyer M J S, et al. “Starlyte phase II study of coltuximab ravtansine (CoR, SAR3419) single agent: Clinical activity and safety in patients (pts) with relapsed/refractory (R/R) diffuse large B-cell lymphoma (DLBCL; NCT01472887).” ASCO Annual Meeting Proceedings. Vol. 32. No. 15_suppl. 2014.
Uppaluri R, Dunn G P, Lewis J S Jr. Focus on TILs: prognostic significance of tumor infiltrating lymphocytes in head and neck cancers. Cancer Immun. 2008; 8:16.
Vacirca J L, Acs P I, Shimkus B J, et al. Bendamustine/rituximab in relapsed or refractory diffuse large B-cell lymphoma. ASCO Annual Meeting Proceedings. Vol. 28. No. 15_suppl. 2010.
Viardot A, Goebeler M, Hess G, et al. Treatment of relapsed/refractory diffuse large B cell lymphoma with the bispecific T-cell engager (BiTE®) antibody construct blinatumomab: primary analysis results from an open-label, phase 2 study. Blood. 2014; 124(21):4460.
Weber J. Immune checkpoint proteins: a new therapeutic paradigm for cancer—preclinical background: CTLA-4 and PD-1 blockade. Semin Oncol. 2010; 37(5):430 439.
Witzig T E, Vose J M, Zinzani P L, et al. An international phase II trial of single-agent lenalidomide for relapsed or refractory aggressive B-cell non-Hodgkin's lymphoma. Ann Oncol. 2011; 22(7): 1622-1627.
Yao S and Chen L. PD-1 as an immune modulatory receptor. Cancer J. 2014; 20(4):262 264.
Yoon H H, Orrock J M, Foster N R, Sargent D J, Smyrk T C, Sinicrope F A. Prognostic impact of FoxP3+ regulatory T cells in relation to CD8+ T lymphocyte density in human colon carcinomas. PLoS One. 2012; 7(8):e42274.
Zelenetz A D, Gordon L I, Wierda W G, et al. Diffuse Large B-Cell Lymphoma Version 1.2016. J Natl Compr Canc Netw, 2016; 14(2): 196-231.
Ziepert M, Hasenclever D, Kuhnt E, et al. Standard International prognostic index remains a valid predictor of outcome for patients with aggressive CD20+B-cell lymphoma in the rituximab era. J Clin Oncol. 2010; 28(14):2373-2380.
Zimmerman Z, Maniar T, Nagorsen D. Unleashing the clinical power of T cells: CD19/CD3 bi-specific T cell engager (BiTE(R)) antibody construct blinatumomab as a potential therapy. Int Immunol. 2015; 27(1):31-37.
Zhang L, Gajewski T F, Kline J. PD-1/PD-L1 interactions inhibit antitumor immune responses in a murine acute myeloid leukemia model. Blood. 2009; 114(8): 1545-1552.
Zhang X, Schwartz J C, Guo X, et al., Structural and functional analysis of the costimulatory receptor programmed death-1. Immunity. 2004; 20(3):337-347.

What is claimed is:

1. A method of treating diffuse large B cell lymphoma (DLBCL) in a subject comprising:
administering blinatumomab or a blinatumomab variant to the subject; and
administering pembrolizumab, a pembrolizumab variant or an antigen-binding fragment thereof to the subject, thereby treating DLBCL in the subject.

2. The method of claim 1, wherein the DLBCL is refractory to previous therapy or is relapsed after previous therapy.

3. The method of claim 1, wherein the blinatumomab or the blinatumomab variant is administered to the subject systemically and/or the pembrolizumab, the pembrolizumab variant or the antigen-binding fragment thereof is administered to the subject systemically.

4. The method of claim 1, wherein a first dose of the blinatumomab or the blinatumomab variant is administered to the subject prior to the administration of a first dose of the pembrolizumab, the pembrolizumab variant or the antigen-binding fragment thereof.

5. The method of claim 1, wherein a first dose of the blinatumomab or the blinatumomab variant is administered to the subject concomitant with the administration of a first dose of the pembrolizumab, the pembrolizumab variant or the antigen-binding fragment thereof.

6. The method of claim 4, wherein the blinatumomab or the blinatumomab variant is administered daily.

7. The method of claim 4, wherein a secondary dose of pembrolizumab, pembrolizumab variant or antigen-binding fragment thereof is administered approximately 21 days after the first dose of the pembrolizumab, pembrolizumab variant or antigen-binding fragment thereof.

8. The method of claim 7, wherein one or more additional secondary doses of pembrolizumab, pembrolizumab variant or antigen-binding fragment thereof are administered approximately every 21 days.

9. The method of claim 4, wherein the pembrolizumab, the pembrolizumab variant or the antigen-binding fragment thereof is administered at a dose of about 200 mg.

10. The method of claim 4, wherein the blinatumomab or the blinatumomab variant is administered at an initial dose of at least about 9 μg/d.

11. The method of claim 10, wherein the blinatumomab or the blinatumomab variant is administered at a maintenance dose of about 28 μg/d, about 56 μg/d or about 112/d μg.

12. The method of claim 6, wherein the blinatumomab or the blinatumomab variant is administered in a first treatment cycle, followed by a treatment-free cycle, followed by one or more consolidation cycles.

13. The method of claim 12, wherein the first treatment cycle is between about 49 and about 63 days.

14. The method of claim 13, wherein the first treatment cycle is about 56 days.

15. The method of claim 12, wherein the treatment-free cycle is between about 14 and about 28 days.

16. The method of claim 15, wherein the treatment-free cycle is about 21 days.

17. The method of claim 12, wherein each of the one or more consolidation cycles are between about 14 and about 28 days.

18. The method of claim 17, wherein each of the one or more consolidation cycles are about 21 days.

19. The method of claim 4, wherein the first dose of the blinatumomab or the blinatumomab variant is administered to the subject on day 1 and the first dose of the pembrolizumab, the pembrolizumab variant or the antigen-binding fragment thereof is administered to the subject on day 1.

20. The method of claim 4, wherein the first dose of the blinatumomab or the blinatumomab variant is administered to the subject on day 1 and the first dose of the pembrolizumab, the pembrolizumab variant or the antigen-binding fragment thereof is administered to the subject on about day 15.

21. The method of claim 4, wherein the first dose of the blinatumomab or the blinatumomab variant is administered to the subject on day 1 and the first dose of the pembrolizumab, the pembrolizumab variant or the antigen-binding fragment thereof is administered to the subject on about day 19.

22. The method of claim 3, wherein the blinatumomab or the blinatumomab variant is administered by continuous intravenous infusion (CIVI).

23. The method of claim 3, wherein the pembrolizumab, the pembrolizumab variant or the antigen-binding fragment thereof is administered by intravenous (IV) infusion.

24. A method of treating DLBCL in a subject comprising:
administering a dose of about 9 μg blinatumomab or a blinatumomab variant to the subject on each of treatment days 1 to 7; and
administering an initial dose of about 200 mg pembrolizumab, a pembrolizumab variant or an antigen-binding fragment thereof to the subject on treatment day 1, and one or more subsequent doses of about 200 mg pembrolizumab, a pembrolizumab variant or an antigen-binding fragment thereof approximately every 21 days.

25. The method of claim 24, further comprising administering a dose of about 28 μg blinatumomab or a blinatumomab variant to the subject on each of treatment days 8 to 14.

26. The method of claim 25, further comprising administering a dose of about 112 μg blinatumomab or a blinatumomab variant to the subject on each of treatment days 22 to 56.

27. The method of claim 25, further comprising administering a dose of about 56 μg blinatumomab or a blinatumomab variant to the subject on each of treatment days 15 to 56.

28. The method of claim 24, further comprising administering a dose of about 28 μg blinatumomab or a blinatumomab variant to the subject on each of treatment days 8 to 56.

29. A method of treating DLBCL in a subject comprising:
administering a dose of about 9 μg blinatumomab or a blinatumomab variant to the subject on each of days 1 to 7 of a first treatment cycle; and
administering an initial dose of about 200 mg pembrolizumab, a pembrolizumab variant or an antigen-binding fragment thereof to the subject on day 15 of the first treatment cycle, and one or more subsequent doses of about 200 mg pembrolizumab, a pembrolizumab variant or an antigen-binding fragment thereof approximately every 21 days.

30. The method of claim 29, further comprising administering a dose of about 28 μg blinatumomab or a blinatumomab variant to the subject on each of treatment days 8 to 56.

31. A method of treating DLBCL in a subject comprising:
administering a dose of about 9 μg blinatumomab or a blinatumomab variant to the subject on each of days 1 to 7 of a first treatment cycle; and
administering an initial dose of about 200 mg pembrolizumab, a pembrolizumab variant or an antigen-binding fragment thereof to the subject on day 19 of the first treatment cycle, and one or more subsequent doses of about 200 mg pembrolizumab, a pembrolizumab variant or an antigen-binding fragment thereof approximately every 21 days.

32. The method of claim 31, further comprising administering a dose of about 28 μg blinatumomab or a blinatumomab variant to the subject on each of days 8 to 14 of the first treatment cycle.

33. The method of claim 32, further comprising administering a dose of about 112 μg blinatumomab or a blinatumomab variant to the subject on each of days 22 to 56 of the first treatment cycle.

34. The method of claim 32, further comprising administering a dose of about 56 μg blinatumomab or a blinatumomab variant to the subject on each of days 15 to 56 of the first treatment cycle.

35. The method of claim 31, further comprising administering a dose of about 28 μg blinatumomab or a blinatumomab variant to the subject on each of days 8 to 56 of the first treatment cycle.

36. The method of any of claims 24, 29 or 31, further comprising a treatment-free cycle in which blinatumomab or a blinatumomab variant is not administered to the subject for between about 14 and about 28 days.

37. The method of claim 36, wherein the treatment-free cycle is about 21 days.

38. The method of claim 36, further comprising one or more consolidated cycles wherein about 29 μg, about 56 μg or about 112 μg of blinatumomab or a blinatumomab variant is administered to the subject daily for between about 14 and about 28 days.

39. The method of claim 38, wherein the one or more consolidated cycles are each about 21 days.

40. A method of treating DLBCL in a subject comprising:
administering a dose of about 9 μg blinatumomab or a blinatumomab variant to the subject on each of days 1 to 7 of a first treatment cycle, and a dose of about 28 μg blinatumomab or a blinatumomab variant to the subject on each of days 8 to 56 of the first treatment cycle; and
administering an initial dose of about 200 mg pembrolizumab, a pembrolizumab variant or an antigen-binding fragment thereof to the subject on treatment day 1, and one or more subsequent doses of about 200 mg pembrolizumab, a pembrolizumab variant or an antigen-binding fragment thereof approximately every 21 days.

41. A method of treating DLBCL in a subject comprising:
administering a dose of about 9 μg blinatumomab or a blinatumomab variant to the subject on each of days 1 to 7 of a first treatment cycle, a dose of about 28 μg blinatumomab or a blinatumomab variant to the subject on each of days 8 to 14 of the first treatment cycle, and a dose of about 112 μg blinatumomab or a blinatumomab variant to the subject on each of days 15 to 56 of the first treatment cycle; and
administering an initial dose of about 200 mg pembrolizumab, a pembrolizumab variant or an antigen-binding fragment thereof to the subject on day 1 of the first treatment cycle, and one or more subsequent doses of about 200 mg pembrolizumab, a pembrolizumab variant or an antigen-binding fragment thereof approximately every 21 days.

42. A method of treating DLBCL in a subject comprising:
administering a dose of about 9 μg blinatumomab or a blinatumomab variant to the subject on each of days 1 to 7 of a first treatment cycle, a dose of about 28 μg blinatumomab or a blinatumomab variant to the subject on each of days 8 to 14 of the first treatment cycle, and a dose of about 56 lag blinatumomab or a blinatumomab variant to the subject on each of days 15 to 56 of the first treatment cycle; and
administering an initial dose of about 200 mg pembrolizumab, a pembrolizumab variant or an antigen-binding fragment thereof to the subject on day 1 of the first treatment cycle, and one or more subsequent doses of about 200 mg pembrolizumab, a pembrolizumab variant or an antigen-binding fragment thereof approximately every 21 days.

43. A method of treating DLBCL in a subject comprising:
administering a dose of about 9 μg blinatumomab or a blinatumomab variant to the subject on each of days 1 to 7 of a first treatment cycle, and a dose of about 28 μg blinatumomab or a blinatumomab variant to the subject on each of days 8 to 56 of the first treatment cycle; and
administering an initial dose of about 200 mg pembrolizumab, a pembrolizumab variant or an antigen-binding fragment thereof to the subject on day 15 of the first treatment cycle, and one or more subsequent doses of about 200 mg pembrolizumab, a pembrolizumab variant or an antigen-binding fragment thereof approximately every 21 days.

44. A method of treating DLBCL in a subject comprising:
administering a dose of about 9 μg blinatumomab or a blinatumomab variant to the subject on each of days 1 to 7 of the first treatment cycle, a dose of about 28 μg blinatumomab or a blinatumomab variant to the subject on each of days 8 to 14 of the first treatment cycle, and a dose of about 112 μg blinatumomab or a blinatumomab variant to the subject on each of days 15 to 56 of the first treatment cycle;
and
administering an initial dose of about 200 mg pembrolizumab, a pembrolizumab variant or an antigen-binding fragment thereof to the subject on day 19 of the first treatment cycle, and one or more subsequent doses of about 200 mg pembrolizumab, a pembrolizumab variant or an antigen-binding fragment thereof approximately every 21 days.

45. A method of treating DLBCL in a subject comprising:
administering a dose of about 9 μg blinatumomab or a blinatumomab variant to the subject on each of days 1 to 7 of the first treatment cycle, a dose of about 28 μg blinatumomab or a blinatumomab variant to the subject on each of days 8 to 14 of the first treatment cycle, and a dose of about 56 μg blinatumomab or a blinatumomab variant to the subject on each of days 15 to 56 of the first treatment cycle:
and
administering an initial dose of about 200 mg pembrolizumab, a pembrolizumab variant or an antigen-binding fragment thereof to the subject on day 19 of the first treatment cycle, and one or more subsequent doses of about 200 mg pembrolizumab, a pembrolizumab variant or an antigen-binding fragment thereof approximately every 21 days.

46. A method of treating DLBCL in a subject comprising:
administering a dose of about 28 μg, about 56 μg, or about 112 μg blinatumomab or a blinatumomab variant to the subject daily starting at treatment day 1; and
administering an initial dose of about 200 mg pembrolizumab, a pembrolizumab variant or an antigen-binding fragment thereof approximately every 21 days starting at treatment day 1.

47. A method of treating DLBCL in a subject comprising:
administering a dose of about 28 μg, about 56 μg, or about 112 μg blinatumomab or a blinatumomab variant to the subject daily starting at treatment day 1; and administering an initial dose of about 200 mg pembrolizumab, a pembrolizumab variant or an antigen-binding fragment thereof approximately every 21 days starting at treatment day 15.

48. A method of treating DLBCL in a subject comprising:
administering a dose of about 28 μg, about 56 μg, or about 112 μg blinatumomab or a blinatumomab variant to the subject daily starting at treatment day 1; and
administering an initial dose of about 200 mg pembrolizumab, a pembrolizumab variant or an antigen-binding fragment thereof approximately every 21 days starting at treatment day 19.

49. Blinatumomab or a blinatumomab variant for use in treating DLBCL in a subject in combination with pembrolizumab, a pembrolizumab variant or an antigen-binding fragment thereof.

50. Pembrolizumab, a pembrolizumab variant or an antigen-binding fragment thereof for use in treating DLBCL in a subject in combination with blinatumomab or a blinatumomab variant.

51. The use of claim 49 or 50, wherein the DLBCL is refractory to previous therapy or is relapsed after previous therapy.

52. The use of claim 49 or 50, wherein the blinatumomab or the blinatumomab variant is administered to the subject systemically and/or the pembrolizumab, the pembrolizumab variant or the antigen-binding fragment thereof is administered to the subject systemically.

53. The use of claim 49 or 50, wherein a first dose of the blinatumomab or the blinatumomab variant is administered to the subject prior to the administration of a first dose of the pembrolizumab, the pembrolizumab variant or the antigen-binding fragment thereof.

54. The use of claim 49 or 50, wherein a first dose of the blinatumomab or the blinatumomab variant is administered to the subject concomitant with the administration of a first dose of the pembrolizumab, the pembrolizumab variant or the antigen-binding fragment thereof.

55. The use of claim 53, wherein the blinatumomab or the blinatumomab variant is administered daily.

56. The use of claim 53, wherein a secondary dose of pembrolizumab, pembrolizumab variant or antigen-binding fragment thereof is administered approximately 21 days after the first dose of the pembrolizumab, pembrolizumab variant or antigen-binding fragment thereof.

57. The use of claim 56, wherein one or more additional secondary doses of pembrolizumab, pembrolizumab variant or antigen-binding fragment thereof are administered approximately every 21 days.

58. The use of claim 53, wherein the pembrolizumab, the pembrolizumab variant or the antigen-binding fragment thereof is administered at a dose of about 200 mg.

59. The use of claim 53, wherein the blinatumomab or the blinatumomab variant is administered at an initial dose of at least about 9 μg/d.

60. The use of claim 59, wherein the blinatumomab or the blinatumomab variant is administered at a maintenance dose of about 28 μg/d, about 56 μg/d or about 112/μg.

61. The use of claim 55, wherein the blinatumomab or the blinatumomab variant is administered in a first treatment cycle, followed by a treatment-free cycle, followed by one or more consolidation cycles.

62. The use of claim 61, wherein the first treatment cycle is between about 49 and about 63 days.

63. The use of claim 62, wherein the first treatment cycle is about 56 days.

64. The use of claim 61, wherein the treatment-free cycle is between about 14 and about 28 days.

65. The use of claim 64, wherein the treatment-free cycle is about 21 days.

66. The use of claim 65, wherein each of the one or more consolidation cycles are between about 14 and about 28 days.

67. The use of claim 66, wherein each of the one or more consolidation cycles are about 21 days.

68. The use of claim 53, wherein the first dose of the blinatumomab or the blinatumomab variant is administered to the subject on day 1 and the first dose of the pembrolizumab, the pembrolizumab variant or the antigen-binding fragment thereof is administered to the subject on day 1.

69. The use of claim 53, wherein the first dose of the blinatumomab or the blinatumomab variant is administered to the subject on day 1 and the first dose of the pembrolizumab, the pembrolizumab variant or the antigen-binding fragment thereof is administered to the subject on about day 15.

70. The use of claim 53, wherein the first dose of the blinatumomab or the blinatumomab variant is administered to the subject on day 1 and the first dose of the pembrolizumab, the pembrolizumab variant or the antigen-binding fragment thereof is administered to the subject on about day 19.

71. The use of claim 52, wherein the blinatumomab or the blinatumomab variant is administered by CIVI.

72. The use of claim 52, wherein the pembrolizumab, the pembrolizumab variant or the antigen-binding fragment thereof is administered by IV infusion.

73. A medicament comprising blinatumomab or a blinatumomab variant for use in treating DLBCL in a subject in combination with pembrolizumab, a pembrolizumab variant or an antigen-binding fragment thereof.

74. A medicament comprising pembrolizumab, a pembrolizumab variant or an antigen-binding fragment thereof for use in treating DLBCL in a subject in combination with blinatumomab or a blinatumomab variant.

75. The medicament of claim 73 or 74, wherein the DLBCL is refractory to previous therapy or is relapsed after previous therapy.

76. The medicament of claim 73 or 74, wherein the blinatumomab or the blinatumomab variant is administered to the subject systemically and/or the pembrolizumab, the pembrolizumab variant or the antigen-binding fragment thereof is administered to the subject systemically.

77. The medicament of claim 73 or 74, wherein a first dose of the blinatumomab or the blinatumomab variant is administered to the subject prior to the administration of a first dose of the pembrolizumab, the pembrolizumab variant or the antigen-binding fragment thereof.

78. The medicament of claim 73 or 74, wherein a first dose of the blinatumomab or the blinatumomab variant is administered to the subject concomitant with the administration of a first dose of the pembrolizumab, the pembrolizumab variant or the antigen-binding fragment thereof.

79. The medicament of claim 77, wherein the blinatumomab or the blinatumomab variant is administered daily.

80. The medicament of claim 77, wherein a secondary dose of pembrolizumab, pembrolizumab variant or antigen-binding fragment thereof is administered approximately 21 days after the first dose of the pembrolizumab, pembrolizumab variant or antigen-binding fragment thereof.

81. The medicament of claim 80, wherein one or more additional secondary doses of pembrolizumab, pembrolizumab variant or antigen-binding fragment thereof are administered approximately every 21 days.

82. The medicament of claim 77, wherein the pembrolizumab, the pembrolizumab variant or the antigen-binding fragment thereof is administered at a dose of about 200 mg.

83. The medicament of claim 77, wherein the blinatumomab or the blinatumomab variant is administered at an initial dose of at least about 9 μg/d.

84. The medicament of claim 83, wherein the blinatumomab or the blinatumomab variant is administered at a maintenance dose of about 28 μg/d, about 56 μg/d or about 112/d μg.

85. The medicament of claim 79, wherein the blinatumomab or the blinatumomab variant is administered in a first treatment cycle, followed by a treatment-free cycle, followed by one or more consolidation cycles.

86. The medicament of claim 85, wherein the first treatment cycle is between about 49 and about 63 days.

87. The medicament of claim 86, wherein the first treatment cycle is about 56 days.

88. The medicament of claim 85, wherein the treatment-free cycle is between about 14 and about 28 days.

89. The medicament of claim 88, wherein the treatment-free cycle is about 21 days.

90. The medicament of claim 89, wherein each of the one or more consolidation cycles are between about 14 and about 28 days.

91. The medicament of claim 90, wherein each of the one or more consolidation cycles are about 21 days.

92. The medicament of claim 77, wherein the first dose of the blinatumomab or the blinatumomab variant is administered to the subject on day 1 and the first dose of the pembrolizumab, the pembrolizumab variant or the antigen-binding fragment thereof is administered to the subject on day 1.

93. The medicament of claim 77, wherein the first dose of the blinatumomab or the blinatumomab variant is administered to the subject on day 1 and the first dose of the pembrolizumab, the pembrolizumab variant or the antigen-binding fragment thereof is administered to the subject on about day 15.

94. The medicament of claim 77, wherein the first dose of the blinatumomab or the blinatumomab variant is administered to the subject on day 1 and the first dose of the pembrolizumab, the pembrolizumab variant or the antigen-binding fragment thereof is administered to the subject on about day 19.

95. The medicament of claim 76, wherein the blinatumomab or the blinatumomab variant is administered by CIVI.

96. The medicament of claim 76, wherein the pembrolizumab, the pembrolizumab variant or the antigen-binding fragment thereof is administered by IV infusion.


SEQUENCE LISTING (33 sequences)

SEQ ID NO: 1 - protein, 10 aa, Artificial Sequence
  source: /note=“Description of Artificial Sequence Synthetic peptide”
GYTFTRYTMH

SEQ ID NO: 2 - protein, 17 aa, Artificial Sequence
  source: /note=“Description of Artificial Sequence Synthetic peptide”
YINPSRGYTNYNQKFKD

SEQ ID NO: 3 - protein, 10 aa, Artificial Sequence
  source: /note=“Description of Artificial Sequence Synthetic peptide”
YYDDHYCLDY

SEQ ID NO: 4 - protein, 10 aa, Artificial Sequence
  source: /note=“Description of Artificial Sequence Synthetic peptide”
RASSSVSYMN

SEQ ID NO: 5 - protein, 7 aa, Artificial Sequence
  source: /note=“Description of Artificial Sequence Synthetic peptide”
DTSKVAS

SEQ ID NO: 6 - protein, 9 aa, Artificial Sequence
  source: /note=“Description of Artificial Sequence Synthetic peptide”
QQWSSNPLT

SEQ ID NO: 7 - protein, 10 aa, Artificial Sequence
  source: /note=“Description of Artificial Sequence Synthetic peptide”
GYAFSSYWMN

SEQ ID NO: 8 - protein, 17 aa, Artificial Sequence
  source: /note=“Description of Artificial Sequence Synthetic peptide”
QIWPGDGDTNYNGKFKG

SEQ ID NO: 9 - protein, 15 aa, Artificial Sequence
  source: /note=“Description of Artificial Sequence Synthetic peptide”
RETTTVGRYYYAMDY

SEQ ID NO: 10 - protein, 15 aa, Artificial Sequence
  source: /note=“Description of Artificial Sequence Synthetic peptide”
KASQSVDYDGDSYLN

SEQ ID NO: 11 - protein, 7 aa, Artificial Sequence
  source: /note=“Description of Artificial Sequence Synthetic peptide”
DASNLVS

SEQ ID NO: 12 - protein, 9 aa, Artificial Sequence
  source: /note=“Description of Artificial Sequence Synthetic peptide”
QQSTEDPWT

SEQ ID NO: 13 - protein, 124 aa, Artificial Sequence
  source: /note=“Description of Artificial Sequence Synthetic polypeptide”
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 14 - DNA, 372 bp, Artificial Sequence
  source: /note=“Description of Artificial Sequence Synthetic polynucleotide”
caggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggccaagggaccacggtcaccgtctcctcc

SEQ ID NO: 15 - protein, 111 aa, Artificial Sequence
  source: /note=“Description of Artificial Sequence Synthetic polypeptide”
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 16 - DNA, 333 bp, Artificial Sequence
  source: /note=“Description of Artificial Sequence Synthetic polynucleotide”
gatatccagctgacccagtctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctcgagatcaaa

SEQ ID NO: 17 - protein, 119 aa, Artificial Sequence
  source: /note=“Description of Artificial Sequence Synthetic polypeptide”
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 18 - DNA, 357 bp, Artificial Sequence
  source: /note=“Description of Artificial Sequence Synthetic polynucleotide”
gatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctca

SEQ ID NO: 19 - protein, 106 aa, Artificial Sequence
  source: /note=“Description of Artificial Sequence Synthetic polypeptide”
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 20 - DNA, 318 bp, Artificial Sequence
  source: /note=“Description of Artificial Sequence Synthetic polynucleotide”
gacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 21 - protein, 498 aa, Artificial Sequence
  source: /note=“Description of Artificial Sequence Synthetic polypeptide”
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 22 - DNA, 1494 bp, Artificial Sequence
  source: /note=“Description of Artificial Sequence Synthetic polynucleotide”
gatatccagctgacccagtctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctcgagatcaaaggtggtggtggttctggcggcggcggctccggtggtggtggttctcaggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggccaagggaccacggtcaccgtctcctccggaggtggtggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 23 - protein, 447 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 218 aa, Artificial Sequence
  source: /note=“Description of Artificial Sequence Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 120 aa, Artificial Sequence [sequence omitted]

SEQ ID NO: 26 - protein, 111 aa, Artificial Sequence [sequence omitted]

SEQ ID NO: 27 - protein, 5 aa, Artificial Sequence
  source: /note=“Description of Artificial Sequence Synthetic peptide”
NYYMY

SEQ ID NO: 28 - protein, 12 aa, Artificial Sequence
  source: /note=“Description of Artificial Sequence Synthetic peptide”
GINPSNGGTNFN

SEQ ID NO: 29 - protein, 11 aa, Artificial Sequence
  source: /note=“Description of Artificial Sequence Synthetic peptide”
RDYRFDMGFDY

SEQ ID NO: 30 - protein, 15 aa, Artificial Sequence
  source: /note=“Description of Artificial Sequence Synthetic peptide”
RASKGVSTSGYSYLH

SEQ ID NO: 31 - protein, 7 aa, Artificial Sequence
  source: /note=“Description of Artificial Sequence Synthetic peptide”
LASYLES

SEQ ID NO: 32 - protein, 9 aa, Artificial Sequence
  source: /note=“Description of Artificial Sequence Synthetic peptide”
QHSRDLPLT

SEQ ID NO: 33 - protein, 5 aa, Artificial Sequence
  source: /note=“Description of Artificial Sequence Synthetic peptide”
GGGGS